SEQUENCE LISTING (23 sequences)

SEQ ID NO: 1 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 2 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 3 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 4 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 5 - protein, 1252 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 6 - protein, 1278 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 7 - protein, 1297 aa, Clostridium botulinum
MPVNIKNFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 8 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 9 - DNA, 3891 bp, Clostridium botulinum
atgccatttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtaagagggataataacttctaaaactaaatcattagataaaggatacaataaggcattaaatgatttatgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcatcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa

SEQ ID NO: 10 - DNA, 3876 bp, Clostridium botulinum
atgccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgttaaagctccaggaatatgtattgatgttgataatgaagatttgttctttatagctgataaaaatagtttttcagatgatttatctaaaaacgaaagaatagaatataatacacagagtaattatatagaaaatgacttccctataaatgaattaattttagatactgatttaataagtaaaatagaattaccaagtgaaaatacagaatcacttactgattttaatgtagatgttccagtatatgaaaaacaacccgctataaaaaaaatttttacagatgaaaataccatctttcaatatttatactctcagacatttcctctagatataagagatataagtttaacatcttcatttgatgatgcattattattttctaacaaagtttattcatttttttctatggattatattaaaactgctaataaagtggtagaagcaggattatttgcaggttgggtgaaacagatagtaaatgattttgtaatcgaagctaataaaagcaatactatggataaaattgcagatatatctctaattgttccttatataggattagctttaaatgtaggaaatgaaacagctaaaggaaattttgaaaatgcttttgagattgcaggagccagtattctactagaatttataccagaacttttaatacctgtagttggagcctttttattagaatcatatattgacaataaaaataaaattattaaaacaatagataatgctttaactaaaagaaatgaaaaatggagtgatatgtacggattaatagtagcgcaatggctctcaacagttaatactcaattttatacaataaaagagggaatgtataaggctttaaattatcaagcacaagcattggaagaaataataaaatacagatataatatatattctgaaaaagaaaagtcaaatattaacatcgattttaatgatataaattctaaacttaatgagggtattaaccaagctatagataatataaataattttataaatggatgttctgtatcatatttaatgaaaaaaatgattccattagctgtagaaaaattactagactttgataatactctcaaaaaaaatttgttaaattatatagatgaaaataaattatatttgattggaagtgcagaatatgaaaaatcaaaagtaaataaatacttgaaaaccattatgccgtttgatctttcaatatataccaatgatacaatactaatagaaatgtttaataaatataatagcgaaattttaaataatattatcttaaatttaagatataaggataataatttaatagatttatcaggatatggggcaaaggtagaggtatatgatggagtcgagcttaatgataaaaatcaatttaaattaactagttcagcaaatagtaagattagagtgactcaaaatcagaatatcatatttaatagtgtgttccttgattttagcgttagcttttggataagaatacctaaatataagaatgatggtatacaaaattatattcataatgaatatacaataattaattgtatgaaaaataattcgggctggaaaatatctattaggggtaataggataatatggactttaattgatataaatggaaaaaccaaatcggtattttttgaatataacataagagaagatatatcagagtatataaatagatggttttttgtaactattactaataatttgaataacgctaaaatttatattaatggtaagctagaatcaaatacagatattaaagatataagagaagttattgctaatggtgaaataatatttaaattagatggtgatatagatagaacacaatttatttggatgaaatatttcagtatttttaatacggaattaagtcaatcaaatattgaagaaagatataaaattcaatcatatagcgaatatttaaaagatttttggggaaatcctttaatgtacaataaagaatattatatgtttaatgcggggaataaaaattcatatattaaactaaagaaagattcacctgtaggtgaaattttaacacgtagcaaatataatcaaaattctaaatatataaattatagagatttatatattggagaaaaatttattataagaagaaagtcaaattctcaatctataaatgatgatatagttagaaaagaagattatatatatctagatttttttaatttaaatcaagagtggagagtatatacctataaatattttaagaaagaggaagaaaaattgtttttagctcctataagtgattctgatgagttttacaatactatacaaataaaagaatatgatgaacagccaacatatagttgtcagttgctttttaaaaaagatgaagaaagtactgatgagataggattgattggtattcatcgtttctacgaatctggaattgtatttgaagagtataaagattatttttgtataagtaaatggtacttaaaagaggtaaaaaggaaaccatataatttaaaattgggatgtaattggcagtttattcctaaagatgaagggtggactgaataa

SEQ ID NO: 11 - DNA, 3876 bp, Clostridium botulinum
atgccaataacaattaacaactttaattattcagatcctgttgataataaaaatattttatatttagatactcatttaaatacactagctaatgagcctgaaaaagcctttcgcattacaggaaatatatgggtaatacctgatagattttcaagaaattctaatccaaatttaaataaacctcctcgagttacaagccctaaaagtggttattatgatcctaattatttgagtactgattctgacaaagatacatttttaaaagaaattataaagttatttaaaagaattaattctagagaaataggagaagaattaatatatagactttcgacagatataccctttcctgggaataacaatactccaattaatacttttgattttgatgtagattttaacagtgttgatgttaaaactagacaaggtaacaactgggttaaaactggtagcataaatcctagtgttataataactggacctagagaaaacattatagatccagaaacttctacgtttaaattaactaacaatacttttgcggcacaagaaggatttggtgctttatcaataatttcaatatcacctagatttatgctaacatatagtaatgcaactaatgatgtaggagagggtagattttctaagtctgaattttgcatggatccaatactaattttaatgcatgaacttaatcatgcaatgcataatttatatggaatagctataccaaatgatcaaacaatttcatctgtaactagtaatattttttattctcaatataatgtgaaattagagtatgcagaaatatatgcatttggaggtccaactatagaccttattcctaaaagtgcaaggaaatattttgaggaaaaggcattggattattatagatctatagctaaaagacttaatagtataactactgcaaatccttcaagctttaataaatatataggggaatataaacagaaacttattagaaagtatagattcgtagtagaatcttcaggtgaagttacagtaaatcgtaataagtttgttgagttatataatgaacttacacaaatatttacagaatttaactacgctaaaatatataatgtacaaaataggaaaatatatctttcaaatgtatatactccggttacggcgaatatattagacgataatgtttatgatatacaaaatggatttaatatacctaaaagtaatttaaatgtactatttatgggtcaaaatttatctcgaaatccagcattaagaaaagtcaatcctgaaaatatgctttatttatttacaaaattttgtcataaagcaatagatggtagatcattatataataaaacattagattgtagagagcttttagttaaaaatactgacttaccctttataggtgatattagtgatgttaaaactgatatatttttaagaaaagatattaatgaagaaactgaagttatatactatccggacaatgtttcagtagatcaagttattctcagtaagaatacctcagaacatggacaactagatttattataccctagtattgacagtgagagtgaaatattaccaggggagaatcaagtcttttatgataatagaactcaaaatgttgattatttgaattcttattattacctagaatctcaaaaactaagtgataatgttgaagattttacttttacgagatcaattgaggaggctttggataatagtgcaaaagtatatacttactttcctacactagctaataaagtaaatgcgggtgttcaaggtggtttatttttaatgtgggcaaatgatgtagttgaagattttactacaaatattctaagaaaagatacattagataaaatatcagatgtatcagctattattccctatataggacccgcattaaatataagtaattctgtaagaagaggaaattttactgaagcatttgcagttactggtgtaactattttattagaagcatttcctgaatttacaatacctgcacttggtgcatttgtgatttatagtaaggttcaagaaagaaacgagattattaaaactatagataattgtttagaacaaaggattaagagatggaaagattcatatgaatggatgatgggaacgtggttatccaggattattactcaatttaataatataagttatcaaatgtatgattctttaaattatcaggcaggtgcaatcaaagctaaaatagatttagaatataaaaaatattcaggaagtgataaagaaaatataaaaagtcaagttgaaaatttaaaaaatagtttagatgtaaaaatttcggaagcaatgaataatataaataaatttatacgagaatgttccgtaacatatttatttaaaaatatgttacctaaagtaattgatgaattaaatgagtttgatcgaaatactaaagcaaaattaattaatcttatagatagtcataatattattctagttggtgaagtagataaattaaaagcaaaagtaaataatagctttcaaaatacaataccctttaatattttttcatatactaataattctttattaaaagatataattaatgaatatttcaataatattaatgattcaaaaattttgagcctacaaaacagaaaaaatactttagtggatacatcaggatataatgcagaagtgagtgaagaaggcgatgttcagcttaatccaatatttccatttgactttaaattaggtagttcaggggaggatagaggtaaagttatagtaacccagaatgaaaatattgtatataattctatgtatgaaagttttagcattagtttttggattagaataaataaatgggtaagtaatttacctggatatactataattgatagtgttaaaaataactcaggttggagtataggtattattagtaattttttagtatttactttaaaacaaaatgaagatagtgaacaaagtataaattttagttatgatatatcaaataatgctcctggatacaataaatggttttttgtaactgttactaacaatatgatgggaaatatgaagatttatataaatggaaaattaatagatactataaaagttaaagaactaactggaattaattttagcaaaactataacatttgaaataaataaaattccagataccggtttgattacttcagattctgataacatcaatatgtggataagagatttttatatatttgctaaagaattagatggtaaagatattaatatattatttaatagcttgcaatatactaatgttgtaaaagattattggggaaatgatttaagatataataaagaatattatatggttaatatagattatttaaatagatatatgtatgcgaactcacgacaaattgtttttaatacacgtagaaataataatgacttcaatgaaggatataaaattataataaaaagaatcagaggaaatacaaatgatactagagtacgaggaggagatattttatattttgatatgacaattaataacaaagcatataatttgtttatgaagaatgaaactatgtatgcagataatcatagtactgaagatatatatgctataggtttaagagaacaaacaaaggatataaatgataatattatatttcaaatacaaccaatgaataatacttattattacgcatctcaaatatttaaatcaaattttaatggagaaaatatttctggaatatgttcaataggtacttatcgttttagacttggaggtgattggtatagacacaattatttggtgcctactgtgaagcaaggaaattatgcttcattattagaatcaacatcaactcattggggttttgtacctgtaagtgaataa

SEQ ID NO: 12 - DNA, 3831 bp, Clostridium botulinum
atgacatggccagtaaaagattttaattatagtgatcctgttaatgacaatgatatattatatttaagaataccacaaaataagttaattactacacctgtaaaagcttttatgattactcaaaatatttgggtaataccagaaagattttcatcagatactaatccaagtttaagtaaaccgcccagacctacttcaaagtatcaaagttattatgatcctagttatttatctactgatgaacaaaaagatacatttttaaaagggattataaaattatttaaaagaattaatgaaagagatataggaaaaaaattaataaattatttagtagttggttcaccttttatgggagattcaagtacgcctgaagatacatttgattttacacgtcatactactaatattgcagttgaaaagtttgaaaatggtagttggaaagtaacaaatattataacaccaagtgtattgatatttggaccacttcctaatatattagactatacagcatcccttacattgcaaggacaacaatcaaatccatcatttgaagggtttggaacattatctatactaaaagtagcacctgaatttttgttaacatttagtgatgtaacatctaatcaaagttcagctgtattaggcaaatctatattttgtatggatccagtaatagctttaatgcatgagttaacacattctttgcatcaattatatggaataaatataccatctgataaaaggattcgtccacaagttagcgagggatttttctctcaagatggacccaacgtacaatttgaggaattatatacatttggaggattagatgttgaaataatacctcaaattgaaagatcacaattaagagaaaaagcattaggtcactataaagatatagcgaaaagacttaataatattaataaaactattccttctagttggattagtaatatagataaatataaaaaaatattttctgaaaagtataattttgataaagataatacaggaaattttgttgtaaatattgataaattcaatagcttatattcagacttgactaatgttatgtcagaagttgtttattcttcgcaatataatgttaaaaacaggactcattatttttcaaggcattatctacctgtatttgcaaatatattagatgataatatttatactataagagatggttttaatttaacaaataaaggttttaatatagaaaattcgggtcagaatatagaaaggaatcctgcactacaaaagcttagttcagaaagtgtagtagatttatttacaaaagtatgtttaagattaacaaaaaatagtagagatgattcaacatgtattaaagttaaaaataatagattaccttatgtagctgataaagatagcatttcacaagaaatatttgaaaataaaattattacagatgagactaatgtacaaaattattcagataaattttcattagatgaatctattttagatgggcaagttcctattaatcctgaaatagtagatccactattacccaatgttaatatggaacctttaaatcttccaggtgaagaaatagtattttatgatgatattactaaatatgttgattatttaaattcttattattatttggaatctcaaaaattaagtaataatgttgaaaatattactcttacaacttcagttgaagaagcattaggttatagcaataagatatacacatttttacctagcttagctgaaaaagtgaataaaggtgttcaagcaggtttattcttaaattgggcgaatgaagtagttgaggattttactacaaatattatgaagaaagatacattggataaaatatcagatgtatcagtaataattccatatataggacctgccttaaatataggaaattcagcattaaggggaaattttaatcaagcatttgcaacagctggtgtagcttttttattagagggatttccagagtttactatacctgcactcggtgtatttaccttttatagttctattcaagaaagagagaaaattattaaaactatagaaaattgtttggaacaaagagttaagagatggaaagattcatatcaatggatggtatcaaattggttgtcaagaattactactcaatttaatcatataaattatcaaatgtatgattctttaagttatcaggcagatgcaatcaaagctaaaatagatttagaatataaaaaatactcaggaagtgataaagaaaatataaaaagtcaagttgaaaatttaaaaaatagtttagatgtaaaaatttcggaagcaatgaataatataaataaatttatacgagaatgttctgtaacatacttatttaaaaatatgctccctaaagtaattgacgaattaaataagtttgatttaagaactaaaacagaattaattaatcttatagatagtcataatattattctagttggtgaagtagatagattaaaagcaaaagtaaatgagagttttgaaaatacaatgccttttaatattttttcatatactaataattctttattaaaagatataattaatgaatatttcaatagtattaatgattcaaaaattttgagcttacaaaacaaaaaaaatgctttagtggatacatcaggatataatgcagaagtgagggtaggagataatgttcaacttaatacgatatatacaaatgactttaaattaagtagttcaggagataaaattatagtaaatttaaataataatattttatatagcgctatttatgagaactctagtgttagtttttggattaagatatctaaagatttaactaattctcataatgaatatacaataattaacagtatagaacaaaattctgggtggaaattatgtattaggaatggcaatatagaatggattttacaagatgttaatagaaagtataaaagtttaatttttgattatagtgaatcattaagtcatacaggatatacaaataaatggttttttgttactataactaataatataatggggtatatgaaactttatataaatggagaattaaagcagagtcaaaaaattgaagatttagatgaggttaagttagataaaaccatagtatttggaatagatgagaatatagatgagaatcagatgctttggattagagattttaatattttttctaaagaattaagtaatgaagatattaatattgtatatgagggacaaatattaagaaatgttattaaagattattggggaaatcctttgaagtttgatacagaatattatattattaatgataattatatagataggtatatagcacctgaaagtaatgtacttgtacttgttcagtatccagatagatctaaattatatactggaaatcctattactattaaatcagtatctgataagaatccttatagtagaattttaaatggagataatataattcttcatatgttatataatagtaggaaatatatgataataagagatactgatacaatatatgcaacacaaggaggagagtgttcacaaaattgtgtatatgcattaaaattacagagtaatttaggtaattatggtataggtatatttagtataaaaaatattgtatctaaaaataaatattgtagtcaaattttctctagttttagggaaaatacaatgcttctagcagatatatataaaccttggagattttcttttaaaaatgcatacacgccagttgcagtaactaattatgaaacaaaactattatcaacttcatctttttggaaatttatttctagggatccaggatgggtagagtaa

SEQ ID NO: 13 - DNA, 3756 bp, Clostridium botulinum
atgccaaaaattaatagttttaattataatgatcctgttaatgatagaacaattttatatattaaaccaggcggttgtcaagaattttataaatcatttaatattatgaaaaatatttggataattccagagagaaatgtaattggtacaaccccccaagattttcatccgcctacttcattaaaaaatggagatagtagttattatgaccctaattatttacaaagtgatgaagaaaaggatagatttttaaaaatagtcacaaaaatatttaatagaataaataataatctttcaggagggattttattagaagaactgtcaaaagctaatccatatttagggaatgataatactccagataatcaattccatattggtgatgcatcagcagttgagattaaattctcaaatggtagccaagacatactattacctaatgttattataatgggagcagagcctgatttatttgaaactaacagttccaatatttctctaagaaataattatatgccaagcaatcaccgttttggatcaatagctatagtaacattctcacctgaatattcttttagatttaatgataattgtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttacgaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataaggaaatcaatatgtatcgaaataaataatggtgagttattttttgtggcttccgagaatagttataatgatgataatataaatactcctaaagaaattgacgatacagtaacttcaaataataattatgaaaatgatttagatcaggttattttaaattttaatagtgaatcagcacctggactttcagatgaaaaattaaatttaactatccaaaatgatgcttatataccaaaatatgattctaatggaacaagtgatatagaacaacatgatgttaatgaacttaatgtatttttctatttagatgcacagaaagtgcccgaaggtgaaaataatgtcaatctcacctcttcaattgatacagcattattagaacaacctaaaatatatacatttttttcatcagaatttattaataatgtcaataaacctgtgcaagcagcattatttgtaagctggatacaacaagtgttagtagattttactactgaagctaaccaaaaaagtactgttgataaaattgcagatatttctatagttgttccatatataggtcttgctttaaatataggaaatgaagcacaaaaaggaaattttaaagatgcacttgaattattaggagcaggtattttattagaatttgaacccgagcttttaattcctacaattttagtattcacgataaaatcttttttaggttcatctgataataaaaataaagttattaaagcaataaataatgcattgaaagaaagagatgaaaaatggaaagaagtatatagttttatagtatcgaattggatgactaaaattaatacacaatttaataaaagaaaagaacaaatgtatcaagctttacaaaatcaagtaaatgcaattaaaacaataatagaatctaagtataatagttatactttagaggaaaaaaatgagcttacaaataaatatgatattaagcaaatagaaaatgaacttaatcaaaaggtttctatagcaatgaataatatagacaggttcttaactgaaagttctatatcctatttaatgaaaataataaatgaagtaaaaattaataaattaagagaatatgatgagaatgtcaaaacgtatttattgaattatattatacaacatggatcaatcttgggagagagtcagcaagaactaaattctatggtaactgataccctaaataatagtattccttttaagctttcttcttatacagatgataaaattttaatttcatattttaataaattctttaagagaattaaaagtagttcagttttaaatatgagatataaaaatgataaatacgtagatacttcaggatatgattcaaatataaatattaatggagatgtatataaatatccaactaataaaaatcaatttggaatatataatgataaacttagtgaagttaatatatctcaaaatgattacattatatatgataataaatataaaaattttagtattagtttttgggtaagaattcctaactatgataataagatagtaaatgttaataatgaatacactataataaattgtatgagagataataattcaggatggaaagtatctcttaatcataatgaaataatttggacattcgaagataatcgaggaattaatcaaaaattagcatttaactatggtaacgcaaatggtatttctgattatataaataagtggatttttgtaactataactaatgatagattaggagattctaaactttatattaatggaaatttaatagatcaaaaatcaattttaaatttaggtaatattcatgttagtgacaatatattatttaaaatagttaattgtagttatacaagatatattggtattagatattttaatatttttgataaagaattagatgaaacagaaattcaaactttatatagcaatgaacctaatacaaatattttgaaggatttttggggaaattatttgctttatgacaaagaatactatttattaaatgtgttaaaaccaaataactttattgataggagaaaagattctactttaagcattaataatataagaagcactattcttttagctaatagattatatagtggaataaaagttaaaatacaaagagttaataatagtagtactaacgataatcttgttagaaagaatgatcaggtatatattaattttgtagccagcaaaactcacttatttccattatatgctgatacagctaccacaaataaagagaaaacaataaaaatatcatcatctggcaatagatttaatcaagtagtagttatgaattcagtaggaaattgtacaatgaattttaaaaataataatggaaataatattgggttgttaggtttcaaggcagatactgtcgttgctagtacttggtattatacacatatgagagatcatacaaacagcaatggatgtttttggaactttatttctgaagaacatggatggcaagaaaaataa

SEQ ID NO: 14 - DNA, 3837 bp, Clostridium botulinum
atgccagttgtaataaatagttttaattataatgaccctgttaatgatgatacaattttatacatgcagataccatatgaagaaaaaagtaaaaaatattataaagcttttgagattatgcgtaatgtttggataattcctgagagaaatacaataggaacggatcctagtgattttgatccaccggcttcattagagaacggaagcagtgcttattatgatcctaattatttaaccactgatgctgaaaaagatagatatttaaaaacaacgataaaattatttaagagaattaatagtaatcctgcaggggaagttttgttacaagaaatatcatatgctaaaccatatttaggaaatgaacacacgccaattaatgaattccatccagttactagaactacaagtgttaatataaaatcatcaactaatgttaaaagttcaataatattgaatcttcttgtattgggagcaggacctgatatatttgaaaattcttcttaccccgttagaaaactaatggattcaggtggagtttatgacccaagtaatgatggttttggatcaattaatatcgtgacattttcacctgaatatgaatatacttttaatgatattagtggagggtataacagtagtacagaatcatttattgcagatcctgcaatttcactagctcatgaattgatacatgcactgcatggattatacggggctaggggagttacttataaagagactataaaagtaaagcaagcacctcttatgatagccgaaaaacccataaggctagaagaatttttaacctttggaggtcaggatttaaatattattactagtgctatgaaggaaaaaatatataacaatcttttagctaactatgaaaaaatagctactagacttagtagagttaatagtgctcctcctgaatatgatattaatgaatataaagattattttcaatggaagtatgggctagataaaaatgctgatggaagttatactgtaaatgaaaataaatttaatgaaatttataaaaaattatatagctttacagagattgacttagcaaataaatttaaagtaaaatgtagaaatacttattttattaaatatggatttttaaaagttccaaatttgttagatgatgatatttatactgtatcagaggggtttaatataggtaatttagcagtaaacaatcgcggacaaaatataaagttaaatcctaaaattattgattccattccagataaaggtctagtggaaaagatcgttaaattttgtaagagcgttattcctagaaaaggtacaaaggcgccaccgcgactatgcattagagtaaataatagggagttattttttgtagcttcagaaagtagctataatgaaaatgatattaatacacctaaagaaattgacgatacaacaaatctaaataataattatagaaataatttagatgaagttattttagattataatagtgagacaatacctcaaatatcaaatcaaacattaaatacacttgtacaagacgatagttatgtgccaagatatgattctaatggaacaagtgaaatagaggaacataatgttgttgaccttaatgtatttttctatttacatgcacaaaaagtaccagaaggtgaaactaatataagtttaacttcttcaattgatacggcattatcagaagaatcgcaagtatatacattcttttcttcagagtttattaatactatcaataaacctgtacacgcagcactatttataagttggataaatcaagtaataagagattttactactgaagctacacaaaaaagtacttttgataagattgcagacatatctttagttgtaccatatgtaggtcttgctttaaatataggtaatgaggtacaaaaagaaaattttaaggaggcatttgaattattaggagcgggtattttattagaatttgtgccagagcttttaattcctacaattttagtgtttacaataaaatcctttataggttcatctgagaataaaaataaaatcattaaagcaataaataattcattaatggaaagagaaacaaagtggaaagaaatatatagttggatagtatcaaattggcttactagaattaatacacaatttaataaaagaaaagaacaaatgtatcaagctttgcaaaatcaagtagatgcaataaaaacagtaatagaatataaatataataattatacttcagatgagagaaatagacttgaatctgaatataatatcaataatataagagaagaattgaacaaaaaagtttctttagcaatggaaaatatagagagatttataacagagagttctatattttatttaatgaagttaataaatgaagccaaagttagtaaattaagagaatatgatgaaggcgttaaggaatatttgctagactatatttcagaacatagatcaattttaggaaatagtgtacaagaattaaatgatttagtgactagtactctgaataatagtattccatttgaactttcttcatatactaatgataaaattctaattttatattttaataaattatataaaaaaattaaagataactctattttagatatgcgatatgaaaataataaatttatagatatctctggatatggttcaaatataagcattaatggagatgtatatatttattcaacaaatagaaatcaatttggaatatatagtagtaagcctagtgaagttaatatagctcaaaataatgatattatatacaatggtagatatcaaaattttagtattagtttctgggtaaggattcctaaatacttcaataaagtgaatcttaataatgaatatactataatagattgtataaggaataataattcaggatggaaaatatcacttaattataataaaataatttggactttacaagatactgctggaaataatcaaaaactagtttttaattatacacaaatgattagtatatctgattatataaataaatggatttttgtaactattactaataatagattaggcaattctagaatttacatcaatggaaatttaatagatgaaaaatcaatttcgaatttaggtgatattcatgttagtgataatatattatttaaaattgttggttgtaatgatacaagatatgttggtataagatattttaaagtttttgatacggaattaggtaaaacagaaattgagactttatatagtgatgagccagatccaagtatcttaaaagacttttggggaaattatttgttatataataaaagatattatttattgaatttactaagaacagataagtctattactcagaattcaaactttctaaatattaatcaacaaagaggtgtttatcagaaaccaaatattttttccaacactagattatatacaggagtagaagttattataagaaaaaatggatctacagatatatctaatacagataattttgttagaaaaaatgatctggcatatattaatgtagtagatcgtgatgtagaatatcggctatatgctgatatatcaattgcaaaaccagagaaaataataaaattaataagaacatctaattcaaacaatagcttaggtcaaattatagttatggattcaataggaaataattgcacaatgaattttcaaaacaataatgggggcaatataggattactaggttttcattcaaataatttggttgctagtagttggtattataacaatatacgaaaaaatactagcagtaatggatgcttttggagttttatttctaaagagcatggatggcaagaaaactaa

SEQ ID NO: 15 - DNA, 3894 bp, Clostridium botulinum
  misc_feature (20)..(20): n is a, c, g, or t
atgccagttaatataaaaanctttaattataatgaccctattaataatgatgacattattatgatggaaccattcaatgacccagggccaggaacatattataaagcttttaggattatagatcgtatttggatagtaccagaaaggtttacttatggatttcaacctgaccaatttaatgccagtacaggagtttttagtaaagatgtctacgaatattacgatccaacttatttaaaaaccgatgctgaaaaagataaatttttaaaaacaatgattaaattatttaatagaattaattcaaaaccatcaggacagagattactggatatgatagtagatgctataccttatcttggaaatgcatctacaccgcccgacaaatttgcagcaaatgttgcaaatgtatctattaataaaaaaattatccaacctggagctgaagatcaaataaaaggtttaatgacaaatttaataatatttggaccaggaccagttctaagtgataattttactgatagtatgattatgaatggccattccccaatatcagaaggatttggtgcaagaatgatgataagattttgtcctagttgtttaaatgtatttaataatgttcaggaaaataaagatacatctatatttagtagacgcgcgtattttgcagatccagctctaacgttaatgcatgaacttatacatgtgttacatggattatatggaattaagataagtaatttaccaattactccaaatacaaaagaatttttcatgcaacatagcgatcctgtacaagcagaagaactatatacattcggaggacatgatcctagtgttataagtccttctacggatatgaatatttataataaagcgttacaaaattttcaagatatagctaataggcttaatattgtttcaagtgcccaagggagtggaattgatatttccttatataaacaaatatataaaaataaatatgattttgttgaagatcctaatggaaaatatagtgtagataaggataagtttgataaattatataaggccttaatgtttggctttactgaaactaatctagctggtgaatatggaataaaaactaggtattcttattttagtgaatatttgccaccgataaaaactgaaaaattgttagacaatacaatttatactcaaaatgaaggctttaacatagctagtaaaaatctcaaaacggaatttaatggtcagaataaggcggtaaataaagaggcttatgaagaaatcagcctagaacatctcgttatatatagaatagcaatgtgcaagcctgtaatgtacaaaaataccggtaaatctgaacagtgtattattgttaataatgaggatttatttttcatagctaataaagatagtttttcaaaagatttagctaaagcagaaactatagcatataatacacaaaataatactatagaaaataatttttctatagatcagttgattttagataatgatttaagcagtggcatagacttaccaaatgaaaacacagaaccatttacaaattttgacgacatagatatccctgtgtatattaaacaatctgctttaaaaaaaatttttgtggatggagatagcctttttgaatatttacatgctcaaacatttccttctaatatagaaaatctacaactaacgaattcattaaatgatgctttaagaaataataataaagtctatacttttttttctacaaaccttgttgaaaaagctaatacagttgtaggtgcttcactttttgtaaactgggtaaaaggagtaatagatgattttacatctgaatccacacaaaaaagtactatagataaagtttcagatgtatccataattattccctatataggacctgctttgaatgtaggaaatgaaacagctaaagaaaattttaaaaatgcttttgaaataggtggagccgctatcttaatggagtttattccagaacttattgtacctatagttggattttttacattagaatcatatgtaggaaataaagggcatattattatgacgatatccaatgctttaaagaaaagggatcaaaaatggacagatatgtatggtttgatagtatcgcagtggctctcaacggttaatactcaattttatacaataaaagaaagaatgtacaatgctttaaataatcaatcacaagcaatagaaaaaataatagaagatcaatataatagatatagtgaagaagataaaatgaatattaacattgattttaatgatatagattttaaacttaatcaaagtataaatttagcaataaacaatatagatgattttataaaccaatgttctatatcatatctaatgaatagaatgattccattagctgtaaaaaagttaaaagactttgatgataatcttaagagagatttattggagtatatagatacaaatgaactatatttacttgatgaagtaaatattctaaaatcaaaagtaaatagacacctaaaagacagtataccatttgatctttcactatataccaaggacacaattttaatacaagtttttaataattatattagtaatattagtagtaatgctattttaagtttaagttatagaggtgggcgtttaatagattcatctggatatggtgcaactatgaatgtaggttcagatgttatctttaatgatataggaaatggtcaatttaaattaaataattctgaaaatagtaatattacggcacatcaaagtaaattcgttgtatatgatagtatgtttgataattttagcattaacttttgggtaaggactcctaaatataataataatgatatacaaacttatcttcaaaatgagtatacaataattagttgtataaaaaatgactcaggatggaaagtatctattaagggaaatagaataatatggacattaatagatgttaatgcaaaatctaaatcaatatttttcgaatatagtataaaagataatatatcagattatataaataaatggttttccataactattactaatgatagattaggtaacgcaaatatttatataaatggaagtttgaaaaaaagtgaaaaaattttaaacttagatagaattaattctagtaatgatatagacttcaaattaattaattgtacagatactactaaatttgtttggattaaggattttaatatttttggtagagaattaaatgctacagaagtatcttcactatattggattcaatcatctacaaatactttaaaagatttttgggggaatcctttaagatacgatacacaatactatctgtttaatcaaggtatgcaaaatatctatataaagtattttagtaaagcttctatgggggaaactgcaccacgtacaaactttaataatgcagcaataaattatcaaaatttatatcttggtttacgatttattataaaaaaagcatcaaattctcggaatataaataatgataatatagtcagagaaggagattatatatatcttaatattgataatatttctgatgaatcttacagagtatatgttttggtgaattctaaagaaattcaaactcaattatttttagcacccataaatgatgatcctacgttctatgatgtactacaaataaaaaaatattatgaaaaaacaacatataattgtcagatactttgcgaaaaagatactaaaacatttgggctgtttggaattggtaaatttgttaaagattatggatatgtttgggatacctatgataattatttttgcataagtcagtggtatctcagaagaatatctgaaaatataaataaattaaggttgggatgtaattggcaattcattcccgtggatgaaggatggacagaataa

SEQ ID NO: 16 - DNA, 3948 bp, Clostridium tetani
atgccaataaccataaataattttagatatagtgatcctgttaataatgatacaattattatgatggagccaccatactgtaagggtctagatatctattataaggctttcaaaataacagatcgtatttggatagtgccggaaaggtatgaatttgggacaaaacctgaagattttaacccaccatcttcattaatagaaggtgcatctgagtattacgatccaaattatttaaggactgattctgataaagatagatttttacaaaccatggtaaaactgtttaacagaattaaaaacaatgtagcaggtgaagccttattagataagataataaatgccataccttaccttggaaattcatattccttactagacaagtttgatacaaactctaattcagtatcttttaatttattagaacaagaccccagtggagcaactacaaaatcagcaatgctgacaaatttaataatatttggacctgggcctgttttaaataaaaatgaggttagaggtattgtattgagggtagataataaaaattacttcccatgtagagatggttttggctcaataatgcaaatggcattttgcccagaatatgtacctacctttgataatgtaatagaaaatattacgtcactcactattggcaaaagcaaatattttcaagatccagcattactattaatgcacgaacttatacatgtactacatggtttatacggaatgcaggtatcaagccatgaaattattccatccaaacaagaaatttatatgcagcatacatatccaataagtgctgaagaactattcacttttggcggacaggatgctaatcttataagtattgatataaaaaacgatttatatgaaaaaactttaaatgattataaagctatagctaacaaacttagtcaagtcactagctgcaatgatcccaacattgatattgatagctacaaacaaatatatcaacaaaaatatcaattcgataaagatagcaatggacaatatattgtaaatgaggataaatttcagatactatataatagcataatgtatggttttacagagattgaattgggaaaaaaatttaatataaaaactagactttcttattttagtatgaatcatgaccctgtaaaaattccaaatttattagatgatacaatttacaatgatacagaaggatttaatatagaaagcaaagatctgaaatctgaatataaaggacaaaatatgagggtaaatacaaatgcttttagaaatgttgatggatcaggcctagtttcaaaacttattggcttatgtaaaaaaattataccaccaacaaatataagagaaaatttatataatagaactgcatcattaacagatttaggaggagaattatgtataaaaattaaaaatgaagatttaacttttatagctgaaaaaaatagcttttcagaagaaccatttcaagatgaaatagttagttataatacaaaaaataaaccattaaattttaattattcgctagataaaattattgtagattataatctacaaagtaaaattacattacctaatgataggacaaccccagttacaaaaggaattccatatgctccagaatataaaagtaatgctgcaagtacaatagaaatacataatattgatgacaatacaatatatcaatatttgtatgctcaaaaatctcctacaactctacaaagaataactatgactaattctgttgatgacgcattaataaattccaccaaaatatattcatattttccatctgtaatcagtaaagttaaccaaggtgcacaaggaattttattcttacagtgggtgagagatataattgatgattttaccaatgaatcttcacaaaaaactactattgataaaatttcagatgtatccactattgttccttatataggacccgcattaaacattgtaaaacaaggctatgagggaaactttataggcgctttagaaactaccggagtggttttattattagaatatattccagaaattactttaccagtaattgcagctttatctatagcagaaagtagcacacaaaaagaaaagataataaaaacaatagataactttttagaaaaaagatatgaaaaatggattgaagtatataaactagtaaaagcaaaatggttaggcacagttaatacgcaattccaaaaaagaagttatcaaatgtatagatctttagaatatcaagtagatgcaataaaaaaaataatagactatgaatataaaatatattcaggacctgataaggaacaaattgccgacgaaattaataatctgaaaaacaaacttgaagaaaaggctaataaagcaatgataaacataaatatatttatgagggaaagttctagatcatttttagttaatcaaatgattaacgaagctaaaaagcagttattagagtttgatactcaaagcaaaaatattttaatgcagtatataaaagcaaattctaaatttataggtataactgaactaaaaaaattagaatcaaaaataaacaaagttttttcaacaccaattccattttcttattctaaaaatctggattgttgggttgataatgaagaagatatagatgttatattaaaaaagagtacaattttaaatttagatattaataatgatattatatcagatatatctgggtttaattcatctgtaataacatatccagatgctcaattggtgcccggaataaatggcaaagcaatacatttagtaaacaatgaatcttctgaagttatagtgcataaagctatggatattgaatataatgatatgtttaataattttaccgttagcttttggttgagggttcctaaagtatctgctagtcatttagaacaatatggcacaaatgagtattcaataattagctctatgaaaaaacatagtctatcaataggatctggttggagtgtatcacttaaaggtaataacttaatatggactttaaaagattccgcgggagaagttagacaaataacttttagggatttacctgataaatttaatgcttatttagcaaataaatgggtttttataactattactaatgatagattatcttctgctaatttgtatataaatggagtacttatgggaagtgcagaaattactggtttaggagctattagagaggataataatataacattaaaactagatagatgtaataataataatcaatacgtttctattgataaatttaggatattttgcaaagcattaaatccaaaagagattgaaaaattatacacaagttatttatctataacctttttaagagacttctggggaaaccctttacgatatgatacagaatattatttaataccagtagcttctagttctaaagatgttcaattgaaaaatataacagattatatgtatttgacaaatgcgccatcgtatactaacggaaaattgaatatatattatagaaggttatataatggactaaaatttattataaaaagatatacacctaataatgaaatagattcttttgttaaatcaggtgattttattaaattatatgtatcatataacaataatgagcacattgtaggttatccgaaagatggaaatgcctttaataatcttgatagaattctaagagtaggttataatgccccaggtatccctctttataaaaaaatggaagcagtaaaattgcgtgatttaaaaacctattctgtacaacttaaattatatgatgataaaaatgcatctttaggactagtaggtacccataatggtcaaataggcaacgatccaaatagggatatattaattgcaagcaactggtactttaatcatttaaaagataaaattttaggatgtgattggtactttgtacctacagatgaaggatggacaaatgattaa

SEQ ID NO: 17 - DNA, 1314 bp, Artificial Sequence
  BoNT/A1(0) LC
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatcaactgatctacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaa

SEQ ID NO: 18 - DNA, 30 bp, Artificial Sequence
  BoNT/A1(0) Activation loop
accaaaagcctggacaaaggctacaacaag

SEQ ID NO: 19 - DNA, 2547 bp, Artificial Sequence
  BoNT/A1(0) HC
gcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctgtaa

SEQ ID NO: 20 - DNA, 35 bp, Artificial Sequence
  primer
atacaccatggtatgccattcgtcaacaagcaatt

SEQ ID NO: 21 - DNA, 40 bp, Artificial Sequence
  primer
gcttttggatccggtttatttgctggtgatgataccgcgc

SEQ ID NO: 22 - DNA, 35 bp, Artificial Sequence
  primer
acaagcatatggcgctgaatgacctgtgcattaag

SEQ ID NO: 23 - DNA, 35 bp, Artificial Sequence
  primer
aagcttctcgagtcattacagcggacgttcgcccc

(19) United States
(12) Patent Application Publication
(10) Pub. No.: US 2021/0292379 A1
(43) Pub. Date: Sep. 23, 2021

(54) METHOD FOR PRODUCING DI-CHAIN CLOSTRIDIAL NEUROTOXINS

(71) Applicant: Ipsen Biopharm Limited, Wrexham (GB)

(72) Inventor: Daniel KWAN, Abingdon (GB)

(21) Appl. No.: 16/324,892

(22) PCT Filed: Sep. 13, 2017
(86) PCT No.: PCT/EP2017/073030

§ 371 Date: Feb. 11, 2019

(30) Foreign Application Priority Data
Sep. 16, 2016 (EP) 16189221.1

(51) Int. Cl. C07K 14/33; C12N 15/70

(52) U.S. Cl. CPC C07K 14/33; A61K 38/00; C12N 15/70

(57) ABSTRACT

The present invention provides methods, cells and kits suitable for recombinant production of di-chain clostridial neurotoxins, which avoid the requirement of an activation step, as well as di-chain clostridial neurotoxins thereby obtained which are suitable for use in therapy.

### FIELD OF THE INVENTION

The present invention provides a method for recombinant production of di-chain clostridial neurotoxins, which avoids the requirement of an activation step.

### BACKGROUND OF THE INVENTION

Bacteria in the genus Clostridia produce highly potent and specific protein toxins, which can poison neurons and other cells to which they are delivered. Examples of such clostridial toxins include the neurotoxins produced by C. tetani (TeNT) and by C. botulinum (BoNT) serotypes A-G, as well as those produced by C. baratii and C. butyricum.

Among the clostridial neurotoxins are some of the most potent toxins known. By way of example, botulinum neurotoxins have median lethal dose (LD50) values for mice ranging from 0.5 to 5 ng/kg, depending on the serotype. Both tetanus and botulinum toxins act by inhibiting the function of affected neurons, specifically the release of neurotransmitters. While botulinum toxin acts at the neuromuscular junction and inhibits cholinergic transmission in the peripheral nervous system, tetanus toxin acts in the central nervous system.

Clostridial neurotoxins act by proteolytically cleaving intracellular transport proteins known as SNARE proteins (e.g. SNAP-25, VAMP, or Syntaxin)—see Gerald K (2002) “Cell and Molecular Biology” (4th edition) John Wiley & Sons, Inc. The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins. Accordingly, once delivered to a desired target cell, the non-cytotoxic protease is capable of inhibiting cellular secretion from the target cell.

In nature, clostridial neurotoxins are synthesised as a single-chain polypeptide that is modified post-translationally by a proteolytic cleavage event to form two polypeptide chains joined together by a disulphide bond. Cleavage occurs at a specific cleavage site, often referred to as the activation site, which is located between the cysteine residues that provide the inter-chain disulphide bond. It is only through this activation event that full potency of the clostridial neurotoxin is achieved. The two chains are termed the heavy chain (H-chain), which has a molecular mass of approximately 100 kDa, and the light chain (L-chain), which has a molecular mass of approximately 50 kDa. The H-chain comprises an N-terminal translocation component (H_{N} domain) and a C-terminal targeting component (H_{C} domain). The cleavage site is located between the L-chain and the translocation domain components. Following binding of the H_{C} domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease).

Botulinum neurotoxins are well known for their ability to cause a flaccid muscle paralysis and inhibit cholinergic secretions. These properties have led to botulinum neurotoxins being employed in a variety of medical and cosmetic procedures, including treatment of glabellar lines or hyperkinetic facial lines, headache, hemifacial spasm, hyperactivity of the bladder, hyperhidrosis, nasal labial lines, cervical dystonia, blepharospasm, spasticity and hyperhidrosis.

Currently all approved drugs/cosmetic preparations comprising BoNTs contain naturally occurring neurotoxins, purified from clostridial strains (BoNT/A in the case of DYSPORT®, BOTOX® or XEOMIN®, and BoNT/B in the case of MYOBLOC®). The traditional production of BoNT products is carried out by culture of C. botulinum, followed by isolation and purification of the botulinum neurotoxin complex or complex free neurotoxin. C. botulinum are spore-forming bacteria and therefore require special culture equipment and facilities, which are cumbersome. Recombinant production of BoNT in a heterologous host such as E. coli, would therefore be advantageous. However, a limiting step of recombinant manufacture of clostridial neurotoxins is the activation step.

Indeed, current practice for recombinant clostridial neurotoxin manufacture is to express the clostridial neurotoxin as a single polypeptide chain in a suitable heterologous host such as E. coli (upstream process). This initial step is usually followed by a series of purification steps (eg by chromatography) and an activation step requiring the addition of a suitable protease which converts the single chain inactive (or hardly active) clostridial neurotoxin into a di-chain fully active form (downstream process). The activation step requires a specific and controlled cleavage of the clostridial neurotoxin activation loop. This cleavage is achieved by using a suitable protease to produce the desired di-chain clostridial neurotoxin, comprising a light chain and a heavy linked by a disulfide bond. This activation step has proved a very challenging stage of clostridial neurotoxin production. In particular, cleavage events can occur outside the activation loop and lead to the generation of truncated clostridial neurotoxins which must then be separated from the full length di-chain clostridial neurotoxins. In addition, following an incubation period the activating protease has to be removed from the activated toxin in order to avoid contaminating the final pharmaceutical product.

Issues that can be encountered at the activation stage include:
The difficulty in identifying a protease that will cleave the activation loop while avoiding unwanted cleavage at other sites (leading to truncation and reduction/loss in potency);
The difficulty in removing the activating protease from the final product;
The difficulty in sourcing a GMP grade activating protease for the manufacture of development and commercial products;
The time-consuming approach to determine the best activation conditions (temperature, time, ratio activating enzyme/neurotoxin . . . ).

A method for recombinant manufacture of clostridial neurotoxins which would bypass the requirement for an activation step would therefore be of great benefit.

Maisey et al. 1988 (MAISEY, E. Anne, et al. “Involvement of the constituent chains of botulinum neurotoxins A and B in the blockade of neurotransmitter release.” European Journal of Biochemistry 177.3 (1988): 683-691.) attempted to form di-chain BoNT/A and B using previously purified toxin that had been unfolded with the resulting domains refolded separately. When the separate domains where combined they found >70% of toxin did form di-chain toxin however potency was greatly reduced. In their discussion they suggest that this reduced potency is likely to be attributed to the presence of the free domains, non-covalent associations or incorrect disulfide formation.

US2006/0024794 A1 addresses the possibility of co-expressing BoNT domains to produce a di-chain toxin in insect cells using a baclovirus expression system. However, the data presented in particular in FIGS. 10 and 11 of US2006/0024794 A1 show that although a small proportion of di-chain neurotoxin is formed the majority of the clostridial neurotoxin remains as free light chain and heavy chain.

There is therefore a need in the art for improved methods for the recombinant production of di-chain clostridial neurotoxins.

### SUMMARY OF THE INVENTION

In a first aspect, the present invention provides a method for producing a di-chain clostridial neurotoxin, comprising separately expressing in a heterologous host cell a first gene encoding a clostridial neurotoxin light chain and a second gene encoding a clostridial neurotoxin heavy chain, wherein said first and second genes are expressed in an oxidizing environment of said host cell.

In a second aspect, the present invention provides a cell comprising a first gene encoding a clostridial neurotoxin light chain, and a second gene encoding a clostridial neurotoxin heavy chain, wherein said first and second genes are expressed in an oxidizing environment of said cell.

In a third aspect, the present invention provides a kit comprising
a. a cell comprising an oxidizing environment,
b. a first gene encoding a clostridial neurotoxin light chain, and
c. a second gene encoding a clostridial neurotoxin heavy chain,
wherein said first and second genes are suitable for separately expressing a clostridial neurotoxin light and a heavy chain in said oxidizing environment of said cell.

In a fourth aspect, the present invention provides a di-chain clostridial neurotoxin obtained by the method according to the invention.

In a fifth aspect, the present invention provides a pharmaceutical composition comprising a di-chain clostridial neurotoxin according to the invention.

In a sixth aspect, the present invention provides the use of a host cell which has an oxidative cytoplasm for producing a di-chain clostridial neurotoxin, wherein said host cell comprises a first gene encoding a clostridial neurotoxin light chain and a second gene encoding a clostridial neurotoxin heavy chain, wherein said first and second genes are expressed in the oxidative cytoplasm of said host cell.

### DETAILED DESCRIPTION OF THE INVENTION

The present invention is based on the finding by the inventors that co-expressing clostridial neurotoxin light and heavy chains separately within an oxidizing environment of a heterologous host cell, allows the two domains to fold together to form a di-chain clostridial neurotoxin with a drastically increased efficiency.

In a first aspect, the present invention provides a method for producing a di-chain clostridial neurotoxin, comprising separately expressing in a heterologous host cell a first gene encoding a clostridial neurotoxin light chain and a second gene encoding a clostridial neurotoxin heavy chain, wherein the first and second genes are expressed in an oxidizing environment of said host cell.

The term “oxidizing environment” as used herein means a cellular environment that promotes cystine formation (oxidised dimer of cysteine). This is generally achieved through the balance of differing redox proteins such as but not limited to thioredoxin based proteins (e.g DsbA) and glutathione. Non-limiting examples of oxidising environments are the periplasm of Gram negative bacteria or the endoplasmic reticulum of eukaryotic expression systems such as Chinese hamster ovary (CHO), insect or yeast cells.

Numerous prokaryotic and eukaryotic expression systems are known in the state of the art. The host cell can be selected, for example, from prokaryotic cells such as Escherichia coli and Bacillus megaterium, or from eukaryotic cells such as Saccharomyces cerevisiae and Pichia pastoris. Although higher eukaryotic cells, such as insect cells or mammal cells, may be used as well, host cells are nevertheless preferred, which, like C. botulinum, do not possess glycosylation apparatus.

In a preferred embodiment, the host cell is a prokaryote cell. In a more preferred embodiment, the oxidizing environment is the cytoplasm of the prokaryote cell.

Disulfide bonds are formed by the oxidation of sulfhydryl groups between two cysteine side chains resulting in a covalent bond. In nature, cells have enzymes dedicated to reducing disulfide bonds in their cytoplasm (reducing cytoplasm) and the formation of disulphide bonds occurs in extra-cytoplasmic environments such as the periplasm in gram negative bacteria or the endoplasmic reticulum (ER) in eukaryotes. Therefore, production of recombinant proteins requiring disulfide bonds in the cytoplasm of cells such as E. coli is challenging.

The cytoplasm of bacterial cells can be rendered oxidizing through genetic engineering, eg by expressing in the cytoplasm genes involved in disulphide bond formation and/or repressing genes involved in disulphide bond reduction and/or modifying such genes. For example, introducing mutations into genes of the thioredoxin (trxB) and/or glutathione (gor or gshA) pathways and/or cytoplasmically over-expressing DsbC can render the cytoplasmic environment oxidadizing and allow for the formation of disulphide bonds (Bessette, Paul H., et al. “Efficient folding of proteins with multiple disulfide bonds in the Escherichia coli cytoplasm.” Proceedings of the National Academy of Sciences 96.24 (1999): 13703-13708; Lobstein, Julie, et al. “SHuffle, a novel Escherichia coli protein expression strain capable of correctly folding disulfide bonded proteins in its cytoplasm.” Microbial cell factories 11.1 (2012): 1).

Examples of commercially available E. coli strains with oxidizing environment include:
AD494 and BL21trxB strains, available from Novagen, in which the trxB gene is mutated;
Origami™ strains (Origami, Origami 2, Origami B) available from Novagen, in which the gor and trxB genes are mutated;
Rosetta-gami™ strains (Rosetta-gami, Rosetta-gami 2 and Rosetta-gami B) available from Novagen, in which the gor and trxB genes are mutated;
SHuffle® strains (SHuffle T7, SHuffle T7 express) available from New England Biolabs, in which the gor and trxB genes are mutated and a DsbC gene lacking its signal sequence is expressed in the cytoplasm.

In a preferred embodiment, the cell is a prokaryote cell in which at least one gene involved in disulphide bond formation is overexpressed in the cytoplasm as compared to an otherwise identical wild-type cell and/or at least one gene involved in disulphide bond reduction is repressed as compared to an otherwise identical wild-type cell. In one embodiment, the prokaryote cell is an E. coli cell from a strain selected from AD494, BL21trxB, Origami, Rosetta-gami and SHuffle strains. In a preferred embodiment, the prokaryote cell is an E. coli cell from a Origami or SHuffle strain.

The term “neurotoxin” as used herein means any polypeptide that enters a neuron and inhibits neurotransmitter release. This process encompasses the binding of the neurotoxin to a low or high affinity receptor, the internalisation of the neurotoxin, the translocation of the endopeptidase portion of the neurotoxin into the cytoplasm and the enzymatic modification of the neurotoxin substrate. More specifically, the term “neurotoxin” encompasses any polypeptide produced by Clostridium bacteria (“clostridial neurotoxins”) that enters a neuron and inhibits neurotransmitter release, and such polypeptides produced by recombinant technologies or chemical techniques. It is this di-chain form that is the active form of the toxin. The two chains are termed the heavy chain (H-chain), which has a molecular mass of approximately 100 kDa, and the light chain (L-chain), which has a molecular mass of approximately 50 kDa. The L-chain comprises the endopeptidase activity. The H-chain comprises two functionally distinct domains each having molecular weight of approximately 50 kDa: the “H_{C} domain” that enables the binding of the neurotoxin to a receptor located on the surface of the target cell, and the “H_{N} domain” that enables translocation of the light chain (endopeptidase) into the cytoplasm. The H_{C} domain consists of two structurally distinct subdomains, the “H_{CN} subdomain” (N-terminal part of the H_{C} domain) and the “H_{CC} subdomain” (C-terminal part of the H_{C} domain), each of which has a molecular weight of approximately 25 kDa. The term “di-chain clostridial neurotoxin” as used herein means an active neurotoxin consisting of a clostridial neurotoxin light chain and heavy chain which are linked by a disulphide bond. It is understood that a di-chain clostridial neurotoxin according to the invention is capable of binding to a target cell, of translocating the light chain into the cytoplasm of the target cell and of cleaving a SNARE protein, thereby impairing the target's cell's secretion ability.

Different botulinum neurotoxin (BoNT) serotypes can be distinguished based on inactivation by specific neutralising anti-sera, with such classification by serotype correlating with percentage sequence identity at the amino acid level. BoNT proteins of a given serotype are further divided into different subtypes on the basis of amino acid percentage sequence identity. An example of a BoNT/A amino acid sequence is provided as SEQ ID NO: 1 (UniProt accession number A5HZZ9). An example of a BoNT/B amino acid sequence is provided as SEQ ID NO: 2 (UniProt accession number B1INP5). An example of a BoNT/C amino acid sequence is provided as SEQ ID NO: 3 (UniProt accession number P18640). An example of a BoNT/D amino acid sequence is provided as SEQ ID NO: 4 (UniProt accession number P19321). An example of a BoNT/E amino acid sequence is provided as SEQ ID NO: 5 (accession number WP_003372387). An example of a BoNT/F amino acid sequence is provided as SEQ ID NO: 6 (UniProt accession number Q57236). An example of a BoNT/G amino acid sequence is provided as SEQ ID NO: 7 (accession number WP_039635782). An example of a Tetanus neurotoxin (TeNT) amino acid sequence is provided as SEQ ID NO: 8 (UniProt accession number P04958).

An example of a nucleic acid sequence encoding a BoNT/A is provided as SEQ ID NO: 9. An a nucleic acid sequence encoding a BoNT/B is provided as SEQ ID NO: 10. An a nucleic acid sequence encoding a BoNT/C is provided as SEQ ID NO: 11. An a nucleic acid sequence encoding a BoNT/D is provided as SEQ ID NO: 12. An a nucleic acid sequence encoding a BoNT/E is provided as SEQ ID NO: 13. An a nucleic acid sequence encoding a BoNT/F is provided as SEQ ID NO: 14. An a nucleic acid sequence encoding a BoNT/G sequence is provided as SEQ ID NO: 15. An a nucleic acid sequence encoding a Tetanus neurotoxin (TeNT) sequence is provided as SEQ ID NO: 16.

Exemplary L, H_{N}, H_{CN} and H_{CC} amino acid domains are shown in table 1.

TABLE 1
Exemplary amino acid L, H_{N}, H_{C}, H_{CN} and H_{CC} domains

| Neurotoxin | Accession Number | SEQ ID NO | L | H_{N} | H_{CN} | H_{CC} |
|---|---|---|---|---|---|---|
| BoNT/A1 | A5HZZ9 | 1 | 1-448 | 449-872 | 873-1094 | 1095-1296 |
| BoNT/B1 | B1INP5 | 2 | 1-441 | 442-859 | 860-1081 | 1082-1291 |
| BoNT/C1 | P18640 | 3 | 1-449 | 450-867 | 868-1095 | 1096-1291 |
| BoNT/D | P19321 | 4 | 1-442 | 443-863 | 864-1082 | 1083-1276 |
| BoNT/E1 | WP_003372387 | 5 | 1-423 | 424-846 | 847-1069 | 1070-1252 |
| BoNT/F1 | Q57236 | 6 | 1-439 | 440-865 | 866-1087 | 1088-1278 |
| BoNT/G | WP_039635782 | 7 | 1-446 | 447-864 | 865-1089 | 1090-1297 |
| TeNT | P04958 | 8 | 1-456 | 457-880 | 881-1111 | 1112-1315 |

Exemplary nucleic acid sequences encoding L, H_{N}, H_{CN} and H_{CC} domains are shown in table 2.

TABLE 2
Exemplary nucleic acid sequences encoding
L, H_{N}, H_{C}, H_{CN} and H_{CC} domains

| Neurotoxin | SEQ ID NO | L | H_{N} | H_{CN} | H_{CC} |
|---|---|---|---|---|---|
| BoNT/A1 | 9 | 1-1344 | 1345-2616 | 2617-3282 | 3283-3888 |
| BoNT/B1 | 10 | 1-1323 | 1324-2577 | 2578-3243 | 3244-3873 |
| BoNT/C1 | 11 | 1-1347 | 1348-2601 | 2602-3285 | 3286-3873 |
| BoNT/D | 12 | 1-1326 | 1327-2589 | 2590-3246 | 3247-3828 |
| BoNT/E1 | 13 | 1-1269 | 1270-2538 | 2539-3207 | 3208-3756 |
| BoNT/F1 | 14 | 1-1317 | 1318-2595 | 2596-3261 | 3262-3834 |
| BoNT/G | 15 | 1-1338 | 1339-2592 | 2593-3267 | 3268-3891 |
| TeNT | 16 | 1-1368 | 1369-2640 | 2641-3333 | 3334-3945 |

The above-identified reference sequences should be considered a guide, as slight variations may occur according to sub-serotypes. By way of example, US 2007/0166332 (hereby incorporated by reference in its entirety) cites slightly different clostridial sequences”.

In one embodiment, the clostridial neurotoxin light chain is from a BoNT type A, type B, type C1, type D, type E, type F or type G, or a TeNT.

In one embodiment, the clostridial neurotoxin heavy chain is from a BoNT type A, type B, type C1, type D, type E, type F or type G, or a TeNT.

In one embodiment, the clostridial neurotoxin light chain is from a BoNT type A, type B, type C1, type D, type E, type F or type G, or a TeNT, and the clostridial neurotoxin heavy chain is from a BoNT type A, type B, type C1, type D, type E, type F or type G, or a TeNT.

In one embodiment, the clostridial neurotoxin light and heavy chains are from the same serotype or subtype.

In one embodiment, the clostridial neurotoxin light and heavy chains are from different serotypes or subtypes.

In one embodiment, the clostridial neurotoxin light chain comprises a sequence selected from:
amino acid residues 1 to 448 of SEQ ID NO: 1, or a polypeptide sequence having at least 70%, preferably at least 75%, 80%, 85%, 90%, 95% or 99% sequence identity thereto,
amino acid residues 1 to 441 of SEQ ID NO: 2, or a polypeptide sequence having at least 70%, preferably at least 75%, 80%, 85%, 90%, 95% or 99% sequence identity thereto,
amino acid residues 1 to 449 of SEQ ID NO: 3, or a polypeptide sequence having at least 70%, preferably at least 75%, 80%, 85%, 90%, 95% or 99% sequence identity thereto,
amino acid residues 1 to 442 of SEQ ID NO: 4, or a polypeptide sequence having at least 70%, preferably at least 75%, 80%, 85%, 90%, 95% or 99% sequence identity thereto,
amino acid residues 1 to 423 of SEQ ID NO: 5, or a polypeptide sequence having at least 70%, preferably at least 75%, 80%, 85%, 90%, 95% or 99% sequence identity thereto,
amino acid residues 1 to 439 of SEQ ID NO: 6, or a polypeptide sequence having at least 70%, preferably at least 75%, 80%, 85%, 90%, 95% or 99% sequence identity thereto,
amino acid residues 1 to 446 of SEQ ID NO: 7, or a polypeptide sequence having at least 70%, preferably at least 75%, 80%, 85%, 90%, 95% or 99% sequence identity thereto G,
amino acid residues 1 to 456 of SEQ ID NO: 8, or a polypeptide sequence having at least 70%, preferably at least 75%, 80%, 85%, 90%, 95% or 99% sequence identity thereto,
an amino acid sequence encoded by nucleotides 1 to 1344 of SEQ ID NO: 9, or by a nucleic acid sequence having at least 70%, preferably at least 75%, 80%, 85%, 90%, 95% or 99% sequence identity thereto,
an amino acid sequence encoded by nucleotides 1 to 1323 of SEQ ID NO: 10, or by a nucleic acid sequence having at least 70%, preferably at least 75%, 80%, 85%, 90%, 95% or 99% sequence identity thereto,
an amino acid sequence encoded by nucleotides 1 to 1347 of SEQ ID NO: 11, or by a nucleic acid sequence having at least 70%, preferably at least 75%, 80%, 85%, 90%, 95% or 99% sequence identity thereto,
an amino acid sequence encoded by nucleotides 1 to 1326 of SEQ ID NO: 12, or by a nucleic acid sequence having at least 70%, preferably at least 75%, 80%, 85%, 90%, 95% or 99% sequence identity thereto,
an amino acid sequence encoded by nucleotides 1 to 1269 of SEQ ID NO: 13, or by a nucleic acid sequence having at least 70%, preferably at least 75%, 80%, 85%, 90%, 95% or 99% sequence identity thereto,
an amino acid sequence encoded by nucleotides 1 to 1317 of SEQ ID NO: 14, or by a nucleic acid sequence having at least 70%, preferably at least 75%, 80%, 85%, 90%, 95% or 99% sequence identity thereto,
an amino acid sequence encoded by nucleotides 1 to 1338 of SEQ ID NO: 15, or by a nucleic acid sequence having at least 70%, preferably at least 75%, 80%, 85%, 90%, 95% or 99% sequence identity thereto, and
an amino acid sequence encoded by nucleotides 1 to 1368 of SEQ ID NO: 16, or by a nucleic acid sequence having at least 70%, preferably at least 75%, 80%, 85%, 90%, 95% or 99% sequence identity thereto.

It is understood that a clostridial neurotoxin light chain is capable of cleaving a SNARE protein.

In one embodiment, the clostridial neurotoxin heavy chain comprises a sequence selected from:
amino acid residues 449 to 1296 of SEQ ID NO: 1, or a polypeptide sequence having at least 70%, preferably at least 75%, 80%, 85%, 90%, 95% or 99% sequence identity thereto,
amino acid residues 442 to 1291 of SEQ ID NO: 2, or a polypeptide sequence having at least 70%, preferably at least 75%, 80%, 85%, 90%, 95% or 99% sequence identity thereto,
amino acid residues 450 to 1291 of SEQ ID NO: 3, or a polypeptide sequence having at least 70%, preferably at least 75%, 80%, 85%, 90%, 95% or 99% sequence identity thereto,
amino acid residues 443 to 1276 of SEQ ID NO: 4, or a polypeptide sequence having at least 70%, preferably at least 75%, 80%, 85%, 90%, 95% or 99% sequence identity thereto,
amino acid residues 424 to 1252 of SEQ ID NO: 5, or a polypeptide sequence having at least 70%, preferably at least 75%, 80%, 85%, 90%, 95% or 99% sequence identity thereto,
amino acid residues 440 to 1278 of SEQ ID NO: 6, or a polypeptide sequence having at least 70%, preferably at least 75%, 80%, 85%, 90%, 95% or 99% sequence identity thereto,
amino acid residues 447 to 1297 of SEQ ID NO: 7, or a polypeptide sequence having at least 70%, preferably at least 75%, 80%, 85%, 90%, 95% or 99% sequence identity thereto,
amino acid residues 457 to 1315 of SEQ ID NO: 8, or a polypeptide sequence having at least 70%, preferably at least 75%, 80%, 85%, 90%, 95% or 99% sequence identity thereto,
an amino acid sequence encoded by nucleotides 1345 to 3888 of SEQ ID NO: 9, or by a nucleic acid sequence having at least 70%, preferably at least 75%, 80%, 85%, 90%, 95% or 99% sequence identity thereto,
an amino acid sequence encoded by nucleotides 1324 to 3873 of SEQ ID NO: 10, or by a nucleic acid sequence having at least 70%, preferably at least 75%, 80%, 85%, 90%, 95% or 99% sequence identity thereto,
an amino acid sequence encoded by nucleotides 1348 to 3873 of SEQ ID NO: 11, or by a nucleic acid sequence having at least 70%, preferably at least 75%, 80%, 85%, 90%, 95% or 99% sequence identity thereto,
an amino acid sequence encoded by nucleotides 1327 to 3828 of SEQ ID NO: 12, or by a nucleic acid sequence having at least 70%, preferably at least 75%, 80%, 85%, 90%, 95% or 99% sequence identity thereto,
an amino acid sequence encoded by nucleotides 1270 to 3756 of SEQ ID NO: 13, or by a nucleic acid sequence having at least 70%, preferably at least 75%, 80%, 85%, 90%, 95% or 99% sequence identity thereto,
an amino acid sequence encoded by nucleotides 1318 to 3834 of SEQ ID NO: 14, or by a nucleic acid sequence having at least 70%, preferably at least 75%, 80%, 85%, 90%, 95% or 99% sequence identity thereto,
an amino acid sequence encoded by nucleotides 1339 to 3891 of SEQ ID NO: 15, or by a nucleic acid sequence having at least 70%, preferably at least 75%, 80%, 85%, 90%, 95% or 99% sequence identity thereto, and
an amino acid sequence encoded by nucleotides 1369 to 3945 of SEQ ID NO: 16, or by a nucleic acid sequence having at least 70%, preferably at least 75%, 80%, 85%, 90%, 95% or 99% sequence identity thereto.

It is understood that a clostridial neurotoxin heavy chain is capable of binding to a target cell and of translocating the light chain into the cytoplasm of the target cell.

It is also understood that the H_{N}, H_{CN} and H_{CC} domains of the clostridial neurotoxin heavy chain according to the invention can be from the same or from different clostridial serotypes or subtypes.

In one embodiment, the clostridial neurotoxin heavy chain comprises a H_{N}, a H_{CN} and a H_{CC} domain, wherein
the H_{N} domain comprises a sequence selected from:
   amino acid residues 449 to 872 of SEQ ID NO: 1, or a polypeptide sequence having at least 70%, preferably at least 75%, 80%, 85%, 90%, 95% or 99% sequence identity thereto,
   amino acid residues 442 to 859 of SEQ ID NO: 2, or a polypeptide sequence having at least 70%, preferably at least 75%, 80%, 85%, 90%, 95% or 99% sequence identity thereto,
   amino acid residues 450 to 867 of SEQ ID NO: 3, or a polypeptide sequence having at least 70%, preferably at least 75%, 80%, 85%, 90%, 95% or 99% sequence identity thereto,
   amino acid residues 443 to 863 of SEQ ID NO: 4, or a polypeptide sequence having at least 70%, preferably at least 75%, 80%, 85%, 90%, 95% or 99% sequence identity thereto,
   amino acid residues 424 to 846 of SEQ ID NO: 5, or a polypeptide sequence having at least 70%, preferably at least 75%, 80%, 85%, 90%, 95% or 99% sequence identity thereto,
   amino acid residues 440 to 865 of SEQ ID NO: 6, or a polypeptide sequence having at least 70%, preferably at least 75%, 80%, 85%, 90%, 95% or 99% sequence identity thereto,
   amino acid residues 447 to 864 of SEQ ID NO: 7, or a polypeptide sequence having at least 70%, preferably at least 75%, 80%, 85%, 90%, 95% or 99% sequence identity thereto,
   amino acid residues 457 to 880 of SEQ ID NO: 8, or a polypeptide sequence having at least 70%, preferably at least 75%, 80%, 85%, 90%, 95% or 99% sequence identity thereto,
   an amino acid sequence encoded by nucleotides 1345 to 2616 of SEQ ID NO: 9, or by a nucleic acid sequence having at least 70%, preferably at least 75%, 80%, 85%, 90%, 95% or 99% sequence identity thereto,
   an amino acid sequence encoded by nucleotides 1324 to 2577 of SEQ ID NO: 10, or by a nucleic acid sequence having at least 70%, preferably at least 75%, 80%, 85%, 90%, 95% or 99% sequence identity thereto,
   an amino acid sequence encoded by nucleotides 1348 to 2601 of SEQ ID NO: 11, or by a nucleic acid sequence having at least 70%, preferably at least 75%, 80%, 85%, 90%, 95% or 99% sequence identity thereto,
   an amino acid sequence encoded by nucleotides 1327 to 2589 of SEQ ID NO: 12, or by a nucleic acid sequence having at least 70%, preferably at least 75%, 80%, 85%, 90%, 95% or 99% sequence identity thereto,
   an amino acid sequence encoded by nucleotides 1270 to 2538 of SEQ ID NO: 13, or by a nucleic acid sequence having at least 70%, preferably at least 75%, 80%, 85%, 90%, 95% or 99% sequence identity thereto,
   an amino acid sequence encoded by nucleotides 1318 to 2595 of SEQ ID NO: 14, or by a nucleic acid sequence having at least 70%, preferably at least 75%, 80%, 85%, 90%, 95% or 99% sequence identity thereto,
   an amino acid sequence encoded by nucleotides 1339 to 2592 of SEQ ID NO: 15, or by a nucleic acid sequence having at least 70%, preferably at least 75%, 80%, 85%, 90%, 95% or 99% sequence identity thereto, and
   an amino acid sequence encoded by nucleotides 1369 to 2640 of SEQ ID NO: 16, or by a nucleic acid sequence having at least 70%, preferably at least 75%, 80%, 85%, 90%, 95% or 99% sequence identity thereto;
the H_{CN} domain comprises a sequence selected from:
   amino acid residues 873 to 1094 of SEQ ID NO: 1, or a polypeptide sequence having at least 70%, preferably at least 75%, 80%, 85%, 90%, 95% or 99% sequence identity thereto,
   amino acid residues 860 to 1081 of SEQ ID NO: 2, or a polypeptide sequence having at least 70%, preferably at least 75%, 80%, 85%, 90%, 95% or 99% sequence identity thereto,
   amino acid residues 868 to 1095 of SEQ ID NO: 3, or a polypeptide sequence having at least 70%, preferably at least 75%, 80%, 85%, 90%, 95% or 99% sequence identity thereto,
   amino acid residues 864 to 1082 of SEQ ID NO: 4, or a polypeptide sequence having at least 70%, preferably at least 75%, 80%, 85%, 90%, 95% or 99% sequence identity thereto,
   amino acid residues 847 to 1069 of SEQ ID NO: 5, or a polypeptide sequence having at least 70%, preferably at least 75%, 80%, 85%, 90%, 95% or 99% sequence identity thereto,
   amino acid residues 866 to 1087 of SEQ ID NO: 6, or a polypeptide sequence having at least 70%, preferably at least 75%, 80%, 85%, 90%, 95% or 99% sequence identity thereto,
   amino acid residues 865 to 1089 of SEQ ID NO: 7, or a polypeptide sequence having at least 70%, preferably at least 75%, 80%, 85%, 90%, 95% or 99% sequence identity thereto,
   amino acid residues 881 to 1111 of SEQ ID NO: 8, or a polypeptide sequence having at least 70%, preferably at least 75%, 80%, 85%, 90%, 95% or 99% sequence identity thereto,
   an amino acid sequence encoded by nucleotides 2617 to 3282 of SEQ ID NO: 9, or by a nucleic acid sequence having at least 70%, preferably at least 75%, 80%, 85%, 90%, 95% or 99% sequence identity thereto,
   an amino acid sequence encoded by nucleotides 2578 to 3243 of SEQ ID NO: 10, or by a nucleic acid sequence having at least 70%, preferably at least 75%, 80%, 85%, 90%, 95% or 99% sequence identity thereto,
   an amino acid sequence encoded by nucleotides 2602 to 3285 of SEQ ID NO: 11, or by a nucleic acid sequence having at least 70%, preferably at least 75%, 80%, 85%, 90%, 95% or 99% sequence identity thereto,
   an amino acid sequence encoded by nucleotides 2590 to 3246 of SEQ ID NO: 12, or by a nucleic acid sequence having at least 70%, preferably at least 75%, 80%, 85%, 90%, 95% or 99% sequence identity thereto,
   an amino acid sequence encoded by nucleotides 2539 to 3207 of SEQ ID NO: 13, or by a nucleic acid sequence having at least 70%, preferably at least 75%, 80%, 85%, 90%, 95% or 99% sequence identity thereto,
   an amino acid sequence encoded by nucleotides 2596 to 3261 of SEQ ID NO: 14, or by a nucleic acid sequence having at least 70%, preferably at least 75%, 80%, 85%, 90%, 95% or 99% sequence identity thereto,
   an amino acid sequence encoded by nucleotides 2593 to 3267 of SEQ ID NO: 15, or by a nucleic acid sequence having at least 70%, preferably at least 75%, 80%, 85%, 90%, 95% or 99% sequence identity thereto, and
   an amino acid sequence encoded by nucleotides 2641 to 3333 of SEQ ID NO: 16, or by a nucleic acid sequence having at least 70%, preferably at least 75%, 80%, 85%, 90%, 95% or 99% sequence identity thereto;
and the H_{CC} domain comprises a sequence selected from:
   amino acid residues 1095 to 1296 of SEQ ID NO: 1, or a polypeptide sequence having at least 70%, preferably at least 75%, 80%, 85%, 90%, 95% or 99% sequence identity thereto,
   amino acid residues 1082 to 1291 of SEQ ID NO: 2, or a polypeptide sequence having at least 70%, preferably at least 75%, 80%, 85%, 90%, 95% or 99% sequence identity thereto,
   amino acid residues 1096 to 1291 of SEQ ID NO: 3, or a polypeptide sequence having at least 70%, preferably at least 75%, 80%, 85%, 90%, 95% or 99% sequence identity thereto,
   amino acid residues 1083 to 1276 of SEQ ID NO: 4, or a polypeptide sequence having at least 70%, preferably at least 75%, 80%, 85%, 90%, 95% or 99% sequence identity thereto,
   amino acid residues 1070 to 1252 of SEQ ID NO: 5, or a polypeptide sequence having at least 70%, preferably at least 75%, 80%, 85%, 90%, 95% or 99% sequence identity thereto,
   amino acid residues 1088 to 1278 of SEQ ID NO: 6, or a polypeptide sequence having at least 70%, preferably at least 75%, 80%, 85%, 90%, 95% or 99% sequence identity thereto,
   amino acid residues 1090 to 1297 of SEQ ID NO: 7, or a polypeptide sequence having at least 70%, preferably at least 75%, 80%, 85%, 90%, 95% or 99% sequence identity thereto,
   amino acid residues 1112 to 1315 of SEQ ID NO: 8, or a polypeptide sequence having at least 70%, preferably at least 75%, 80%, 85%, 90%, 95% or 99% sequence identity thereto,
   an amino acid sequence encoded by nucleotides 3283 to 3888 of SEQ ID NO: 9, or by a nucleic acid sequence having at least 70%, preferably at least 75%, 80%, 85%, 90%, 95% or 99% sequence identity thereto,
   an amino acid sequence encoded by nucleotides 3244 to 3873 of SEQ ID NO: 10, or by a nucleic acid sequence having at least 70%, preferably at least 75%, 80%, 85%, 90%, 95% or 99% sequence identity thereto,
   an amino acid sequence encoded by nucleotides 3286 to 3873 of SEQ ID NO: 11, or by a nucleic acid sequence having at least 70%, preferably at least 75%, 80%, 85%, 90%, 95% or 99% sequence identity thereto,
   an amino acid sequence encoded by nucleotides 3247 to 3828 of SEQ ID NO: 12, or by a nucleic acid sequence having at least 70%, preferably at least 75%, 80%, 85%, 90%, 95% or 99% sequence identity thereto,
   an amino acid sequence encoded by nucleotides 3208 to 3756 of SEQ ID NO: 13, or by a nucleic acid sequence having at least 70%, preferably at least 75%, 80%, 85%, 90%, 95% or 99% sequence identity thereto,
   an amino acid sequence encoded by nucleotides 3262 to 3834 of SEQ ID NO: 14, or by a nucleic acid sequence having at least 70%, preferably at least 75%, 80%, 85%, 90%, 95% or 99% sequence identity thereto,
   an amino acid sequence encoded by nucleotides 3268 to 3891 of SEQ ID NO: 15, or by a nucleic acid sequence having at least 70%, preferably at least 75%, 80%, 85%, 90%, 95% or 99% sequence identity thereto, and
   an amino acid sequence encoded by nucleotides 3334 to 3945 of SEQ ID NO: 16, or by a nucleic acid sequence having at least 70%, preferably at least 75%, 80%, 85%, 90%, 95% or 99% sequence identity thereto.

The “percent sequence identity” between two or more nucleic acid or amino acid sequences is a function of the number of identical nucleotides/amino acids at identical positions shared by the aligned sequences. Thus, % identity may be calculated as the number of identical nucleotides/amino acids at each position in an alignment divided by the total number of nucleotides/amino acids in the aligned sequence, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

The light and/or heavy chains can be from a mosaic neurotoxin. The term “mosaic neurotoxin” as used in this context refers to a naturally occurring clostridial neurotoxin that comprises at least one functional domain from another type of clostridial neurotoxins (e.g. a clostridial neurotoxin of a different serotype), said clostridial neurotoxin not usually comprising said at least one functional domain. Examples of mosaic neurotoxins are naturally occurring BoNT/DC and BoNT/CD. BoNT/DC comprises the L chain and H_{N} domain of serotype D and the H_{C} domain of serotype C, whereas BoNT/CD consists of the L chain and H_{N} domain of serotype C and the H_{C} domain of serotype D.

The light and/or heavy chains can be from a modified neurotoxin and derivatives thereof, including but not limited to those described below. A modified neurotoxin or derivative may contain one or more amino acids that has been modified as compared to the native (unmodified) form of the neurotoxin, or may contain one or more inserted amino acids that are not present in the native (unmodified) form of the toxin. By way of example, a modified clostridial neurotoxin may have modified amino acid sequences in one or more domains relative to the native (unmodified) clostridial neurotoxin sequence. Such modifications may modify functional aspects of the neurotoxin, for example biological activity or persistence. Thus, in one embodiment, the first neurotoxin and/or the second neurotoxin is a modified neurotoxin, or modified neurotoxin derivative.

A modified neurotoxin retains at least one of the functions of a neurotoxin, selected from the ability to bind to a low or high affinity neurotoxin receptor on a target cell, to translocate the endopeptidase portion of the neurotoxin (light chain) into the cell cytoplasm and to cleave a SNARE protein. Preferably, a modified neurotoxin retains at least two of these functions. More preferably a modified neurotoxin retains these three functions.

A modified neurotoxin may have one or more modifications in the amino acid sequence of the heavy chain (such as a modified H_{C} domain), wherein said modified heavy chain binds to target nerve cells with a higher or lower affinity than the native (unmodified) neurotoxin. Such modifications in the H_{C} domain can include modifying residues in the ganglioside binding site of the H_{C} domain or in the protein (SV2 or synaptotagmin) binding site that alter binding to the ganglioside receptor and/or the protein receptor of the target nerve cell. Examples of such modified neurotoxins are described in WO 2006/027207 and WO 2006/114308, both of which are hereby incorporated by reference in their entirety.

A modified neurotoxin may have one or more modifications in the amino acid sequence of the light chain, for example modifications in the substrate binding or catalytic domain which may alter or modify the SNARE protein specificity of the modified LC. Examples of such modified neurotoxins are described in WO 2010/120766 and US 2011/0318385, both of which are hereby incorporated by reference in their entirety.

A modified neurotoxin may comprise one or more modifications that increases or decreases the biological activity and/or the biological persistence of the modified neurotoxin. For example, a modified neurotoxin may comprise a leucine- or tyrosine-based motif, wherein said motif increases or decreases the biological activity and/or the biological persistence of the modified neurotoxin. Suitable leucine-based motifs include xDxxxLL, xExxxLL, xExxxlL, and xExxxLM (wherein x is any amino acid). Suitable tyrosine-based motifs include Y-x-x-Hy (wherein Hy is a hydrophobic amino acid). Examples of modified neurotoxins comprising leucine- and tyrosine-based motifs are described in WO 2002/08268, which is hereby incorporated by reference in its entirety.

In one embodiment, the clostridial neurotoxin is a retargeted neurotoxin. The term “retargeted neurotoxin” (also referred to as “targeted secretion inhibitors”, “TSIs”, “TVEMPs” or “TEMs”) as used herein means a clostridial neurotoxin comprising a Targeting Moiety (TM) which binds to a non clostridial receptor. The TM can replace part or all of the H_{C} or H_{CC} domain of the clostridial neurotoxin heavy chain. Examples of retargeted neurotoxins are disclosed in WO96/33273, WO98/07864, WO00/10598, WO01/21213, WO01/53336; WO02/07759 WO2005/023309, WO2006/026780, WO2006/099590, WO2006/056093, WO2006/059105, WO2006/059113, WO2007/138339, WO2007/106115, WO2007/106799, WO2009/150469, WO2009/150470, WO2010/055358, WO2010/020811, WO2010/138379, WO2010/138395, WO2010/138382, WO2011/020052, WO2011/020056, WO2011/020114, WO2011/020117, WO2011/20119, WO2012/156743, WO2012/134900, WO2012/134897, WO2012/134904, WO2012/134902, WO2012/135343, WO2012/135448, WO2012/135304, WO2012/134902, WO2014/033441, WO2014/128497, WO2014/053651, WO2015/004464, all of which are herein incorporated by reference.

In one embodiment, the gene encoding a clostridial neurotoxin light chain and the gene encoding a clostridial neurotoxin heavy chain are present on the same vector.

In one embodiment, the gene encoding a clostridial neurotoxin light chain and the gene encoding a clostridial neurotoxin heavy chain are present on different vectors.

In principle, any expression vectors can be used to achieve co-expression in E. coli for example pK7, pJ401, pBAD or pET vectors. When using separate vectors to express each domain it is preferable to use different antibiotic resistance markers and origins of replication to help plasmid stability. With the single vector approach it is generally beneficial to have genes under control of separate promoters and ribosome binding sites but this is not essential. Finally, both strategies can control both genes by the same type of promoter or can utilise different ones for each e.g. a T7-lac, T5-lac, rhaBAD and araBAD promoter.

In one embodiment, the gene encoding a clostridial neurotoxin light chain and the gene encoding a clostridial neurotoxin heavy chain are prepared as part of DNA or RNA vector(s), preferably DNA vector(s), comprising a promoter and a terminator. Suitable promoter and terminator sequences are well known in the art.

The choice of promoter depends in this case on the expression systems used for expression. In general, constitutive promoters are preferred, but inducible promoters may likewise be used. The construct produced in this manner includes at least one part of a vector, in particular regulatory elements, the vector, for example, being selected from A-derivates, adenoviruses, baculoviruses, vaccinia viruses, SV40-viruses and retroviruses. The vector is preferably capable of expressing the genes in a given host cell.

In one embodiment, the vector has a promoter selected from:

| | | Typical |
|---|---|---|
| | Induction | Induction |
| Promoter | Agent | Condition |
| Tac (hybrid) | IPTG | 0.2 mM |
| | | (0.05-2.0 mM) |
| AraBAD | L-arabinose | 0.2% |
| | | (0.00002-0.4%) |
| T7-lac operator | IPTG | 0.2 mM |
| | | (0.05-2.0 mM) |
| T5-lac operator | IPTG | 0.2 mM |
| | | (0.05-2.0 mM) |

The genes of the invention may be made using any suitable process known in the art. Thus, the genes may be made using chemical synthesis techniques. Alternatively, the genes of the invention may be made using molecular biology techniques.

The genes of the present invention are preferably designed in silico, and then synthesised by conventional gene synthesis techniques.

The above-mentioned genes are optionally modified for codon-biasing according to the ultimate host cell (e.g. E. coli) expression system that is to be employed.

In one embodiment, the method according to the invention further comprises a step of recovering the di-chain clostridial neurotoxin from the host cell. In particular, the method may include a step of lysing the host cell to provide a host cell homogenate, and a step of isolating the di-chain clostridial toxin protein. In one embodiment, the method according to the invention may further comprise a step of introducing the gene encoding a clostridial neurotoxin light chain and a gene encoding a clostridial neurotoxin heavy chain into the host cell. For example, the genes of the invention may be introduced into the cell in the form of expression vector(s) as described herein.

Typically the di-chain clostridial neurotoxin is purified and/or concentrated after recovery from the host cell. Any suitable method(s) may be used for the recovery, purification and/or concentration of the di-chain clostridial neurotoxin. Standard techniques for recovery, purification and/or concentration are known in the art, for example chromatography methods and/or electrophoresis.

The di-chain clostridial neurotoxin may comprise one or more N-terminal and/or C-terminal located purification tags to assist in the purification of the polypeptide. Whilst any purification tag may be employed, the following are preferred: His-tag (e.g. 6×histidine), preferably as a C-terminal and/or N-terminal tag; MBP-tag (maltose binding protein), preferably as an N-terminal tag; GST-tag (glutathione-S-transferase), preferably as an N-terminal tag; His-MBP-tag, preferably as an N-terminal tag; GST-MBP-tag, preferably as an N-terminal tag; Thioredoxin-tag, preferably as an N-terminal tag; and/or CBD-tag (Chitin Binding Domain), preferably as an N-terminal tag.

One or more peptide spacer/linker molecules may be included in the di-chain clostridial neurotoxin. For example, a peptide spacer may be employed between a purification tag and the rest of the polypeptide molecule.

In a further aspect, the present invention provides a cell comprising a first genes encoding a clostridial neurotoxin light chain and a second gene encoding a clostridial neurotoxin heavy chain, wherein the first and second genes are expressed in an oxidizing environment of the cell.

In a preferred embodiment, said cell is a prokaryote cell. In a more preferred embodiment, the oxidizing environment is the cytoplasm of the prokaryote cell.

In a preferred embodiment, the cell is a prokaryote cell in which at least one gene involved in disulphide bond formation is overexpressed by in the cytoplasm as compared to an otherwise identical wild-type cell and/or at least one gene involved in disulphide bond reduction is repressed as compared to an otherwise identical wild-type cell.

In one embodiment, the prokaryote cell is an E. coli cell from strain selected from AD494, BL21trxB, Origami, Rosetta-gami and SHuffle strains. In a preferred embodiment, the prokaryote cell is an E. coli cell from an Origami or Shuffle strain.

In one embodiment, the first gene encoding a clostridial neurotoxin light chain and the second gene encoding a clostridial neurotoxin heavy chain are present on the same vector.

In one embodiment, the first gene encoding a clostridial neurotoxin light chain and the second gene encoding a clostridial neurotoxin heavy chain are present on different vectors.

In a further aspect, the present invention provides a kit comprising
a. a cell comprising an oxidizing environment,
b. a first gene encoding a clostridial neurotoxin light chain, and
c. a second gene encoding a clostridial neurotoxin heavy chain,
wherein said first and second genes are suitable for separately expressing a clostridial neurotoxin light and a heavy chain in said oxidizing environment of said cell.

In a further aspect, the present invention provides a di-chain clostridial neurotoxin obtained by the method according to the invention.

In a further aspect, the present invention provides a pharmaceutical composition comprising a di-chain clostridial neurotoxin according to the invention. Preferably, the pharmaceutical composition comprises a di-chain clostridial neurotoxin according to the invention together with at least one component selected from a pharmaceutically acceptable carrier, excipient, adjuvant, propellant and/or salt.

In another aspect, the invention provides a di-chain clostridial neurotoxin according to the invention or pharmaceutical composition according to the invention for use in therapy.

In another aspect, the invention provides a method of treatment comprising the administration of a suitable dose of a di-chain clostridial neurotoxin according to the invention or pharmaceutical composition according to the invention to a patient in need thereof.

A di-chain clostridial neurotoxin according to the invention is suitable for use in treating a condition associated with unwanted neuronal activity, for example a condition selected from the group consisting of spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, tics, tremors, bruxism, anal fissure, achalasia, dysphagia and other muscle tone disorders and other disorders characterized by involuntary movements of muscle groups, lacrimation, hyperhidrosis, excessive salivation, excessive gastrointestinal secretions, secretory disorders, pain from muscle spasms, headache pain, migraine and dermatological conditions.

In another aspect, the invention provides a non-therapeutic use of a di-chain clostridial neurotoxin according to the invention for treating an aesthetic or cosmetic condition.

The di-chain clostridial neurotoxin according to the invention may be formulated for oral, parenteral, continuous infusion, inhalation or topical application. Compositions suitable for injection may be in the form of solutions, suspensions or emulsions, or dry powders which are dissolved or suspended in a suitable vehicle prior to use.

In the case of a di-chain clostridial neurotoxin according to the invention that is to be delivered locally, the chimeric neurotoxin may be formulated as a cream (e.g. for topical application), or for sub-dermal injection.

Local delivery means may include an aerosol, or other spray (e.g. a nebuliser). In this regard, an aerosol formulation of a chimeric neurotoxin enables delivery to the lungs and/or other nasal and/or bronchial or airway passages.

Di-chain clostridial neurotoxins according to the invention may be administered to a patient by intrathecal or epidural injection in the spinal column at the level of the spinal segment involved in the innervation of an affected organ.

A preferred route of administration is via laparoscopic and/or localised, particularly intramuscular, injection.

The dosage ranges for administration of the di-chain clostridial neurotoxins according to the invention are those to produce the desired therapeutic effect. It will be appreciated that the dosage range required depends on the precise nature of the di-chain clostridial neurotoxin or composition, the route of administration, the nature of the formulation, the age of the patient, the nature, extent or severity of the patient's condition, contraindications, if any, and the judgement of the attending physician. Variations in these dosage levels can be adjusted using standard empirical routines for optimisation.

Fluid dosage forms are typically prepared utilising the di-chain clostridial neurotoxin according to the invention and a pyrogen-free sterile vehicle. The di-chain clostridial neurotoxin, depending on the vehicle and concentration used, can be either dissolved or suspended in the vehicle. In preparing solutions the di-chain clostridial neurotoxin can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers may be sterilised by autoclaving. Advantageously additives such as buffering, solubilising, stabilising, preservative or bactericidal, suspending or emulsifying agents and or local anaesthetic agents may be dissolved in the vehicle.

Dry powders, which are dissolved or suspended in a suitable vehicle prior to use, may be prepared by filling pre-sterilised ingredients into a sterile container using aseptic technique in a sterile area. Alternatively the ingredients may be dissolved into suitable containers using aseptic technique in a sterile area. The product is then freeze dried and the containers are sealed aseptically.

Parenteral suspensions, suitable for intramuscular, subcutaneous or intradermal injection, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

Administration in accordance with the present invention may take advantage of a variety of delivery technologies including microparticle encapsulation, viral delivery systems or high-pressure aerosol impingement.

In a further aspect, the invention provides the use of a host cell which has an oxidative cytoplasm for producing a di-chain clostridial neurotoxin, wherein the host cell comprises a first gene encoding a clostridial neurotoxin light chain and a second gene encoding a clostridial neurotoxin heavy chain, wherein the first and second genes are expressed in the cytoplasm of the host cell.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a clostridial neurotoxin” includes a plurality of such candidate agents and reference to “the clostridial neurotoxin” includes reference to one or more clostridial neurotoxins and equivalents thereof known to those skilled in the art, and so forth.

The invention will now be described, by way of example only, with reference to the following Figures and Examples.

### FIGURE LEGENDS

FIG. 1—Western blot with a polyclonal in-house antibodies raised against either the LC of BoNT/A (FIG. 1A) or full length BoNT/A1—preference towards HC (FIG. 1B).MK: Magic Mark, Sample 1: Control, Shuffle T7 lysate—No IPTG, Sample 2: Origami 2 lysate±DTT, Sample 3: Shuffle T7 lysate±DTT, Sample 4: Shuffle T7 Express lysate±DTT, Sample 5: BL21 (DE3)±DTT.

FIG. 2—SDS PAGE following purification of Co-expressed BoNT/A1(0) and Single-chain expressed BoNT/A1(0). MK: Bench Mark, Sample 1: Co-expressed BoNT/A1(0), 2: Single-chain expressed BoNT/A1(0), Sample 3: Co-expressed BoNT/A1(0) reduced, Sample 4: Single-chain expressed BoNT/A1(0) reduced.

FIG. 3—Optim read out. In all figures, lane 1 is purified single-chain expressed BoNT/A1(0) and Lane 2 is purified co-expressed BoNT/A1(0). FIG. 3A) is a measure of temperature dependent shift in fluorescence emission barycentric mean (BCM). FIG. 3B) is a measure of temperature dependent shift in static light scatter (SLS) at 266 nm and FIG. 3C) is a measure of temperature dependent shift in (SLS) at 473 nm. Average and standard deviation are shown from 4 replicate reads for each molecule.

FIG. 4—SEC read out at 280 nm, FIG. 4 shows the full scale chromatogram at 280 nm. Purified co-expressed BoNT/A1(0) and purified single-chain expressed BoNT/A1(0) have been annotated.

FIG. 5—Glutamate release assay. The Figure compares co-expressed BoNT/A1 (SXN104279-DK170710) against native clostridial BoNT/A1 (LIST Biological Laboratories) on their ability to inhibit glutamate release in rat cerebral cortical neurones.

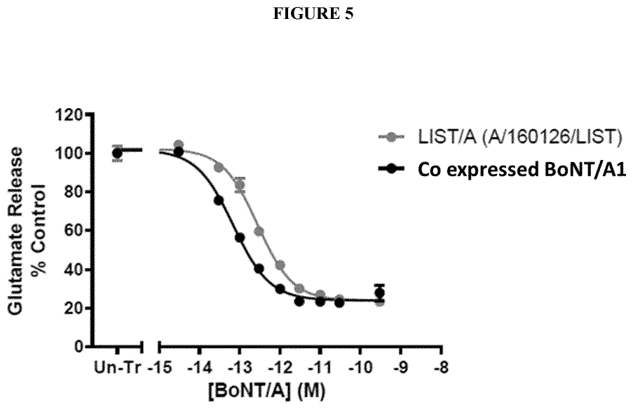

### EXAMPLES

### Example 1—Co-Expression of BoNT/A1(0) Light and Heavy Chains Using the Single Vector (Dual Promoter) Approach

Primers were designed to amplify separately the light chain (Table 3—Primers 1 and 2) and the heavy chain (Table 3—Primers 3 and 4) of endonegative BoNT/A1(0) ensuring that a stop codon would be incorporated at the end of the Light chain (LC) and a start codon at the beginning of the Heavy chain (HC). Also included were the restriction sites NcoI (fwrd) and BamHI (rev) to allow the LC to be ligated into MSC 1 of the pETDuet vector (Millipore #71146) while NdeI (fwrd) and XhoI (rev) were used to be able to ligate the HC into MSC 2. Genes were amplified with Q5 Hot start HF master mix (NEB #M0494S) using BoNT/A1(0) template DNA shown in Table 4. The amplified LC and pETDuet vector were then digested with NcoI (NEB #R3193) and BamHI (NEB #R3136) and ligated using NEB T4 DNA Ligase (#M02025).

TABLE 3
Primers used to insert BoNT/A1(0) LC into
MSC 1 and HC into MSC 2 of PetDuet

| Primers | Sequence (5′->3′) |
|---|---|
| 1) LC-A1 forward | ATACACCATGGTATGCCATTCGTCAACAA |
| (Nco1) | GCAATT (SEQ ID NO: 20) |
| 2) LC-A1 reverse | GCTTTTGGATCCGGTTTATTTGCTGGTGA |
| (BamH1) | TGATACCGCGC (SEQ ID NO: 21) |
| 3) HC-A1 forward | ACAAGCATATGGCGCTGAATGACCTGTGC |
| (Nde1) | ATTAAG (SEQ ID NO: 22) |
| 4) HC-A1 reverse | AAGCTTCTCGAGTCATTACAGCGGACGTT |
| (Xho1) | CGCCCC (SEQ ID NO: 23) |

TABLE 4
LC, activation loop and HC Sequences for BoNT/A1(0)

| | |
|---|---|
| Nucleic acid sequence of BoNT/A1(0) LC | SEQ ID NO: 17 |
| Activation loop | SEQ ID NO: 18 |
| Nucleic acid sequence of BoNT/A1(0) HC | SEQ ID NO: 19 |

Next, the resulting pETDuet/LC vector and the amplified HC gene were digested with Xho1 (NEB #R0146S) and Nde (NEB #R0111S) and ligated together resulting in the desired final construct.

To test co-expression of BoNT/A1(0) the vector was transformed into Shuffle T7 ((NEB #C3026H), Shuffle T7 Express cells (NEB #C3029), BL21(DE3) (C25271) and Origami 2 cells (Merks #714083) as instructed and the resulting colonies were stored as microbank beads at −80° C. Note all cloning and transformation steps followed manufacturer's instructions.

For the expression, 100 ml of modified TB (mTB) (Melford #T1703) containing 50 μg/ml Ampicillin in 250 ml baffled flasks were set up for each of the overnight cultures. These were inoculated with one microbank bead for each of the cell lines and grown overnight at 30° C. for 20 hours while shaking at 225 rpm. The next day the main cultures were set up using 900 ml of mTB+50 μg/ml Ampicillin in 2.5 L baffled flasks which were inoculated with 10 ml of the overnight culture. Cell density was allowed to reach an OD600 of 1 by growing at 30° C. while shaking at 225 rpm. Once the desired OD was reached the temperature was allowed to drop to 16° C. (1 hour) before inducing with 1 mM IPTG (Sigma #I6758). Expression cultures were incubated at 16° C. for a further 20 hours prior to recovering cells at 6000 rpm for 30 minutes.

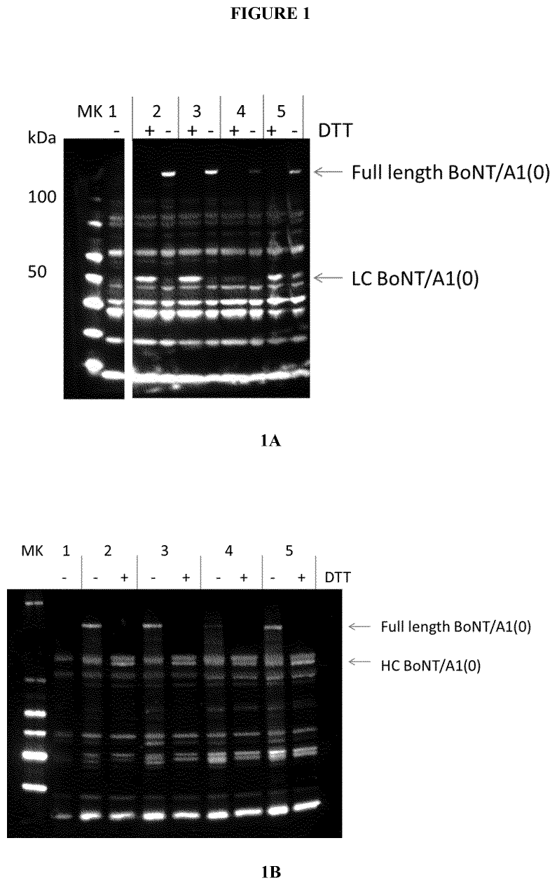

Recovered cells from the expressions were re-suspended with 6 ml/g using 25 mM Tris, 150 mM NaCl pH 8 and then soluble protein was extracted by one pass through a constant systems homogenisior at 20 Kpsi. Cell debris was removed by centrifugation at 12 000 rpm for 30 minutes and then the clarified lysate was assessed by Western blot (FIG. 1).

Briefly, clarified lysates were diluted 1:10 with either ThermoFishers NuPAGE® LDS Sample Buffer (4×) #NP0007+0.1 M DTT (Sigma) for the reduced samples or Novex® Tris-Glycine SDS Sample Buffer (2×) #LC2676 for the non-reduced samples. Following heating at 95° C. for 10 minutes, SDS PAGE electrophoresis was performed on these samples using 4-12% Bis Tris acrylamide gels. Proteins were transferred to 0.2 μM nitrocellulose membranes prior to blotting with polyclonal in-house antibodies raised against either the LC of BoNT/A1 or full length BoNT/A1—preference towards HC. Antibody binding was detected using an Anti-Rabbit IgG—Peroxidase antibody (Sigma #A0545-1ML) and visualized using Super Signal West Dura extended duration substrate.

The results presented in FIG. 1 show that a band of 150 kDa equating to full length BoNT/A1(0) is present in Samples 2, 3 and 5 (also sample 4 to a lesser degree) but is not seen in the negative control. The fact that the 150 kDa protein is no longer visible in the presence of DTT confirms this as the disulphide bond which has been reduced and now the LC can be seen at 50 kDa in FIG. 1A and the HC at 100 kDa in FIG. 1B.

These results confirm that intracellular formation of the BoNT/A1(0) disulphide bridge following co-expression of the light and heavy chains is feasible in all the strains and that minimal amounts of free LC are present when using expression strains containing an oxidative cytoplasm as compared when a strain with a reducing cytoplasm is used (BL21 (DE3)).

### Example 2—Purification of BoNT/A1(0) Following Co-Expression of the Light and Heavy Chains in Shuffle T7 Cells

3 Litres of BoNT/A1(0) culture were again co-expressed in Shuffle T7 cells and lysed as detailed in example 1. The resultant full length BoNT/A1(0) was purified from clarified lysate using 3 chromatography steps as follows:

### Step 1: Butyl HP

The clarified lysate was diluted in half by the addition of 25 mM Tris, 2 M (NH₄)₂SO₄ pH 8 to bring the (NH₄)₂SO₄ concentration up to 1 M. The sample was then loaded onto a pre equilibrated 10 ml Butyl HP column (2×5 ml HiTrap Butyl HP, GE Healthcare #28-4110-05) at 150 cm/hr. Following a 10 column volume (CV) wash using 25 mM Tris, 1 M (NH₄)₂SO₄ pH 8, any bound proteins were eluted over a 25 CV linear gradient down to 25 mM Tris, 35 mM NaCl pH 8 collecting 5 ml fractions. Fractions were then analysed by SDS PAGE and those that contained the target toxin were pooled.

### Step 2: Q HP

The Butyl HP pool was buffer exchanged into a low salt buffer so that it could be loaded onto a Q HP column. This was achieved by performing several runs of buffer exchange into 25 mM Tris, 20 mM NaCl pH 8 using a HiPrep26/10 desalting column (GE healthcare, #17-5087-01) and following manufacturer's instructions.

The sample was then loaded onto a pre equilibrated 4.7 ml HiScreen Q HP column (GE healthcare, #28-9505-11) at 75 cm/hr. Following a 5 CV wash with 25 mM Tris, 20 mM NaCl pH 8, bound proteins were eluted over a 25 CV linear gradient up to 25 mM Tris, 300 mM NaCl pH 8 collecting 2.5 ml fractions. Following analysis by SDS PAGE, the fractions containing target protein were pooled.

### Step 3: Phenyl HP

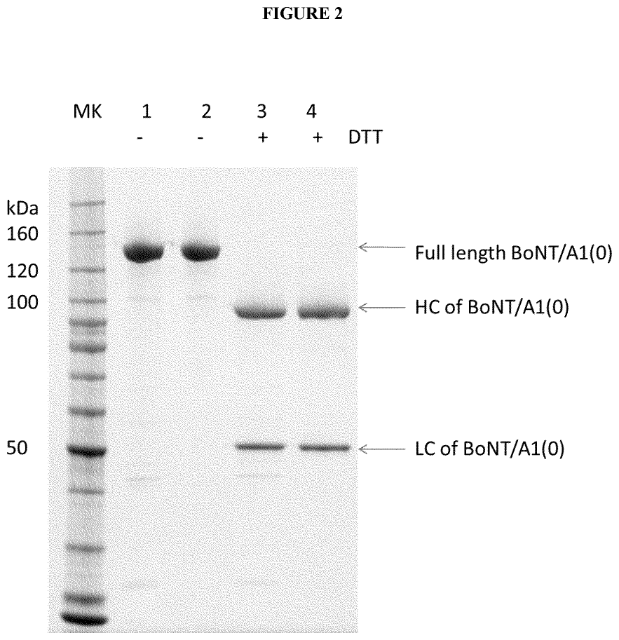

The Q HP pool was conditioned for the Phenyl HP column by diluting the sample in half with 25 mM Tris, 2 M (NH₄)₂SO₄ pH 8 to bring the (NH₄)₂SO₄ up to 1 M. The sample was loaded onto a pre equilibrated 1 ml Phenyl HP (GE Healthcare #17-1351-01) column at 150 cm/hr and then the column was washed with 3 CV of 25 mM Tris, 1 M (NH₄)₂SO₄ pH 8. Elution of bound proteins used a 25 CV linear gradient down to 25 mM Tris, 35 mM NaClpH 8 collecting 0.5 ml fractions. Following analysis by SDS PAGE, fractions containing the target protein were pooled resulting in the final product as shown in FIG. 2.

To be used as a control, single chain recombinant BoNT/A1(0) was also expressed and purified. To achieve this, BoNT/A1(0) (Table 4—LC+Activation loop+HC) was inserted into pJ401 so that it could be expressed as a single chain product using the BLR (DE3) E. coli expression strain (Novagen #69053).

For the expression, 100 ml of modified TB (mTB) (Melford #T1703) containing 30 μg/ml Kanamycin in 250 ml baffled flasks was set up for the overnight culture. This was inoculated with one microbank bead grown overnight at 37° C. for 20 hours shaking at 225 rpm. The next day the main cultures were set up using 15×1 L of mTB+30 μg/ml Kanamycin in 2.5 L baffled flasks which were each inoculated with 10 ml of the overnight culture. Cell density was allowed to reach an OD600 of 0.5 by growing at 37° C. while shaking at 225 rpm. Once the desired OD was reached the temperature was allowed to drop to 16° C. (1 hour) before inducing with 1 mM IPTG (Sigma #I6758). Expression cultures were incubated at 16° C. for a further 20 hours prior to recovering cells at 5000 rpm for 20 minutes.

Recovered cells were lysed and toxin purified as with the Co-expressed BoNT/A1(0). The only 2 differences were that the purification was performed at a larger scale and also required an activation step between the 2^{nd} and 3^{rd} columns:

200 ml Butyl HP->53 ml Q HP->Activation (See below)->10 ml Phenyl HP

Activation stage—The Q HP pool (0.46 mg/ml by A280) was incubated with 92 μg (0.8 μg Lys-C/ml of sample) of Lys-C(Sigma #P2289) at 4° C. for 20 hours. Following activation the sample was immediately diluted in half with 25 mM Tris pH 8, 2 M (NH₄)₂SO₄ so that it could be loaded onto the Phenyl HP, purification was then continued as with Example 1.

The two final products resulting from the Phenyl HP column were assessed by SDS-PAGE (FIG. 2). Briefly, the Phenyl HP pool containing Co-expressed and single-chain expressed BoNT/A1(0) was diluted to 0.1 mg/ml with either ThermoFishers NuPAGE® LDS Sample Buffer (4×) #NP0007+0.1 M DTT (Sigma) for the reduced samples or Novex® Tris-Glycine SDS Sample Buffer (2×) #LC2676 for the non-reduced samples. Single-chain expressed BoNT/A1(0) purified using the same process including the additional activation stage was used as the control. Prepared SDS samples were heated at 95° C. for 10 minutes, SDS PAGE electrophoresis was performed on these samples using 4-12% Bis Tris acrylamide gels. Staining used SimplyBlue SafeStain (Thermo fisher #LC6065) for one hour and then destained overnight.

The results shown in FIG. 2 confirm that purification was successful with co-expressed BoNT/A1(0) behaving in the same manner as single-chain expressed BoNT/A1(0) suggesting correct folding.

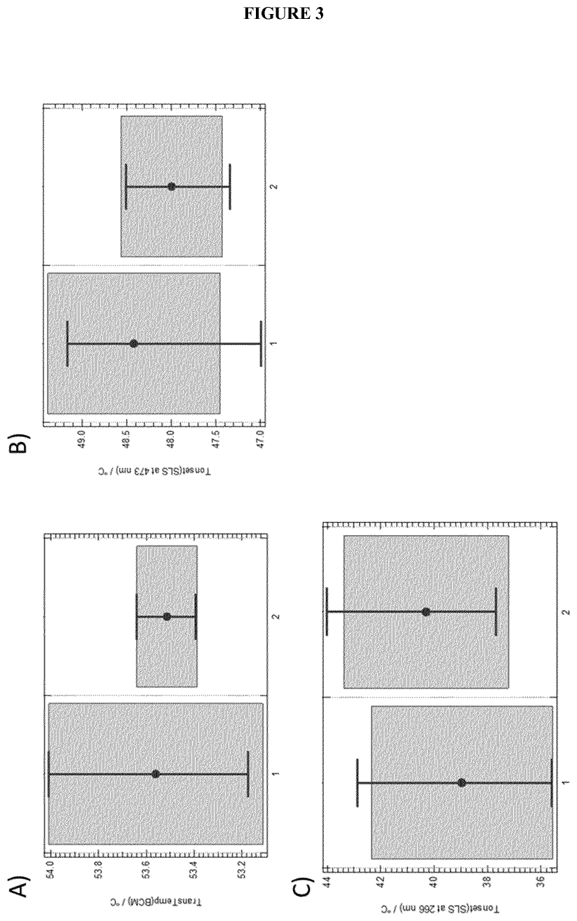

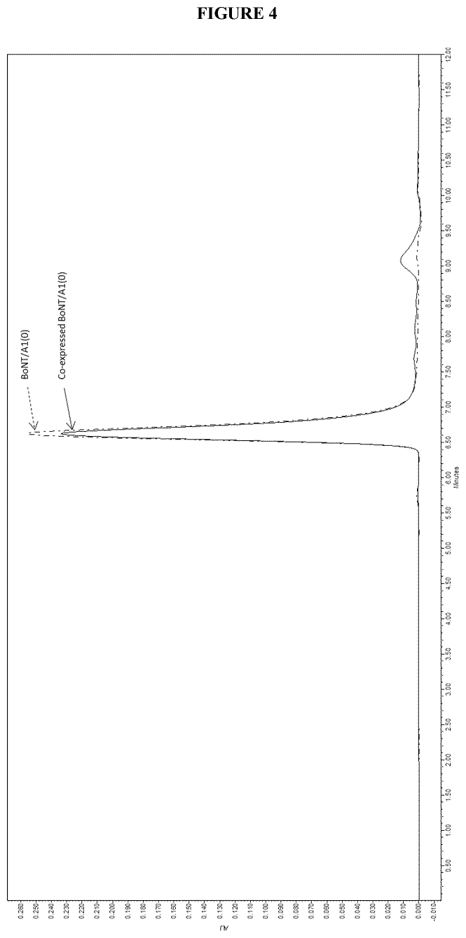

The purified samples were also compared on the optim which is a device that measures Intrinsic florescence and Light scattering giving an indication on folding and stability (FIG. 3) and Size exclusion chromatography (SEC) for size and aggregation profile (FIG. 4).

The Optim results show that Co-expressed BoNT A1(0) and single-chain expressed BoNT/A1(0) have very similar transition points in BCM, SLS at 266 and 473 nm which are readouts for melting temperature and small and large particle aggregation respectively.

The SEC results shows that Co-expressed BoNT A1(0) and single-chain expressed BoNT/A1(0) have identical monomer peaks with minimal aggregation in both.

### Example 3—Co-Expression of BoNT/A1 and Glutamate Release Assay to Confirm Activity

Primers were designed to mutate two residues (Q224E/Y227H) within the LC domain (SEQ ID NO 17) of the BoNT/A1(0) pETDUET co-expression vector, in order to restore zinc-binding essential to the proteolytic activity of this domain. The resulting pETDUET vector will co-express active BoNT/A1 LC and HC, therefore allowing confirmation of potency in a cell-based system. The mutations were introduced using quick change lightning mutagenesis (#210514—Agilent technologies) following manufacturer's instructions.

The resulting vector was transformed into Shuffle T7 cells and expression/purification were performed as described in Example 1—co-expression of BoNT/A1(0) and Example 2—purification of co-expressed BoNT/A1(0). Note, that this molecule only required the first two chromatography columns, Butyl HP and Q HP as it does not require an activation step.

Co-expressed full length BoNT/A1 was then tested on the Rat Ctx Glutamate Release assay which will confirm translocation and snare cleavage by inhibition of glutamate release as a result of BoNT activity. Commercial native BoNT/A1 (LIST biological laboratories) was used as a control against the co-expressed BoNT/A1.

The glutamate release assay showed that co-expressed BoNT/A1 inhibits glutamate release with potency comparable to that of the native BoNT/A. This therefore demonstrates that co-expression is a viable method for production of fully active di-chain clostridial neurotoxin capable of performing all required steps for intoxication (binding and internalisation at the neuronal endplate, translocation of the light chain from the endosome into the cytoplasm and proteolytic cleavage of the target SNARE protein).

| SEQUENCES | |
|---|---|
| BoNT/A1, accession number A5HZZ9, amino acid sequence. | |
| SEQ ID NO: 1 | |
| MPFVNKQFNYKDPVNGVDIAYIKIPNAGQMQPVKAFKIHNKIWVIPERDTFTNP | |
| EEGDLNPPPEAKQVPVSYYDSTYLSTDNEKDNYLKGVTKLFERIYSTDLGRMLL | |
| TSIVRGIPFWGGSTIDTELKVIDTNCINVIQPDGSYRSEELNLVIIGPSADIIQFECK | |
| SFGHEVLNLTRNGYGSTQYIRFSPDFTFGFEESLEVDTNPLLGAGKFATDPAVTL | |
| AHELIHAGHRLYGIAINPNRVFKVNTNAYYEMSGLEVSFEELRTFGGHDAKFID | |
| SLQENEFRLYYYNKFKDIASTLNKAKSIVGTTASLQYMKNVFKEKYLLSEDTSG | |
| KFSVDKLKFDKLYKMLTEIYTEDNFVKFFKVLNRKTYLNFDKAVFKINIVPKVN | |
| YTIYDGFNLRNTNLAANFNGQNTEINNMNFTKLKNFTGLFEFYKLLCVRGIITS | |
| KTKSLDKGYNKALNDLCIKVNNWDLFFSPSEDNFTNDLNKGEEITSDTNIEAAE | |
| ENISLDLIQQYYLTFNFDNEPENISIENLSSDIIGQLELMPNIERFPNGKKYELDKY | |
| TMFHYLRAQEFEHGKSRIALTNSVNEALLNPSRVYTFFSSDYVKKVNKATEAA | |
| MFLGWVEQLVYDFTDETSEVSTTDKIADITIIIPYIGPALNIGNMLYKDDFVGALI | |
| FSGAVILLEFIPEIAIPVLGTFALVSYIANKVLTVQTIDNALSKRNEKWDEVYKYI | |
| VTNWLAKVNTQIDLIRKKMKEALENQAEATKAIINYQYNQYTEEEKNNINFNID | |
| DLSSKLNESINKAMININKFLNQCSVSYLMNSMIPYGVKRLEDFDASLKDALLK | |
| YIYDNRGTLIGQVDRLKDKVNNTLSTDIPFQLSKYVDNQRLLSTFTEYIKNIINTS | |
| ILNLRYESNHLIDLSRYASKINIGSKVNFDPIDKNQIQLFNLESSKIEVILKNAIVY | |
| NSMYENFSTSFWIRIPKYFNSISLNNEYTIINCMENNSGWKVSLNYGEIIWTLQD | |
| TQEIKQRVVFKYSQMINISDYINRWIFVTITNNRLNNSKIYINGRLIDQKPISNLG | |
| NIHASNNIMFKLDGCRDTHRYIWIKYFNLFDKELNEKEIKDLYDNQSNSGILKD | |
| FWGDYLQYDKPYYMLNLYDPNKYVDVNNVGIRGYMYLKGPRGSVMTTNIYL | |
| NSSLYRGTKFIIKKYASGNKDNIVRNNDRVYINVVVKNKEYRLATNASQAGVE | |
| KILSALEIPDVGNLSQVVVMKSKNDQGITNKCKMNLQDNNGNDIGFIGFHQFN | |
| NIAKLVASNWYNRQIERSSRTLGCSWEFIPVDDGWGERPL. | |
| BoNT/B1, accession number B1INP5, amino acid sequence. | |
| SEQ ID NO: 2 | |
| MPVTINNFNYNDPIDNNNIIMMEPPFARGTGRYYKAFKITDRIWIIPERYTFGYK | |
| PEDFNKSSGIFNRDVCEYYDPDYLNTNDKKNIFLQTMIKLFNRIKSKPLGEKLLE | |
| MIINGIPYLGDRRVPLEEFNTNIASVTVNKLISNPGEVERKKGIFANLIIFGPGPVL | |
| NENETIDIGIQNHFASREGFGGIMQMKFCPEYVSVFNNVQENKGASIFNRRGYFS | |
| DPALILMHELIHVLHGLYGIKVDDLPIVPNEKKFFMQSTDAIQAEELYTFGGQDP | |
| SIITPSTDKSIYDKVLQNFRGIVDRLNKVLVCISDPNININIYKNKFKDKYKFVED | |
| SEGKYSIDVESFDKLYKSLMFGFTETNIAENYKIKTRASYFSDSLPPVKIKNLLD | |
| NEIYTIEEGFNISDKDMEKEYRGQNKAINKQAYEEISKEHLAVYKIQMCKSVKA | |
| PGICIDVDNEDLFFIADKNSFSDDLSKNERIEYNTQSNYIENDFPINELILDTDLIS | |
| KIELPSENTESLTDFNVDVPVYEKQPAIKKIFTDENTIFQYLYSQTFPLDIRDISLT | |
| SSFDDALLFSNKVYSFFSMDYIKTANKVVEAGLFAGWVKQIVNDFVIEANKSN | |
| TMDKIADISLIVPYIGLALNVGNETAKGNFENAFEIAGASILLEFIPELLIPVVGAF | |
| LLESYIDNKNKIIKTIDNALTKRNEKWSDMYGLIVAQWLSTVNTQFYTIKEGMY | |
| KALNYQAQALEEIIKYRYNIYSEKEKSNINIDFNDINSKLNEGINQAIDNINNFIN | |
| GCSVSYLMKKMIPLAVEKLLDFDNTLKKNLLNYIDENKLYLIGSAEYEKSKVN | |
| KYLKTIMPFDLSIYTNDTILIEMFNKYNSEILNNIILNLRYKDNNLIDLSGYGAKV | |
| EVYDGVELNDKNQFKLTSSANSKIRVTQNQNIIFNSVFLDFSVSFWIRIPKYKND | |
| GIQNYIHNEYTIINCMKNNSGWKISIRGNRIIWTLIDINGKTKSVFFEYNIREDISE | |
| YINRWFFVTITNNLNNAKIYINGKLESNTDIKDIREVIANGEIIFKLDGDIDRTQFI | |
| WMKYFSIFNTELSQSNIEERYKIQSYSEYLKDFWGNPLMYNKEYYMFNAGNKN | |
| SYIKLKKDSPVGEILTRSKYNQNSKYINYRDLYIGEKFIIRRKSNSQSINDDIVRK | |
| EDYIYLDFFNLNQEWRVYTYKYFKKEEEKLFLAPISDSDEFYNTIQIKEYDEQPT | |
| YSCQLLFKKDEESTDEIGLIGIHRFYESGIVFEEYKDYFCISKWYLKEVKRKPYN | |
| LKLGCNWQFIPKDEGWTE. | |
| BoNT/C1, accession number P18640, amino acid sequence. | |
| SEQ ID NO: 3 | |
| MPITINNFNYSDPVDNKNILYLDTHLNTLANEPEKAFRITGNIWVIPDRFSRNSNP | |
| NLNKPPRVTSPKSGYYDPNYLSTDSDKDPFLKEIIKLFKRINSREIGEELIYRLSTD | |
| IPFPGNNNTPINTFDFDVDFNSVDVKTRQGNNWVKTGSINPSVIITGPRENIIDPE | |
| TSTFKLTNNTFAAQEGFGALSIISISPRFMLTYSNATNDVGEGRFSKSEFCMDPIL | |
| ILMHELNHAMHNLYGIAIPNDQTISSVTSNIFYSQYNVKLEYAEIYAFGGPTIDLI | |
| PKSARKYFEEKALDYYRSIAKRLNSITTANPSSFNKYIGEYKQKLIRKYRFVVES | |
| SGEVTVNRNKFVELYNELTQIFTEFNYAKIYNVQNRKIYLSNVYTPVTANILDD | |
| NVYDIQNGENIPKSNLNVLFMGQNLSRNPALRKVNPENMLYLFTKFCHKAIDG | |
| RSLYNKTLDCRELLVKNTDLPFIGDISDVKTDIFLRKDINEETEVIYYPDNVSVD | |
| QVILSKNTSEHGQLDLLYPSIDSESEILPGENQVFYDNRTQNVDYLNSYYYLESQ | |
| KLSDNVEDFTFTRSIEEALDNSAKVYTYFPTLANKVNAGVQGGLFLMWANDV | |
| VEDFTTNILRKDTLDKISDVSAIIPYIGPALNISNSVRRGNFTEAFAVTGVTILLEA | |
| FPEFTIPALGAFVIYSKVQERNEIIKTIDNCLEQRIKRWKDSYEWMMGTWLSRIIT | |
| QFNNISYQMYDSLNYQAGAIKAKIDLEYKKYSGSDKENIKSQVENLKNSLDVKI | |
| SEAMNNINKFIRECSVTYLFKNMLPKVIDELNEFDRNTKAKLINLIDSHNIILVGE | |
| VDKLKAKVNNSFQNTIPFNIFSYTNNSLLKDIINEYFNNINDSKILSLQNRKNTLV | |
| DTSGYNAEVSEEGDVQLNPIFPFDFKLGSSGEDRGKVIVTQNENIVYNSMYESFS | |
| ISFWIRINKWVSNLPGYTIIDSVKNNSGWSIGIISNFLVFTLKQNEDSEQSINFSYD | |
| ISNNAPGYNKWFFVTVTNNMMGNMKIYINGKLIDTIKVKELTGINFSKTITFEIN | |
| KIPDTGLITSDSDNINMWIRDFYIFAKELDGKDINILFNSLQYTNVVKDYWGNDL | |
| RYNKEYYMVNIDYLNRYMYANSRQIVFNTRRNNNDFNEGYKIIIKRIRGNTND | |
| TRVRGGDILYFDMTINNKAYNLFMKNETMYADNHSTEDIYAIGLREQTKDIND | |
| NIIFQIQPMNNTYYYASQIFKSNFNGENISGICSIGTYRFRLGGDWYRHNYLVPT | |
| VKQGNYASLLESTSTHWGFVPVSE. | |
| BoNT/D, accession number P19321, amino acid sequence. | |
| SEQ ID NO: 4 | |
| MTWPVKDFNYSDPVNDNDILYLRIPQNKLITTPVKAFMITQNIWVIPERFSSDTN | |
| PSLSKPPRPTSKYQSYYDPSYLSTDEQKDTFLKGIIKLFKRINERDIGKKLINYLV | |
| VGSPFMGDSSTPEDTFDFTRHTTNIAVEKFENGSWKVTNIITPSVLIFGPLPNILD | |
| YTASLTLQGQQSNPSFEGFGTLSILKVAPEFLLTFSDVTSNQSSAVLGKSIFCMDP | |
| VIALMHELTHSLHQLYGINIPSDKRIRPQVSEGFFSQDGPNVQFEELYTFGGLDV | |
| EIIPQIERSQLREKALGHYKDIAKRLNNINKTIPSSWISNIDKYKKIFSEKYNFDKD | |
| NTGNEVVNIDKENSLYSDLTNVMSEVVYSSQYNVKNRTHYFSRHYLPVFANIL | |
| DDNIYTIRDGFNLTNKGFNIENSGQNIERNPALQKLSSESVVDLFTKVCLRLTKN | |
| SRDDSTCIKVKNNRLPYVADKDSISQEIFENKIITDETNVQNYSDKFSLDESILDG | |
| QVPINPEIVDPLLPNVNMEPLNLPGEEIVFYDDITKYVDYLNSYYYLESQKLSNN | |
| VENITLTTSVEEALGYSNKIYTFLPSLAEKVNKGVQAGLFLNWANEVVEDFTTN | |
| IMKKDTLDKISDVSVIIPYIGPALNIGNSALRGNFNQAFATAGVAFLLEGFPEFTI | |
| PALGVFTFYSSIQEREKIIKTIENCLEQRVKRWKDSYQWMVSNWLSRITTQFNHI | |
| NYQMYDSLSYQADAIKAKIDLEYKKYSGSDKENIKSQVENLKNSLDVKISEAM | |
| NNINKFIRECSVTYLFKNMLPKVIDELNKFDLRTKTELINLIDSHNIILVGEVDRL | |
| KAKVNESFENTMPFNIFSYTNNSLLKDIINEYFNSINDSKILSLQNKKNALVDTS | |
| GYNAEVRVGDNVQLNTIYTNDFKLSSSGDKIIVNLNNNILYSAIYENSSVSFWIK | |
| ISKDLTNSHNEYTIINSIEQNSGWKLCIRNGNIEWILQDVNRKYKSLIFDYSESLS | |
| HTGYTNKWFFVTITNNIMGYMKLYINGELKQSQKIEDLDEVKLDKTIVFGIDEN | |
| IDENQMLWIRDFNIFSKELSNEDINIVYEGQILRNVIKDYWGNPLKFDTEYYIIND | |
| NYIDRYIAPESNVLVLVQYPDRSKLYTGNPITIKSVSDKNPYSRILNGDNIILHML | |
| YNSRKYMIIRDTDTIYATQGGECSQNCVYALKLQSNLGNYGIGIFSIKNIVSKNK | |
| YCSQIFSSFRENTMLLADIYKPWRFSFKNAYTPVAVTNYETKLLSTSSFWKFISR | |
| DPGWVE. | |
| BoNT/E1, accession number WP_003372387, amino acid sequence. | |
| SEQ ID NO: 5 | |
| MPKINSFNYNDPVNDRTILYIKPGGCQEFYKSFNIMKNIWIIPERNVIGTTPQDFH | |
| PPTSLKNGDSSYYDPNYLQSDEEKDRFLKIVTKIFNRINNNLSGGILLEELSKANP | |
| YLGNDNTPDNQFHIGDASAVEIKFSNGSQDILLPNVIIMGAEPDLFETNSSNISLR | |
| NNYMPSNHGFGSIAIVTFSPEYSFRFNDNSMNEFIQDPALTLMHELIHSLHGLYG | |
| AKGITTKYTITQKQNPLITNIRGTNIEEFLTFGGTDLNIITSAQSNDIYTNLLADYK | |
| KIASKLSKVQVSNPLLNPYKDVFEAKYGLDKDASGIYSVNINKFNDIFKKLYSFT | |
| EFDLATKFQVKCRQTYIGQYKYFKLSNLLNDSIYNISEGYNINNLKVNFRGQNA | |
| NLNPRIITPITGRGLVKKIIRFCKNIVSVKGIRKSICIEINNGELFFVASENSYNDDN | |
| INTPKEIDDTVTSNNNYENDLDQVILNFNSESAPGLSDEKLNLTIQNDAYIPKYD | |
| SNGTSDIEQHDVNELNVFFYLDAQKVPEGENNVNLTSSIDTALLEQPKIYTFFSS | |
| EFINNVNKPVQAALFVSWIQQVLVDFTTEANQKSTVDKIADISIVVPYIGLALNI | |
| GNEAQKGNFKDALELLGAGILLEFEPELLIPTILVFTIKSFLGSSDNKNKVIKAIN | |
| NALKERDEKWKEVYSFIVSNWMTKINTQFNKRKEQMYQALQNQVNAIKTIIES | |
| KYNSYTLEEKNELTNKYDIKQIENELNQKVSIAMNNIDRFLTESSISYLMKLINE | |
| VKINKLREYDENVKTYLLNYIIQHGSILGESQQELNSMVTDTLNNSIPFKLSSYT | |
| DDKILISYFNKFFKRIKSSSVLNMRYKNDKYVDTSGYDSNININGDVYKYPTNK | |
| NQFGIYNDKLSEVNISQNDYIIYDNKYKNFSISFWVRIPNYDNKIVNVNNEYTIIN | |
| CMRDNNSGWKVSLNHNEIIWTLQDNAGINQKLAFNYGNANGISDYINKWIFVT | |
| ITNDRLGDSKLYINGNLIDQKSILNLGNIHVSDNILFKIVNCSYTRYIGIRYFNIFD | |
| KELDETEIQTLYSNEPNTNILKDFWGNYLLYDKEYYLLNVLKPNNFIDRRKDST | |
| LSINNIRSTILLANRLYSGIKVKIQRVNNSSTNDNLVRKNDQVYINFVASKTHLFP | |
| LYADTATTNKEKTIKISSSGNRFNQVVVMNSVGNNCTMNFKNNNGNNIGLLGF | |
| KADTVVASTWYYTHMRDHTNSNGCFWNFISEEHGWQEK. | |
| BoNT/F1, accession number Q57236, amino acid sequence. | |
| SEQ ID NO: 6 | |
| MPVVINSFNYNDPVNDDTILYMQIPYEEKSKKYYKAFEIMRNVWIIPERNTIGTD | |
| PSDFDPPASLENGSSAYYDPNYLTTDAEKDRYLKTTIKLFKRINSNPAGEVLLQE | |
| ISYAKPYLGNEHTPINEFHPVTRTTSVNIKSSTNVKSSIILNLLVLGAGPDIFENSS | |
| YPVRKLMDSGGVYDPSNDGFGSINIVTFSPEYEYTFNDISGGYNSSTESFIADPAI | |
| SLAHELIHALHGLYGARGVTYKETIKVKQAPLMIAEKPIRLEEFLTFGGQDLNII | |
| TSAMKEKIYNNLLANYEKIATRLSRVNSAPPEYDINEYKDYFQWKYGLDKNAD | |
| GSYTVNENKFNEIYKKLYSFTEIDLANKFKVKCRNTYFIKYGFLKVPNLLDDDI | |
| YTVSEGFNIGNLAVNNRGQNIKLNPKIIDSIPDKGLVEKIVKFCKSVIPRKGTKAP | |
| PRLCIRVNNRELFFVASESSYNENDINTPKEIDDTTNLNNNYRNNLDEVILDYNS | |
| ETIPQISNQTLNTLVQDDSYVPRYDSNGTSEIEEHNVVDLNVFFYLHAQKVPEG | |
| ETNISLTSSIDTALSEESQVYTFFSSEFINTINKPVHAALFISWINQVIRDFTTEATQ | |
| KSTFDKIADISLVVPYVGLALNIGNEVQKENFKEAFELLGAGILLEFVPELLIPTIL | |
| VFTIKSFIGSSENKNKIIKAINNSLMERETKWKEIYSWIVSNWLTRINTQFNKRKE | |
| QMYQALQNQVDAIKTVIEYKYNNYTSDERNRLESEYNINNIREELNKKVSLAM | |
| ENIERFITESSIFYLMKLINEAKVSKLREYDEGVKEYLLDYISEHRSILGNSVQEL | |
| NDLVTSTLNNSIPFELSSYTNDKILILYFNKLYKKIKDNSILDMRYENNKFIDISG | |
| YGSNISINGDVYIYSTNRNQFGIYSSKPSEVNIAQNNDIIYNGRYQNFSISFWVRIP | |
| KYFNKVNLNNEYTIIDCIRNNNSGWKISLNYNKIIWTLQDTAGNNQKLVFNYTQ | |
| MISISDYINKWIFVTITNNRLGNSRIYINGNLIDEKSISNLGDIHVSDNILFKIVGCN | |
| DTRYVGIRYFKVFDTELGKTEIETLYSDEPDPSILKDFWGNYLLYNKRYYLLNL | |
| LRTDKSITQNSNFLNINQQRGVYQKPNIFSNTRLYTGVEVIIRKNGSTDISNTDNF | |
| VRKNDLAYINVVDRDVEYRLYADISIAKPEKIIKLIRTSNSNNSLGQIIVMDSIGN | |
| NCTMNFQNNNGGNIGLLGFHSNNLVASSWYYNNIRKNTSSNGCFWSFISKEHG | |
| WQEN. | |
| BoNT/G, accession number WP_039635782, amino acid sequence. | |
| SEQ ID NO: 7 | |
| MPVNIKNFNYNDPINNDDIIMMEPFNDPGPGTYYKAFRIIDRIWIVPERFTYGFQ | |
| PDQFNASTGVFSKDVYEYYDPTYLKTDAEKDKFLKTMIKLFNRINSKPSGQRLL | |
| DMIVDAIPYLGNASTPPDKFAANVANVSINKKIIQPGAEDQIKGLMTNLIIFGPGP | |
| VLSDNFTDSMIMNGHSPISEGFGARMMIRFCPSCLNVFNNVQENKDTSIFSRRA | |
| YFADPALTLMHELIHVLHGLYGIKISNLPITPNTKEFFMQHSDPVQAEELYTFGG | |
| HDPSVISPSTDMNIYNKALQNFQDIANRLNIVSSAQGSGIDISLYKQIYKNKYDF | |
| VEDPNGKYSVDKDKFDKLYKALMFGFTETNLAGEYGIKTRYSYFSEYLPPIKTE | |
| KLLDNTIYTQNEGFNIASKNLKTEFNGQNKAVNKEAYEEISLEHLVIYRIAMCK | |
| PVMYKNTGKSEQCIIVNNEDLFFIANKDSFSKDLAKAETIAYNTQNNTIENNFSI | |
| DQLILDNDLSSGIDLPNENTEPFTNFDDIDIPVYIKQSALKKIFVDGDSLFEYLHA | |
| QTFPSNIENLQLTNSLNDALRNNNKVYTFFSTNLVEKANTVVGASLFVNWVKG | |
| VIDDFTSESTQKSTIDKVSDVSIIIPYIGPALNVGNETAKENFKNAFEIGGAAILME | |
| FIPELIVPIVGFFTLESYVGNKGHIIMTISNALKKRDQKWTDMYGLIVSQWLSTV | |
| NTQFYTIKERMYNALNNQSQAIEKIIEDQYNRYSEEDKMNINIDFNDIDFKLNQS | |
| INLAINNIDDFINQCSISYLMNRMIPLAVKKLKDFDDNLKRDLLEYIDTNELYLL | |
| DEVNILKSKVNRHLKDSIPFDLSLYTKDTILIQVFNNYISNISSNAILSLSYRGGRL | |
| IDSSGYGATMNVGSDVIFNDIGNGQFKLNNSENSNITAHQSKFVVYDSMFDNFS | |
| INFWVRTPKYNNNDIQTYLQNEYTIISCIKNDSGWKVSIKGNRIIWTLIDVNAKS | |
| KSIFFEYSIKDNISDYINKWFSITITNDRLGNANIYINGSLKKSEKILNLDRINSSND | |
| IDFKLINCTDTTKFVWIKDFNIFGRELNATEVSSLYWIQSSTNTLKDFWGNPLRY | |
| DTQYYLFNQGMQNIYIKYFSKASMGETAPRTNFNNAAINYQNLYLGLRFIIKKA | |
| SNSRNINNDNIVREGDYIYLNIDNISDESYRVYVLVNSKEIQTQLFLAPINDDPTF | |
| YDVLQIKKYYEKTTYNCQILCEKDTKTFGLFGIGKFVKDYGYVWDTYDNYFCI | |
| SQWYLRRISENINKLRLGCNWQFIPVDEGWTE. | |
| TeNT, accession number P04958, amino acid sequence. | |
| SEQ ID NO: 8 | |
| MPITINNFRYSDPVNNDTIIMMEPPYCKGLDIYYKAFKITDRIWIVPERYEFGTKP | |
| EDFNPPSSLIEGASEYYDPNYLRTDSDKDRFLQTMVKLFNRIKNNVAGEALLDK | |
| IINAIPYLGNSYSLLDKFDTNSNSVSFNLLEQDPSGATTKSAMLTNLIIFGPGPVL | |
| NKNEVRGIVLRVDNKNYFPCRDGFGSIMQMAFCPEYVPTFDNVIENITSLTIGKS | |
| KYFQDPALLLMHELIHVLHGLYGMQVSSHEIIPSKQEIYMQHTYPISAEELFTFG | |
| GQDANLISIDIKNDLYEKTLNDYKAIANKLSQVTSCNDPNIDIDSYKQIYQQKYQ | |
| FDKDSNGQYIVNEDKFQILYNSIMYGFTEIELGKKFNIKTRLSYFSMNHDPVKIP | |
| NLLDDTIYNDTEGFNIESKDLKSEYKGQNMRVNTNAFRNVDGSGLVSKLIGLC | |
| KKIIPPTNIRENLYNRTASLTDLGGELCIKIKNEDLTFIAEKNSFSEEPFQDEIVSY | |
| NTKNKPLNFNYSLDKIIVDYNLQSKITLPNDRTTPVTKGIPYAPEYKSNAASTIEI | |
| HNIDDNTIYQYLYAQKSPTTLQRITMTNSVDDALINSTKIYSYFPSVISKVNQGA | |
| QGILFLQWVRDIIDDFTNESSQKTTIDKISDVSTIVPYIGPALNIVKQGYEGNFIGA | |
| LETTGVVLLLEYIPEITLPVIAALSIAESSTQKEKIIKTIDNFLEKRYEKWIEVYKL | |
| VKAKWLGTVNTQFQKRSYQMYRSLEYQVDAIKKIIDYEYKIYSGPDKEQIADEI | |
| NNLKNKLEEKANKAMININIFMRESSRSFLVNQMINEAKKQLLEFDTQSKNILM | |
| QYIKANSKFIGITELKKLESKINKVFSTPIPFSYSKNLDCWVDNEEDIDVILKKSTI | |
| LNLDINNDIISDISGFNSSVITYPDAQLVPGINGKAIHLVNNESSEVIVHKAMDIE | |
| YNDMFNNFTVSFWLRVPKVSASHLEQYGTNEYSIISSMKKHSLSIGSGWSVSLK | |
| GNNLIWTLKDSAGEVRQITFRDLPDKFNAYLANKWVFITITNDRLSSANLYING | |
| VLMGSAEITGLGAIREDNNITLKLDRCNNNNQYVSIDKFRIFCKALNPKEIEKLY | |
| TSYLSITFLRDFWGNPLRYDTEYYLIPVASSSKDVQLKNITDYMYLTNAPSYTN | |
| GKLNIYYRRLYNGLKFIIKRYTPNNEIDSFVKSGDFIKLYVSYNNNEHIVGYPKD | |
| GNAFNNLDRILRVGYNAPGIPLYKKMEAVKLRDLKTYSVQLKLYDDKNASLG | |
| LVGTHNGQIGNDPNRDILIASNWYFNHLKDKILGCDWYFVPTDEGWTND. | |
| BoNT/A1, DNA. | |
| SEQ ID NO: 9 | |
| ATGCCATTTG TTAATAAACA ATTTAATTAT AAAGATCCTG TAAATGGTGT TGATATTGCT | 60 |
| TATATAAAAA TTCCAAATGC AGGACAAATG CAACCAGTAA AAGCTTTTAA AATTCATAAT | 120 |
| AAAATATGGG TTATTCCAGA AAGAGATACA TTTACAAATC CTGAAGAAGG AGATTTAAAT | 180 |
| CCACCACCAG AAGCAAAACA AGTTCCAGTT TCATATTATG ATTCAACATA TTTAAGTACA | 240 |
| GATAATGAAA AAGATAATTA TTTAAAGGGA GTTACAAAAT TATTTGAGAG AATTTATTCA | 300 |
| ACTGATCTTG GAAGAATGTT GTTAACATCA ATAGTAAGGG GAATACCATT TTGGGGTGGA | 360 |
| AGTACAATAG ATACAGAATT AAAAGTTATT GATACTAATT GTATTAATGT GATACAACCA | 420 |
| GATGGTAGTT ATAGATCAGA AGAACTTAAT CTAGTAATAA TAGGACCCTC AGCTGATATT | 480 |
| ATACAGTTTG AATGTAAAAG CTTTGGACAT GAAGTTTTGA ATCTTACGCG AAATGGTTAT | 540 |
| GGCTCTACTC AATACATTAG ATTTAGCCCA GATTTTACAT TTGGTTTTGA GGAGTCACTT | 600 |
| GAAGTTGATA CAAATCCTCT TTTAGGTGCA GGCAAATTTG CTACAGATCC AGCAGTAACA | 660 |
| TTAGCACATG AACTTATACA TGCTGGACAT AGATTATATG GAATAGCAAT TAATCCAAAT | 720 |
| AGGGTTTTTA AAGTAAATAC TAATGCCTAT TATGAAATGA GTGGGTTAGA AGTAAGCTTT | 780 |
| GAGGAACTTA GAACATTTGG GGGACATGAT GCAAAGTTTA TAGATAGTTT ACAGGAAAAC | 840 |
| GAATTTCGTC TATATTATTA TAATAAGTTT AAAGATATAG CAAGTACACT TAATAAAGCT | 900 |
| AAATCAATAG TAGGTACTAC TGCTTCATTA CAGTATATGA AAAATGTTTT TAAAGAGAAA | 960 |
| TATCTCCTAT CTGAAGATAC ATCTGGAAAA TTTTCGGTAG ATAAATTAAA ATTTGATAAG | 1020 |
| TTATACAAAA TGTTAACAGA GATTTACACA GAGGATAATT TTGTTAAGTT TTTTAAAGTA | 1080 |
| CTTAACAGAA AAACATATTT GAATTTTGAT AAAGCCGTAT TTAAGATAAA TATAGTACCT | 1140 |
| AAGGTAAATT ACACAATATA TGATGGATTT AATTTAAGAA ATACAAATTT AGCAGCAAAC | 1200 |
| TTTAATGGTC AAAATACAGA AATTAATAAT ATGAATTTTA CTAAACTAAA AAATTTTACT | 1260 |
| GGATTGTTTG AATTTTATAA GTTGCTATGT GTAAGAGGGA TAATAACTTC TAAAACTAAA | 1320 |
| TCATTAGATA AAGGATACAA TAAGGCATTA AATGATTTAT GTATCAAAGT TAATAATTGG | 1380 |
| GACTTGTTTT TTAGTCCTTC AGAAGATAAT TTTACTAATG ATCTAAATAA AGGAGAAGAA | 1440 |
| ATTACATCTG ATACTAATAT AGAAGCAGCA GAAGAAAATA TTAGTTTAGA TTTAATACAA | 1500 |
| CAATATTATT TAACCTTTAA TTTTGATAAT GAACCTGAAA ATATTTCAAT AGAAAATCTT | 1560 |
| TCAAGTGACA TTATAGGCCA ATTAGAACTT ATGCCTAATA TAGAAAGATT TCCTAATGGA | 1620 |
| AAAAAGTATG AGTTAGATAA ATATACTATG TTCCATTATC TTCGTGCTCA AGAATTTGAA | 1680 |
| CATGGTAAAT CTAGGATTGC TTTAACAAAT TCTGTTAACG AAGCATTATT AAATCCTAGT | 1740 |
| CGTGTTTATA CATTTTTTTC TTCAGACTAT GTAAAGAAAG TTAATAAAGC TACGGAGGCA | 1800 |
| GCTATGTTTT TAGGCTGGGT AGAACAATTA GTATATGATT TTACCGATGA AACTAGCGAA | 1860 |
| GTAAGTACTA CGGATAAAAT TGCGGATATA ACTATAATTA TTCCATATAT AGGACCTGCT | 1920 |
| TTAAATATAG GTAATATGTT ATATAAAGAT GATTTTGTAG GTGCTTTAAT ATTTTCAGGA | 1980 |
| GCTGTTATTC TGTTAGAATT TATACCAGAG ATTGCAATAC CTGTATTAGG TACTTTTGCA | 2040 |
| CTTGTATCAT ATATTGCGAA TAAGGTTCTA ACCGTTCAAA CAATAGATAA TGCTTTAAGT | 2100 |
| AAAAGAAATG AAAAATGGGA TGAGGTCTAT AAATATATAG TAACAAATTG GTTAGCAAAG | 2160 |
| GTTAATACAC AGATTGATCT AATAAGAAAA AAAATGAAAG AAGCTTTAGA AAATCAAGCA | 2220 |
| GAAGCAACAA AGGCTATAAT AAACTATCAG TATAATCAAT ATACTGAGGA AGAGAAAAAT | 2280 |
| AATATTAATT TTAATATTGA TGATTTAAGT TCGAAACTTA ATGAGTCTAT AAATAAAGCT | 2340 |
| ATGATTAATA TAAATAAATT TTTGAATCAA TGCTCTGTTT CATATTTAAT GAATTCTATG | 2400 |
| ATCCCTTATG GTGTTAAACG GTTAGAAGAT TTTGATGCTA GTCTTAAAGA TGCATTATTA | 2460 |
| AAGTATATAT ATGATAATAG AGGAACTTTA ATTGGTCAAG TAGATAGATT AAAAGATAAA | 2520 |
| GTTAATAATA CACTTAGTAC AGATATACCT TTTCAGCTTT CCAAATACGT AGATAATCAA | 2580 |
| AGATTATTAT CTACATTTAC TGAATATATT AAGAATATTA TTAATACTTC TATATTGAAT | 2640 |
| TTAAGATATG AAAGTAATCA TTTAATAGAC TTATCTAGGT ATGCATCAAA AATAAATATT | 2700 |
| GGTAGTAAAG TAAATTTTGA TCCAATAGAT AAAAATCAAA TTCAATTATT TAATTTAGAA | 2760 |
| AGTAGTAAAA TTGAGGTAAT TTTAAAAAAT GCTATTGTAT ATAATAGTAT GTATGAAAAT | 2820 |
| TTTAGTACTA GCTTTTGGAT AAGAATTCCT AAGTATTTTA ACAGTATAAG TCTAAATAAT | 2880 |
| GAATATACAA TAATAAATTG TATGGAAAAT AATTCAGGAT GGAAAGTATC ACTTAATTAT | 2940 |
| GGTGAAATAA TCTGGACTTT ACAGGATACT CAGGAAATAA AACAAAGAGT AGTTTTTAAA | 3000 |
| TACAGTCAAA TGATTAATAT ATCAGATTAT ATAAACAGAT GGATTTTTGT AACTATCACT | 3060 |
| AATAATAGAT TAAATAACTC TAAAATTTAT ATAAATGGAA GATTAATAGA TCAAAAACCA | 3120 |
| ATTTCAAATT TAGGTAATAT TCATGCTAGT AATAATATAA TGTTTAAATT AGATGGTTGT | 3180 |
| AGAGATACAC ATAGATATAT TTGGATAAAA TATTTTAATC TTTTTGATAA GGAATTAAAT | 3240 |
| GAAAAAGAAA TCAAAGATTT ATATGATAAT CAATCAAATT CAGGTATTTT AAAAGACTTT | 3300 |
| TGGGGTGATT ATTTACAATA TGATAAACCA TACTATATGT TAAATTTATA TGATCCAAAT | 3360 |
| AAATATGTCG ATGTAAATAA TGTAGGTATT AGAGGTTATA TGTATCTTAA AGGGCCTAGA | 3420 |
| GGTAGCGTAA TGACTACAAA CATTTATTTA AATTCAAGTT TGTATAGGGG GACAAAATTT | 3480 |
| ATTATAAAAA AATATGCTTC TGGAAATAAA GATAATATTG TTAGAAATAA TGATCGTGTA | 3540 |
| TATATTAATG TAGTAGTTAA AAATAAAGAA TATAGGTTAG CTACTAATGC ATCACAGGCA | 3600 |
| GGCGTAGAAA AAATACTAAG TGCATTAGAA ATACCTGATG TAGGAAATCT AAGTCAAGTA | 3660 |
| GTAGTAATGA AGTCAAAAAA TGATCAAGGA ATAACAAATA AATGCAAAAT GAATTTACAA | 3720 |
| GATAATAATG GGAATGATAT AGGCTTTATA GGATTTCATC AGTTTAATAA TATAGCTAAA | 3780 |
| CTAGTAGCAA GTAATTGGTA TAATAGACAA ATAGAAAGAT CTAGTAGGAC TTTGGGTTGC | 3840 |
| TCATGGGAAT TTATTCCTGT AGATGATGGA TGGGGAGAAA GGCCACTGTA A. | 3891 |
| BoNVB1, DNA. | |
| SEQ ID NO: 10 | |
| ATGCCAGTTA CAATAAATAA TTTTAATTAT AATGATCCTA TTGATAATAA TAATATTATT | 60 |
| ATGATGGAGC CTCCATTTGC GAGAGGTACG GGGAGATATT ATAAAGCTTT TAAAATCACA | 120 |
| GATCGTATTT GGATAATACC GGAAAGATAT ACTTTTGGAT ATAAACCTGA GGATTTTAAT | 180 |
| AAAAGTTCCG GTATTTTTAA TAGAGATGTT TGTGAATATT ATGATCCAGA TTACTTAAAT | 240 |
| ACTAATGATA AAAAGAATAT ATTTTTACAA ACAATGATCA AGTTATTTAA TAGAATCAAA | 300 |
| TCAAAACCAT TGGGTGAAAA GTTATTAGAG ATGATTATAA ATGGTATACC TTATCTTGGA | 360 |
| GATAGACGTG TTCCACTCGA AGAGTTTAAC ACAAACATTG CTAGTGTAAC TGTTAATAAA | 420 |
| TTAATCAGTA ATCCAGGAGA AGTGGAGCGA AAAAAAGGTA TTTTCGCAAA TTTAATAATA | 480 |
| TTTGGACCTG GGCCAGTTTT AAATGAAAAT GAGACTATAG ATATAGGTAT ACAAAATCAT | 540 |
| TTTGCATCAA GGGAAGGCTT CGGGGGTATA ATGCAAATGA AGTTTTGCCC AGAATATGTA | 600 |
| AGCGTATTTA ATAATGTTCA AGAAAACAAA GGCGCAAGTA TATTTAATAG ACGTGGATAT | 660 |
| TTTTCAGATC CAGCCTTGAT ATTAATGCAT GAACTTATAC ATGTTTTACA TGGATTATAT | 720 |
| GGCATTAAAG TAGATGATTT ACCAATTGTA CCAAATGAAA AAAAATTTTT TATGCAATCT | 780 |
| ACAGATGCTA TACAGGCAGA AGAACTATAT ACATTTGGAG GACAAGATCC CAGCATCATA | 840 |
| ACTCCTTCTA CGGATAAAAG TATCTATGAT AAAGTTTTGC AAAATTTTAG AGGGATAGTT | 900 |
| GATAGACTTA ACAAGGTTTT AGTTTGCATA TCAGATCCTA ACATTAATAT TAATATATAT | 960 |
| AAAAATAAAT TTAAAGATAA ATATAAATTC GTTGAAGATT CTGAGGGAAA ATATAGTATA | 1020 |
| GATGTAGAAA GTTTTGATAA ATTATATAAA AGCTTAATGT TTGGTTTTAC AGAAACTAAT | 1080 |
| ATAGCAGAAA ATTATAAAAT AAAAACTAGA GCTTCTTATT TTAGTGATTC CTTACCACCA | 1140 |
| GTAAAAATAA AAAATTTATT AGATAATGAA ATCTATACTA TAGAGGAAGG GTTTAATATA | 1200 |
| TCTGATAAAG ATATGGAAAA AGAATATAGA GGTCAGAATA AAGCTATAAA TAAACAAGCT | 1260 |
| TATGAAGAAA TTAGCAAGGA GCATTTGGCT GTATATAAGA TACAAATGTG TAAAAGTGTT | 1320 |
| AAAGCTCCAG GAATATGTAT TGATGTTGAT AATGAAGATT TGTTCTTTAT AGCTGATAAA | 1380 |
| AATAGTTTTT CAGATGATTT ATCTAAAAAC GAAAGAATAG AATATAATAC ACAGAGTAAT | 1440 |
| TATATAGAAA ATGACTTCCC TATAAATGAA TTAATTTTAG ATACTGATTT AATAAGTAAA | 1500 |
| ATAGAATTAC CAAGTGAAAA TACAGAATCA CTTACTGATT TTAATGTAGA TGTTCCAGTA | 1560 |
| TATGAAAAAC AACCCGCTAT AAAAAAAATT TTTACAGATG AAAATACCAT CTTTCAATAT | 1620 |
| TTATACTCTC AGACATTTCC TCTAGATATA AGAGATATAA GTTTAACATC TTCATTTGAT | 1680 |
| GATGCATTAT TATTTTCTAA CAAAGTTTAT TCATTTTTTT CTATGGATTA TATTAAAACT | 1740 |
| GCTAATAAAG TGGTAGAAGC AGGATTATTT GCAGGTTGGG TGAAACAGAT AGTAAATGAT | 1800 |
| TTTGTAATCG AAGCTAATAA AAGCAATACT ATGGATAAAA TTGCAGATAT ATCTCTAATT | 1860 |
| GTTCCTTATA TAGGATTAGC TTTAAATGTA GGAAATGAAA CAGCTAAAGG AAATTTTGAA | 1920 |
| AATGCTTTTG AGATTGCAGG AGCCAGTATT CTACTAGAAT TTATACCAGA ACTTTTAATA | 1980 |
| CCTGTAGTTG GAGCCTTTTT ATTAGAATCA TATATTGACA ATAAAAATAA AATTATTAAA | 2040 |
| ACAATAGATA ATGCTTTAAC TAAAAGAAAT GAAAAATGGA GTGATATGTA CGGATTAATA | 2100 |
| GTAGCGCAAT GGCTCTCAAC AGTTAATACT CAATTTTATA CAATAAAAGA GGGAATGTAT | 2160 |
| AAGGCTTTAA ATTATCAAGC ACAAGCATTG GAAGAAATAA TAAAATACAG ATATAATATA | 2220 |
| TATTCTGAAA AAGAAAAGTC AAATATTAAC ATCGATTTTA ATGATATAAA TTCTAAACTT | 2280 |
| AATGAGGGTA TTAACCAAGC TATAGATAAT ATAAATAATT TTATAAATGG ATGTTCTGTA | 2340 |
| TCATATTTAA TGAAAAAAAT GATTCCATTA GCTGTAGAAA AATTACTAGA CTTTGATAAT | 2400 |
| ACTCTCAAAA AAAATTTGTT AAATTATATA GATGAAAATA AATTATATTT GATTGGAAGT | 2460 |
| GCAGAATATG AAAAATCAAA AGTAAATAAA TACTTGAAAA CCATTATGCC GTTTGATCTT | 2520 |
| TCAATATATA CCAATGATAC AATACTAATA GAAATGTTTA ATAAATATAA TAGCGAAATT | 2580 |
| TTAAATAATA TTATCTTAAA TTTAAGATAT AAGGATAATA ATTTAATAGA TTTATCAGGA | 2640 |
| TATGGGGCAA AGGTAGAGGT ATATGATGGA GTCGAGCTTA ATGATAAAAA TCAATTTAAA | 2700 |
| TTAACTAGTT CAGCAAATAG TAAGATTAGA GTGACTCAAA ATCAGAATAT CATATTTAAT | 2760 |
| AGTGTGTTCC TTGATTTTAG CGTTAGCTTT TGGATAAGAA TACCTAAATA TAAGAATGAT | 2820 |
| GGTATACAAA ATTATATTCA TAATGAATAT ACAATAATTA ATTGTATGAA AAATAATTCG | 2880 |
| GGCTGGAAAA TATCTATTAG GGGTAATAGG ATAATATGGA CTTTAATTGA TATAAATGGA | 2940 |
| AAAACCAAAT CGGTATTTTT TGAATATAAC ATAAGAGAAG ATATATCAGA GTATATAAAT | 3000 |
| AGATGGTTTT TTGTAACTAT TACTAATAAT TTGAATAACG CTAAAATTTA TATTAATGGT | 3060 |
| AAGCTAGAAT CAAATACAGA TATTAAAGAT ATAAGAGAAG TTATTGCTAA TGGTGAAATA | 3120 |
| ATATTTAAAT TAGATGGTGA TATAGATAGA ACACAATTTA TTTGGATGAA ATATTTCAGT | 3180 |
| ATTTTTAATA CGGAATTAAG TCAATCAAAT ATTGAAGAAA GATATAAAAT TCAATCATAT | 3240 |
| AGCGAATATT TAAAAGATTT TTGGGGAAAT CCTTTAATGT ACAATAAAGA ATATTATATG | 3300 |
| TTTAATGCGG GGAATAAAAA TTCATATATT AAACTAAAGA AAGATTCACC TGTAGGTGAA | 3360 |
| ATTTTAACAC GTAGCAAATA TAATCAAAAT TCTAAATATA TAAATTATAG AGATTTATAT | 3420 |
| ATTGGAGAAA AATTTATTAT AAGAAGAAAG TCAAATTCTC AATCTATAAA TGATGATATA | 3480 |
| GTTAGAAAAG AAGATTATAT ATATCTAGAT TTTTTTAATT TAAATCAAGA GTGGAGAGTA | 3540 |
| TATACCTATA AATATTTTAA GAAAGAGGAA GAAAAATTGT TTTTAGCTCC TATAAGTGAT | 3600 |
| TCTGATGAGT TTTACAATAC TATACAAATA AAAGAATATG ATGAACAGCC AACATATAGT | 3660 |
| TGTCAGTTGC TTTTTAAAAA AGATGAAGAA AGTACTGATG AGATAGGATT GATTGGTATT | 3720 |
| CATCGTTTCT ACGAATCTGG AATTGTATTT GAAGAGTATA AAGATTATTT TTGTATAAGT | 3780 |
| AAATGGTACT TAAAAGAGGT AAAAAGGAAA CCATATAATT TAAAATTGGG ATGTAATTGG | 3840 |
| CAGTTTATTC CTAAAGATGA AGGGTGGACT GAATAA. | 3876 |
| BoNI7C1, DNA. | |
| SEQ ID NO: 11 | |
| ATGCCAATAA CAATTAACAA CTTTAATTAT TCAGATCCTG TTGATAATAA AAATATTTTA | 60 |
| TATTTAGATA CTCATTTAAA TACACTAGCT AATGAGCCTG AAAAAGCCTT TCGCATTACA | 120 |
| GGAAATATAT GGGTAATACC TGATAGATTT TCAAGAAATT CTAATCCAAA TTTAAATAAA | 180 |
| CCTCCTCGAG TTACAAGCCC TAAAAGTGGT TATTATGATC CTAATTATTT GAGTACTGAT | 240 |
| TCTGACAAAG ATACATTTTT AAAAGAAATT ATAAAGTTAT TTAAAAGAAT TAATTCTAGA | 300 |
| GAAATAGGAG AAGAATTAAT ATATAGACTT TCGACAGATA TACCCTTTCC TGGGAATAAC | 360 |
| AATACTCCAA TTAATACTTT TGATTTTGAT GTAGATTTTA ACAGTGTTGA TGTTAAAACT | 420 |
| AGACAAGGTA ACAACTGGGT TAAAACTGGT AGCATAAATC CTAGTGTTAT AATAACTGGA | 480 |
| CCTAGAGAAA ACATTATAGA TCCAGAAACT TCTACGTTTA AATTAACTAA CAATACTTTT | 540 |
| GCGGCACAAG AAGGATTTGG TGCTTTATCA ATAATTTCAA TATCACCTAG ATTTATGCTA | 600 |
| ACATATAGTA ATGCAACTAA TGATGTAGGA GAGGGTAGAT TTTCTAAGTC TGAATTTTGC | 660 |
| ATGGATCCAA TACTAATTTT AATGCATGAA CTTAATCATG CAATGCATAA TTTATATGGA | 720 |
| ATAGCTATAC CAAATGATCA AACAATTTCA TCTGTAACTA GTAATATTTT TTATTCTCAA | 780 |
| TATAATGTGA AATTAGAGTA TGCAGAAATA TATGCATTTG GAGGTCCAAC TATAGACCTT | 840 |
| ATTCCTAAAA GTGCAAGGAA ATATTTTGAG GAAAAGGCAT TGGATTATTA TAGATCTATA | 900 |
| GCTAAAAGAC TTAATAGTAT AACTACTGCA AATCCTTCAA GCTTTAATAA ATATATAGGG | 960 |
| GAATATAAAC AGAAACTTAT TAGAAAGTAT AGATTCGTAG TAGAATCTTC AGGTGAAGTT | 1020 |
| ACAGTAAATC GTAATAAGTT TGTTGAGTTA TATAATGAAC TTACACAAAT ATTTACAGAA | 1080 |
| TTTAACTACG CTAAAATATA TAATGTACAA AATAGGAAAA TATATCTTTC AAATGTATAT | 1140 |
| ACTCCGGTTA CGGCGAATAT ATTAGACGAT AATGTTTATG ATATACAAAA TGGATTTAAT | 1200 |
| ATACCTAAAA GTAATTTAAA TGTACTATTT ATGGGTCAAA ATTTATCTCG AAATCCAGCA | 1260 |
| TTAAGAAAAG TCAATCCTGA AAATATGCTT TATTTATTTA CAAAATTTTG TCATAAAGCA | 1320 |
| ATAGATGGTA GATCATTATA TAATAAAACA TTAGATTGTA GAGAGCTTTT AGTTAAAAAT | 1380 |
| ACTGACTTAC CCTTTATAGG TGATATTAGT GATGTTAAAA CTGATATATT TTTAAGAAAA | 1440 |
| GATATTAATG AAGAAACTGA AGTTATATAC TATCCGGACA ATGTTTCAGT AGATCAAGTT | 1500 |
| ATTCTCAGTA AGAATACCTC AGAACATGGA CAACTAGATT TATTATACCC TAGTATTGAC | 1560 |
| AGTGAGAGTG AAATATTACC AGGGGAGAAT CAAGTCTTTT ATGATAATAG AACTCAAAAT | 1620 |
| GTTGATTATT TGAATTCTTA TTATTACCTA GAATCTCAAA AACTAAGTGA TAATGTTGAA | 1680 |
| GATTTTACTT TTACGAGATC AATTGAGGAG GCTTTGGATA ATAGTGCAAA AGTATATACT | 1740 |
| TACTTTCCTA CACTAGCTAA TAAAGTAAAT GCGGGTGTTC AAGGTGGTTT ATTTTTAATG | 1800 |
| TGGGCAAATG ATGTAGTTGA AGATTTTACT ACAAATATTC TAAGAAAAGA TACATTAGAT | 1860 |
| AAAATATCAG ATGTATCAGC TATTATTCCC TATATAGGAC CCGCATTAAA TATAAGTAAT | 1920 |
| TCTGTAAGAA GAGGAAATTT TACTGAAGCA TTTGCAGTTA CTGGTGTAAC TATTTTATTA | 1980 |
| GAAGCATTTC CTGAATTTAC AATACCTGCA CTTGGTGCAT TTGTGATTTA TAGTAAGGTT | 2040 |
| CAAGAAAGAA ACGAGATTAT TAAAACTATA GATAATTGTT TAGAACAAAG GATTAAGAGA | 2100 |
| TGGAAAGATT CATATGAATG GATGATGGGA ACGTGGTTAT CCAGGATTAT TACTCAATTT | 2160 |
| AATAATATAA GTTATCAAAT GTATGATTCT TTAAATTATC AGGCAGGTGC AATCAAAGCT | 2220 |
| AAAATAGATT TAGAATATAA AAAATATTCA GGAAGTGATA AAGAAAATAT AAAAAGTCAA | 2280 |
| GTTGAAAATT TAAAAAATAG TTTAGATGTA AAAATTTCGG AAGCAATGAA TAATATAAAT | 2340 |
| AAATTTATAC GAGAATGTTC CGTAACATAT TTATTTAAAA ATATGTTACC TAAAGTAATT | 2400 |
| GATGAATTAA ATGAGTTTGA TCGAAATACT AAAGCAAAAT TAATTAATCT TATAGATAGT | 2460 |
| CATAATATTA TTCTAGTTGG TGAAGTAGAT AAATTAAAAG CAAAAGTAAA TAATAGCTTT | 2520 |
| CAAAATACAA TACCCTTTAA TATTTTTTCA TATACTAATA ATTCTTTATT AAAAGATATA | 2580 |
| ATTAATGAAT ATTTCAATAA TATTAATGAT TCAAAAATTT TGAGCCTACA AAACAGAAAA | 2640 |
| AATACTTTAG TGGATACATC AGGATATAAT GCAGAAGTGA GTGAAGAAGG CGATGTTCAG | 2700 |
| CTTAATCCAA TATTTCCATT TGACTTTAAA TTAGGTAGTT CAGGGGAGGA TAGAGGTAAA | 2760 |
| GTTATAGTAA CCCAGAATGA AAATATTGTA TATAATTCTA TGTATGAAAG TTTTAGCATT | 2820 |
| AGTTTTTGGA TTAGAATAAA TAAATGGGTA AGTAATTTAC CTGGATATAC TATAATTGAT | 2880 |
| AGTGTTAAAA ATAACTCAGG TTGGAGTATA GGTATTATTA GTAATTTTTT AGTATTTACT | 2940 |
| TTAAAACAAA ATGAAGATAG TGAACAAAGT ATAAATTTTA GTTATGATAT ATCAAATAAT | 3000 |
| GCTCCTGGAT ACAATAAATG GTTTTTTGTA ACTGTTACTA ACAATATGAT GGGAAATATG | 3060 |
| AAGATTTATA TAAATGGAAA ATTAATAGAT ACTATAAAAG TTAAAGAACT AACTGGAATT | 3120 |
| AATTTTAGCA AAACTATAAC ATTTGAAATA AATAAAATTC CAGATACCGG TTTGATTACT | 3180 |
| TCAGATTCTG ATAACATCAA TATGTGGATA AGAGATTTTT ATATATTTGC TAAAGAATTA | 3240 |
| GATGGTAAAG ATATTAATAT ATTATTTAAT AGCTTGCAAT ATACTAATGT TGTAAAAGAT | 3300 |
| TATTGGGGAA ATGATTTAAG ATATAATAAA GAATATTATA TGGTTAATAT AGATTATTTA | 3360 |
| AATAGATATA TGTATGCGAA CTCACGACAA ATTGTTTTTA ATACACGTAG AAATAATAAT | 3420 |
| GACTTCAATG AAGGATATAA AATTATAATA AAAAGAATCA GAGGAAATAC AAATGATACT | 3480 |
| AGAGTACGAG GAGGAGATAT TTTATATTTT GATATGACAA TTAATAACAA AGCATATAAT | 3540 |
| TTGTTTATGA AGAATGAAAC TATGTATGCA GATAATCATA GTACTGAAGA TATATATGCT | 3600 |
| ATAGGTTTAA GAGAACAAAC AAAGGATATA AATGATAATA TTATATTTCA AATACAACCA | 3660 |
| ATGAATAATA CTTATTATTA CGCATCTCAA ATATTTAAAT CAAATTTTAA TGGAGAAAAT | 3720 |
| ATTTCTGGAA TATGTTCAAT AGGTACTTAT CGTTTTAGAC TTGGAGGTGA TTGGTATAGA | 3780 |
| CACAATTATT TGGTGCCTAC TGTGAAGCAA GGAAATTATG CTTCATTATT AGAATCAACA | 3840 |
| TCAACTCATT GGGGTTTTGT ACCTGTAAGT GAATAA. | 3876 |
| BoNT/D, DNA. | |
| SEQ ID NO: 12 | |
| ATGACATGGC CAGTAAAAGA TTTTAATTAT AGTGATCCTG TTAATGACAA TGATATATTA | 60 |
| TATTTAAGAA TACCACAAAA TAAGTTAATT ACTACACCTG TAAAAGCTTT TATGATTACT | 120 |
| CAAAATATTT GGGTAATACC AGAAAGATTT TCATCAGATA CTAATCCAAG TTTAAGTAAA | 180 |
| CCGCCCAGAC CTACTTCAAA GTATCAAAGT TATTATGATC CTAGTTATTT ATCTACTGAT | 240 |
| GAACAAAAAG ATACATTTTT AAAAGGGATT ATAAAATTAT TTAAAAGAAT TAATGAAAGA | 300 |
| GATATAGGAA AAAAATTAAT AAATTATTTA GTAGTTGGTT CACCTTTTAT GGGAGATTCA | 360 |
| AGTACGCCTG AAGATACATT TGATTTTACA CGTCATACTA CTAATATTGC AGTTGAAAAG | 420 |
| TTTGAAAATG GTAGTTGGAA AGTAACAAAT ATTATAACAC CAAGTGTATT GATATTTGGA | 480 |
| CCACTTCCTA ATATATTAGA CTATACAGCA TCCCTTACAT TGCAAGGACA ACAATCAAAT | 540 |
| CCATCATTTG AAGGGTTTGG AACATTATCT ATACTAAAAG TAGCACCTGA ATTTTTGTTA | 600 |
| ACATTTAGTG ATGTAACATC TAATCAAAGT TCAGCTGTAT TAGGCAAATC TATATTTTGT | 660 |
| ATGGATCCAG TAATAGCTTT AATGCATGAG TTAACACATT CTTTGCATCA ATTATATGGA | 720 |
| ATAAATATAC CATCTGATAA AAGGATTCGT CCACAAGTTA GCGAGGGATT TTTCTCTCAA | 780 |
| GATGGACCCA ACGTACAATT TGAGGAATTA TATACATTTG GAGGATTAGA TGTTGAAATA | 840 |
| ATACCTCAAA TTGAAAGATC ACAATTAAGA GAAAAAGCAT TAGGTCACTA TAAAGATATA | 900 |
| GCGAAAAGAC TTAATAATAT TAATAAAACT ATTCCTTCTA GTTGGATTAG TAATATAGAT | 960 |
| AAATATAAAA AAATATTTTC TGAAAAGTAT AATTTTGATA AAGATAATAC AGGAAATTTT | 1020 |
| GTTGTAAATA TTGATAAATT CAATAGCTTA TATTCAGACT TGACTAATGT TATGTCAGAA | 1080 |
| GTTGTTTATT CTTCGCAATA TAATGTTAAA AACAGGACTC ATTATTTTTC AAGGCATTAT | 1140 |
| CTACCTGTAT TTGCAAATAT ATTAGATGAT AATATTTATA CTATAAGAGA TGGTTTTAAT | 1200 |
| TTAACAAATA AAGGTTTTAA TATAGAAAAT TCGGGTCAGA ATATAGAAAG GAATCCTGCA | 1260 |
| CTACAAAAGC TTAGTTCAGA AAGTGTAGTA GATTTATTTA CAAAAGTATG TTTAAGATTA | 1320 |
| ACAAAAAATA GTAGAGATGA TTCAACATGT ATTAAAGTTA AAAATAATAG ATTACCTTAT | 1380 |
| GTAGCTGATA AAGATAGCAT TTCACAAGAA ATATTTGAAA ATAAAATTAT TACAGATGAG | 1440 |
| ACTAATGTAC AAAATTATTC AGATAAATTT TCATTAGATG AATCTATTTT AGATGGGCAA | 1500 |
| GTTCCTATTA ATCCTGAAAT AGTAGATCCA CTATTACCCA ATGTTAATAT GGAACCTTTA | 1560 |
| AATCTTCCAG GTGAAGAAAT AGTATTTTAT GATGATATTA CTAAATATGT TGATTATTTA | 1620 |
| AATTCTTATT ATTATTTGGA ATCTCAAAAA TTAAGTAATA ATGTTGAAAA TATTACTCTT | 1680 |
| ACAACTTCAG TTGAAGAAGC ATTAGGTTAT AGCAATAAGA TATACACATT TTTACCTAGC | 1740 |
| TTAGCTGAAA AAGTGAATAA AGGTGTTCAA GCAGGTTTAT TCTTAAATTG GGCGAATGAA | 1800 |
| GTAGTTGAGG ATTTTACTAC AAATATTATG AAGAAAGATA CATTGGATAA AATATCAGAT | 1860 |
| GTATCAGTAA TAATTCCATA TATAGGACCT GCCTTAAATA TAGGAAATTC AGCATTAAGG | 1920 |
| GGAAATTTTA ATCAAGCATT TGCAACAGCT GGTGTAGCTT TTTTATTAGA GGGATTTCCA | 1980 |
| GAGTTTACTA TACCTGCACT CGGTGTATTT ACCTTTTATA GTTCTATTCA AGAAAGAGAG | 2040 |
| AAAATTATTA AAACTATAGA AAATTGTTTG GAACAAAGAG TTAAGAGATG GAAAGATTCA | 2100 |
| TATCAATGGA TGGTATCAAA TTGGTTGTCA AGAATTACTA CTCAATTTAA TCATATAAAT | 2160 |
| TATCAAATGT ATGATTCTTT AAGTTATCAG GCAGATGCAA TCAAAGCTAA AATAGATTTA | 2220 |
| GAATATAAAA AATACTCAGG AAGTGATAAA GAAAATATAA AAAGTCAAGT TGAAAATTTA | 2280 |
| AAAAATAGTT TAGATGTAAA AATTTCGGAA GCAATGAATA ATATAAATAA ATTTATACGA | 2340 |
| GAATGTTCTG TAACATACTT ATTTAAAAAT ATGCTCCCTA AAGTAATTGA CGAATTAAAT | 2400 |
| AAGTTTGATT TAAGAACTAA AACAGAATTA ATTAATCTTA TAGATAGTCA TAATATTATT | 2460 |
| CTAGTTGGTG AAGTAGATAG ATTAAAAGCA AAAGTAAATG AGAGTTTTGA AAATACAATG | 2520 |
| CCTTTTAATA TTTTTTCATA TACTAATAAT TCTTTATTAA AAGATATAAT TAATGAATAT | 2580 |
| TTCAATAGTA TTAATGATTC AAAAATTTTG AGCTTACAAA ACAAAAAAAA TGCTTTAGTG | 2640 |
| GATACATCAG GATATAATGC AGAAGTGAGG GTAGGAGATA ATGTTCAACT TAATACGATA | 2700 |
| TATACAAATG ACTTTAAATT AAGTAGTTCA GGAGATAAAA TTATAGTAAA TTTAAATAAT | 2760 |
| AATATTTTAT ATAGCGCTAT TTATGAGAAC TCTAGTGTTA GTTTTTGGAT TAAGATATCT | 2820 |
| AAAGATTTAA CTAATTCTCA TAATGAATAT ACAATAATTA ACAGTATAGA ACAAAATTCT | 2880 |
| GGGTGGAAAT TATGTATTAG GAATGGCAAT ATAGAATGGA TTTTACAAGA TGTTAATAGA | 2940 |
| AAGTATAAAA GTTTAATTTT TGATTATAGT GAATCATTAA GTCATACAGG ATATACAAAT | 3000 |
| AAATGGTTTT TTGTTACTAT AACTAATAAT ATAATGGGGT ATATGAAACT TTATATAAAT | 3060 |
| GGAGAATTAA AGCAGAGTCA AAAAATTGAA GATTTAGATG AGGTTAAGTT AGATAAAACC | 3120 |
| ATAGTATTTG GAATAGATGA GAATATAGAT GAGAATCAGA TGCTTTGGAT TAGAGATTTT | 3180 |
| AATATTTTTT CTAAAGAATT AAGTAATGAA GATATTAATA TTGTATATGA GGGACAAATA | 3240 |
| TTAAGAAATG TTATTAAAGA TTATTGGGGA AATCCTTTGA AGTTTGATAC AGAATATTAT | 3300 |
| ATTATTAATG ATAATTATAT AGATAGGTAT ATAGCACCTG AAAGTAATGT ACTTGTACTT | 3360 |
| GTTCAGTATC CAGATAGATC TAAATTATAT ACTGGAAATC CTATTACTAT TAAATCAGTA | 3420 |
| TCTGATAAGA ATCCTTATAG TAGAATTTTA AATGGAGATA ATATAATTCT TCATATGTTA | 3480 |
| TATAATAGTA GGAAATATAT GATAATAAGA GATACTGATA CAATATATGC AACACAAGGA | 3540 |
| GGAGAGTGTT CACAAAATTG TGTATATGCA TTAAAATTAC AGAGTAATTT AGGTAATTAT | 3600 |
| GGTATAGGTA TATTTAGTAT AAAAAATATT GTATCTAAAA ATAAATATTG TAGTCAAATT | 3660 |
| TTCTCTAGTT TTAGGGAAAA TACAATGCTT CTAGCAGATA TATATAAACC TTGGAGATTT | 3720 |
| TCTTTTAAAA ATGCATACAC GCCAGTTGCA GTAACTAATT ATGAAACAAA ACTATTATCA | 3780 |
| ACTTCATCTT TTTGGAAATT TATTTCTAGG GATCCAGGAT GGGTAGAGTA A. | 3831 |
| BoNT/E1, DNA. | |
| SEQ ID NO: 13 | |
| ATGCCAAAAA TTAATAGTTT TAATTATAAT GATCCTGTTA ATGATAGAAC AATTTTATAT | 60 |
| ATTAAACCAG GCGGTTGTCA AGAATTTTAT AAATCATTTA ATATTATGAA AAATATTTGG | 120 |
| ATAATTCCAG AGAGAAATGT AATTGGTACA ACCCCCCAAG ATTTTCATCC GCCTACTTCA | 180 |
| TTAAAAAATG GAGATAGTAG TTATTATGAC CCTAATTATT TACAAAGTGA TGAAGAAAAG | 240 |
| GATAGATTTT TAAAAATAGT CACAAAAATA TTTAATAGAA TAAATAATAA TCTTTCAGGA | 300 |
| GGGATTTTAT TAGAAGAACT GTCAAAAGCT AATCCATATT TAGGGAATGA TAATACTCCA | 360 |
| GATAATCAAT TCCATATTGG TGATGCATCA GCAGTTGAGA TTAAATTCTC AAATGGTAGC | 420 |
| CAAGACATAC TATTACCTAA TGTTATTATA ATGGGAGCAG AGCCTGATTT ATTTGAAACT | 480 |
| AACAGTTCCA ATATTTCTCT AAGAAATAAT TATATGCCAA GCAATCACCG TTTTGGATCA | 540 |
| ATAGCTATAG TAACATTCTC ACCTGAATAT TCTTTTAGAT TTAATGATAA TTGTATGAAT | 600 |
| GAATTTATTC AAGATCCTGC TCTTACATTA ATGCATGAAT TAATACATTC ATTACATGGA | 660 |
| CTATATGGGG CTAAAGGGAT TACTACAAAG TATACTATAA CACAAAAACA AAATCCCCTA | 720 |
| ATAACAAATA TAAGAGGTAC AAATATTGAA GAATTCTTAA CTTTTGGAGG TACTGATTTA | 780 |
| AACATTATTA CTAGTGCTCA GTCCAATGAT ATCTATACTA ATCTTCTAGC TGATTATAAA | 840 |
| AAAATAGCGT CTAAACTTAG CAAAGTACAA GTATCTAATC CACTACTTAA TCCTTATAAA | 900 |
| GATGTTTTTG AAGCAAAGTA TGGATTAGAT AAAGATGCTA GCGGAATTTA TTCGGTAAAT | 960 |
| ATAAACAAAT TTAATGATAT TTTTAAAAAA TTATACAGCT TTACGGAATT TGATTTACGA | 1020 |
| ACTAAATTTC AAGTTAAATG TAGGCAAACT TATATTGGAC AGTATAAATA CTTCAAACTT | 1080 |
| TCAAACTTGT TAAATGATTC TATTTATAAT ATATCAGAAG GCTATAATAT AAATAATTTA | 1140 |
| AAGGTAAATT TTAGAGGACA GAATGCAAAT TTAAATCCTA GAATTATTAC ACCAATTACA | 1200 |
| GGTAGAGGAC TAGTAAAAAA AATCATTAGA TTTTGTAAAA ATATTGTTTC TGTAAAAGGC | 1260 |
| ATAAGGAAAT CAATATGTAT CGAAATAAAT AATGGTGAGT TATTTTTTGT GGCTTCCGAG | 1320 |
| AATAGTTATA ATGATGATAA TATAAATACT CCTAAAGAAA TTGACGATAC AGTAACTTCA | 1380 |
| AATAATAATT ATGAAAATGA TTTAGATCAG GTTATTTTAA ATTTTAATAG TGAATCAGCA | 1440 |
| CCTGGACTTT CAGATGAAAA ATTAAATTTA ACTATCCAAA ATGATGCTTA TATACCAAAA | 1500 |
| TATGATTCTA ATGGAACAAG TGATATAGAA CAACATGATG TTAATGAACT TAATGTATTT | 1560 |
| TTCTATTTAG ATGCACAGAA AGTGCCCGAA GGTGAAAATA ATGTCAATCT CACCTCTTCA | 1620 |
| ATTGATACAG CATTATTAGA ACAACCTAAA ATATATACAT TTTTTTCATC AGAATTTATT | 1680 |
| AATAATGTCA ATAAACCTGT GCAAGCAGCA TTATTTGTAA GCTGGATACA ACAAGTGTTA | 1740 |
| GTAGATTTTA CTACTGAAGC TAACCAAAAA AGTACTGTTG ATAAAATTGC AGATATTTCT | 1800 |
| ATAGTTGTTC CATATATAGG TCTTGCTTTA AATATAGGAA ATGAAGCACA AAAAGGAAAT | 1860 |
| TTTAAAGATG CACTTGAATT ATTAGGAGCA GGTATTTTAT TAGAATTTGA ACCCGAGCTT | 1920 |
| TTAATTCCTA CAATTTTAGT ATTCACGATA AAATCTTTTT TAGGTTCATC TGATAATAAA | 1980 |
| AATAAAGTTA TTAAAGCAAT AAATAATGCA TTGAAAGAAA GAGATGAAAA ATGGAAAGAA | 2040 |
| GTATATAGTT TTATAGTATC GAATTGGATG ACTAAAATTA ATACACAATT TAATAAAAGA | 2100 |
| AAAGAACAAA TGTATCAAGC TTTACAAAAT CAAGTAAATG CAATTAAAAC AATAATAGAA | 2160 |
| TCTAAGTATA ATAGTTATAC TTTAGAGGAA AAAAATGAGC TTACAAATAA ATATGATATT | 2220 |
| AAGCAAATAG AAAATGAACT TAATCAAAAG GTTTCTATAG CAATGAATAA TATAGACAGG | 2280 |
| TTCTTAACTG AAAGTTCTAT ATCCTATTTA ATGAAAATAA TAAATGAAGT AAAAATTAAT | 2340 |
| AAATTAAGAG AATATGATGA GAATGTCAAA ACGTATTTAT TGAATTATAT TATACAACAT | 2400 |
| GGATCAATCT TGGGAGAGAG TCAGCAAGAA CTAAATTCTA TGGTAACTGA TACCCTAAAT | 2460 |
| AATAGTATTC CTTTTAAGCT TTCTTCTTAT ACAGATGATA AAATTTTAAT TTCATATTTT | 2520 |
| AATAAATTCT TTAAGAGAAT TAAAAGTAGT TCAGTTTTAA ATATGAGATA TAAAAATGAT | 2580 |
| AAATACGTAG ATACTTCAGG ATATGATTCA AATATAAATA TTAATGGAGA TGTATATAAA | 2640 |
| TATCCAACTA ATAAAAATCA ATTTGGAATA TATAATGATA AACTTAGTGA AGTTAATATA | 2700 |
| TCTCAAAATG ATTACATTAT ATATGATAAT AAATATAAAA ATTTTAGTAT TAGTTTTTGG | 2760 |
| GTAAGAATTC CTAACTATGA TAATAAGATA GTAAATGTTA ATAATGAATA CACTATAATA | 2820 |
| AATTGTATGA GAGATAATAA TTCAGGATGG AAAGTATCTC TTAATCATAA TGAAATAATT | 2880 |
| TGGACATTCG AAGATAATCG AGGAATTAAT CAAAAATTAG CATTTAACTA TGGTAACGCA | 2940 |
| AATGGTATTT CTGATTATAT AAATAAGTGG ATTTTTGTAA CTATAACTAA TGATAGATTA | 3000 |
| GGAGATTCTA AACTTTATAT TAATGGAAAT TTAATAGATC AAAAATCAAT TTTAAATTTA | 3060 |
| GGTAATATTC ATGTTAGTGA CAATATATTA TTTAAAATAG TTAATTGTAG TTATACAAGA | 3120 |
| TATATTGGTA TTAGATATTT TAATATTTTT GATAAAGAAT TAGATGAAAC AGAAATTCAA | 3180 |
| ACTTTATATA GCAATGAACC TAATACAAAT ATTTTGAAGG ATTTTTGGGG AAATTATTTG | 3240 |
| CTTTATGACA AAGAATACTA TTTATTAAAT GTGTTAAAAC CAAATAACTT TATTGATAGG | 3300 |
| AGAAAAGATT CTACTTTAAG CATTAATAAT ATAAGAAGCA CTATTCTTTT AGCTAATAGA | 3360 |
| TTATATAGTG GAATAAAAGT TAAAATACAA AGAGTTAATA ATAGTAGTAC TAACGATAAT | 3420 |
| CTTGTTAGAA AGAATGATCA GGTATATATT AATTTTGTAG CCAGCAAAAC TCACTTATTT | 3480 |
| CCATTATATG CTGATACAGC TACCACAAAT AAAGAGAAAA CAATAAAAAT ATCATCATCT | 3540 |
| GGCAATAGAT TTAATCAAGT AGTAGTTATG AATTCAGTAG GAAATTGTAC AATGAATTTT | 3600 |
| AAAAATAATA ATGGAAATAA TATTGGGTTG TTAGGTTTCA AGGCAGATAC TGTCGTTGCT | 3660 |
| AGTACTTGGT ATTATACACA TATGAGAGAT CATACAAACA GCAATGGATG TTTTTGGAAC | 3720 |
| TTTATTTCTG AAGAACATGG ATGGCAAGAA AAATAA. | 3756 |
| BoNT/F1, DNA. | |
| SEQ ID NO: 14 | |
| ATGCCAGTTG TAATAAATAG TTTTAATTAT AATGACCCTG TTAATGATGA TACAATTTTA | 60 |
| TACATGCAGA TACCATATGA AGAAAAAAGT AAAAAATATT ATAAAGCTTT TGAGATTATG | 120 |
| CGTAATGTTT GGATAATTCC TGAGAGAAAT ACAATAGGAA CGGATCCTAG TGATTTTGAT | 180 |
| CCACCGGCTT CATTAGAGAA CGGAAGCAGT GCTTATTATG ATCCTAATTA TTTAACCACT | 240 |
| GATGCTGAAA AAGATAGATA TTTAAAAACA ACGATAAAAT TATTTAAGAG AATTAATAGT | 300 |
| AATCCTGCAG GGGAAGTTTT GTTACAAGAA ATATCATATG CTAAACCATA TTTAGGAAAT | 360 |
| GAACACACGC CAATTAATGA ATTCCATCCA GTTACTAGAA CTACAAGTGT TAATATAAAA | 420 |
| TCATCAACTA ATGTTAAAAG TTCAATAATA TTGAATCTTC TTGTATTGGG AGCAGGACCT | 480 |
| GATATATTTG AAAATTCTTC TTACCCCGTT AGAAAACTAA TGGATTCAGG TGGAGTTTAT | 540 |
| GACCCAAGTA ATGATGGTTT TGGATCAATT AATATCGTGA CATTTTCACC TGAATATGAA | 600 |
| TATACTTTTA ATGATATTAG TGGAGGGTAT AACAGTAGTA CAGAATCATT TATTGCAGAT | 660 |
| CCTGCAATTT CACTAGCTCA TGAATTGATA CATGCACTGC ATGGATTATA CGGGGCTAGG | 720 |
| GGAGTTACTT ATAAAGAGAC TATAAAAGTA AAGCAAGCAC CTCTTATGAT AGCCGAAAAA | 780 |
| CCCATAAGGC TAGAAGAATT TTTAACCTTT GGAGGTCAGG ATTTAAATAT TATTACTAGT | 840 |
| GCTATGAAGG AAAAAATATA TAACAATCTT TTAGCTAACT ATGAAAAAAT AGCTACTAGA | 900 |
| CTTAGTAGAG TTAATAGTGC TCCTCCTGAA TATGATATTA ATGAATATAA AGATTATTTT | 960 |
| CAATGGAAGT ATGGGCTAGA TAAAAATGCT GATGGAAGTT ATACTGTAAA TGAAAATAAA | 1020 |
| TTTAATGAAA TTTATAAAAA ATTATATAGC TTTACAGAGA TTGACTTAGC AAATAAATTT | 1080 |
| AAAGTAAAAT GTAGAAATAC TTATTTTATT AAATATGGAT TTTTAAAAGT TCCAAATTTG | 1140 |
| TTAGATGATG ATATTTATAC TGTATCAGAG GGGTTTAATA TAGGTAATTT AGCAGTAAAC | 1200 |
| AATCGCGGAC AAAATATAAA GTTAAATCCT AAAATTATTG ATTCCATTCC AGATAAAGGT | 1260 |
| CTAGTGGAAA AGATCGTTAA ATTTTGTAAG AGCGTTATTC CTAGAAAAGG TACAAAGGCG | 1320 |
| CCACCGCGAC TATGCATTAG AGTAAATAAT AGGGAGTTAT TTTTTGTAGC TTCAGAAAGT | 1380 |
| AGCTATAATG AAAATGATAT TAATACACCT AAAGAAATTG ACGATACAAC AAATCTAAAT | 1440 |
| AATAATTATA GAAATAATTT AGATGAAGTT ATTTTAGATT ATAATAGTGA GACAATACCT | 1500 |
| CAAATATCAA ATCAAACATT AAATACACTT GTACAAGACG ATAGTTATGT GCCAAGATAT | 1560 |
| GATTCTAATG GAACAAGTGA AATAGAGGAA CATAATGTTG TTGACCTTAA TGTATTTTTC | 1620 |
| TATTTACATG CACAAAAAGT ACCAGAAGGT GAAACTAATA TAAGTTTAAC TTCTTCAATT | 1680 |
| GATACGGCAT TATCAGAAGA ATCGCAAGTA TATACATTCT TTTCTTCAGA GTTTATTAAT | 1740 |
| ACTATCAATA AACCTGTACA CGCAGCACTA TTTATAAGTT GGATAAATCA AGTAATAAGA | 1800 |
| GATTTTACTA CTGAAGCTAC ACAAAAAAGT ACTTTTGATA AGATTGCAGA CATATCTTTA | 1860 |
| GTTGTACCAT ATGTAGGTCT TGCTTTAAAT ATAGGTAATG AGGTACAAAA AGAAAATTTT | 1920 |
| AAGGAGGCAT TTGAATTATT AGGAGCGGGT ATTTTATTAG AATTTGTGCC AGAGCTTTTA | 1980 |
| ATTCCTACAA TTTTAGTGTT TACAATAAAA TCCTTTATAG GTTCATCTGA GAATAAAAAT | 2040 |
| AAAATCATTA AAGCAATAAA TAATTCATTA ATGGAAAGAG AAACAAAGTG GAAAGAAATA | 2100 |
| TATAGTTGGA TAGTATCAAA TTGGCTTACT AGAATTAATA CACAATTTAA TAAAAGAAAA | 2160 |
| GAACAAATGT ATCAAGCTTT GCAAAATCAA GTAGATGCAA TAAAAACAGT AATAGAATAT | 2220 |
| AAATATAATA ATTATACTTC AGATGAGAGA AATAGACTTG AATCTGAATA TAATATCAAT | 2280 |
| AATATAAGAG AAGAATTGAA CAAAAAAGTT TCTTTAGCAA TGGAAAATAT AGAGAGATTT | 2340 |
| ATAACAGAGA GTTCTATATT TTATTTAATG AAGTTAATAA ATGAAGCCAA AGTTAGTAAA | 2400 |
| TTAAGAGAAT ATGATGAAGG CGTTAAGGAA TATTTGCTAG ACTATATTTC AGAACATAGA | 2460 |
| TCAATTTTAG GAAATAGTGT ACAAGAATTA AATGATTTAG TGACTAGTAC TCTGAATAAT | 2520 |
| AGTATTCCAT TTGAACTTTC TTCATATACT AATGATAAAA TTCTAATTTT ATATTTTAAT | 2580 |
| AAATTATATA AAAAAATTAA AGATAACTCT ATTTTAGATA TGCGATATGA AAATAATAAA | 2640 |
| TTTATAGATA TCTCTGGATA TGGTTCAAAT ATAAGCATTA ATGGAGATGT ATATATTTAT | 2700 |
| TCAACAAATA GAAATCAATT TGGAATATAT AGTAGTAAGC CTAGTGAAGT TAATATAGCT | 2760 |
| CAAAATAATG ATATTATATA CAATGGTAGA TATCAAAATT TTAGTATTAG TTTCTGGGTA | 2820 |
| AGGATTCCTA AATACTTCAA TAAAGTGAAT CTTAATAATG AATATACTAT AATAGATTGT | 2880 |
| ATAAGGAATA ATAATTCAGG ATGGAAAATA TCACTTAATT ATAATAAAAT AATTTGGACT | 2940 |
| TTACAAGATA CTGCTGGAAA TAATCAAAAA CTAGTTTTTA ATTATACACA AATGATTAGT | 3000 |
| ATATCTGATT ATATAAATAA ATGGATTTTT GTAACTATTA CTAATAATAG ATTAGGCAAT | 3060 |
| TCTAGAATTT ACATCAATGG AAATTTAATA GATGAAAAAT CAATTTCGAA TTTAGGTGAT | 3120 |
| ATTCATGTTA GTGATAATAT ATTATTTAAA ATTGTTGGTT GTAATGATAC AAGATATGTT | 3180 |
| GGTATAAGAT ATTTTAAAGT TTTTGATACG GAATTAGGTA AAACAGAAAT TGAGACTTTA | 3240 |
| TATAGTGATG AGCCAGATCC AAGTATCTTA AAAGACTTTT GGGGAAATTA TTTGTTATAT | 3300 |
| AATAAAAGAT ATTATTTATT GAATTTACTA AGAACAGATA AGTCTATTAC TCAGAATTCA | 3360 |
| AACTTTCTAA ATATTAATCA ACAAAGAGGT GTTTATCAGA AACCAAATAT TTTTTCCAAC | 3420 |
| ACTAGATTAT ATACAGGAGT AGAAGTTATT ATAAGAAAAA ATGGATCTAC AGATATATCT | 3480 |
| AATACAGATA ATTTTGTTAG AAAAAATGAT CTGGCATATA TTAATGTAGT AGATCGTGAT | 3540 |
| GTAGAATATC GGCTATATGC TGATATATCA ATTGCAAAAC CAGAGAAAAT AATAAAATTA | 3600 |
| ATAAGAACAT CTAATTCAAA CAATAGCTTA GGTCAAATTA TAGTTATGGA TTCAATAGGA | 3660 |
| AATAATTGCA CAATGAATTT TCAAAACAAT AATGGGGGCA ATATAGGATT ACTAGGTTTT | 3720 |
| CATTCAAATA ATTTGGTTGC TAGTAGTTGG TATTATAACA ATATACGAAA AAATACTAGC | 3780 |
| AGTAATGGAT GCTTTTGGAG TTTTATTTCT AAAGAGCATG GATGGCAAGA AAACTAA. | 3837 |
| BoNT/G, DNA. | |
| SEQ ID NO: 15 | |
| ATGCCAGTTA ATATAAAAAN CTTTAATTAT AATGACCCTA TTAATAATGA TGACATTATT | 60 |
| ATGATGGAAC CATTCAATGA CCCAGGGCCA GGAACATATT ATAAAGCTTT TAGGATTATA | 120 |
| GATCGTATTT GGATAGTACC AGAAAGGTTT ACTTATGGAT TTCAACCTGA CCAATTTAAT | 180 |
| GCCAGTACAG GAGTTTTTAG TAAAGATGTC TACGAATATT ACGATCCAAC TTATTTAAAA | 240 |
| ACCGATGCTG AAAAAGATAA ATTTTTAAAA ACAATGATTA AATTATTTAA TAGAATTAAT | 300 |
| TCAAAACCAT CAGGACAGAG ATTACTGGAT ATGATAGTAG ATGCTATACC TTATCTTGGA | 360 |
| AATGCATCTA CACCGCCCGA CAAATTTGCA GCAAATGTTG CAAATGTATC TATTAATAAA | 420 |
| AAAATTATCC AACCTGGAGC TGAAGATCAA ATAAAAGGTT TAATGACAAA TTTAATAATA | 480 |
| TTTGGACCAG GACCAGTTCT AAGTGATAAT TTTACTGATA GTATGATTAT GAATGGCCAT | 540 |
| TCCCCAATAT CAGAAGGATT TGGTGCAAGA ATGATGATAA GATTTTGTCC TAGTTGTTTA | 600 |
| AATGTATTTA ATAATGTTCA GGAAAATAAA GATACATCTA TATTTAGTAG ACGCGCGTAT | 660 |
| TTTGCAGATC CAGCTCTAAC GTTAATGCAT GAACTTATAC ATGTGTTACA TGGATTATAT | 720 |
| GGAATTAAGA TAAGTAATTT ACCAATTACT CCAAATACAA AAGAATTTTT CATGCAACAT | 780 |
| AGCGATCCTG TACAAGCAGA AGAACTATAT ACATTCGGAG GACATGATCC TAGTGTTATA | 840 |
| AGTCCTTCTA CGGATATGAA TATTTATAAT AAAGCGTTAC AAAATTTTCA AGATATAGCT | 900 |
| AATAGGCTTA ATATTGTTTC AAGTGCCCAA GGGAGTGGAA TTGATATTTC CTTATATAAA | 960 |
| CAAATATATA AAAATAAATA TGATTTTGTT GAAGATCCTA ATGGAAAATA TAGTGTAGAT | 1020 |
| AAGGATAAGT TTGATAAATT ATATAAGGCC TTAATGTTTG GCTTTACTGA AACTAATCTA | 1080 |
| GCTGGTGAAT ATGGAATAAA AACTAGGTAT TCTTATTTTA GTGAATATTT GCCACCGATA | 1140 |
| AAAACTGAAA AATTGTTAGA CAATACAATT TATACTCAAA ATGAAGGCTT TAACATAGCT | 1200 |
| AGTAAAAATC TCAAAACGGA ATTTAATGGT CAGAATAAGG CGGTAAATAA AGAGGCTTAT | 1260 |
| GAAGAAATCA GCCTAGAACA TCTCGTTATA TATAGAATAG CAATGTGCAA GCCTGTAATG | 1320 |
| TACAAAAATA CCGGTAAATC TGAACAGTGT ATTATTGTTA ATAATGAGGA TTTATTTTTC | 1380 |
| ATAGCTAATA AAGATAGTTT TTCAAAAGAT TTAGCTAAAG CAGAAACTAT AGCATATAAT | 1440 |
| ACACAAAATA ATACTATAGA AAATAATTTT TCTATAGATC AGTTGATTTT AGATAATGAT | 1500 |
| TTAAGCAGTG GCATAGACTT ACCAAATGAA AACACAGAAC CATTTACAAA TTTTGACGAC | 1560 |
| ATAGATATCC CTGTGTATAT TAAACAATCT GCTTTAAAAA AAATTTTTGT GGATGGAGAT | 1620 |
| AGCCTTTTTG AATATTTACA TGCTCAAACA TTTCCTTCTA ATATAGAAAA TCTACAACTA | 1680 |
| ACGAATTCAT TAAATGATGC TTTAAGAAAT AATAATAAAG TCTATACTTT TTTTTCTACA | 1740 |
| AACCTTGTTG AAAAAGCTAA TACAGTTGTA GGTGCTTCAC TTTTTGTAAA CTGGGTAAAA | 1800 |
| GGAGTAATAG ATGATTTTAC ATCTGAATCC ACACAAAAAA GTACTATAGA TAAAGTTTCA | 1860 |
| GATGTATCCA TAATTATTCC CTATATAGGA CCTGCTTTGA ATGTAGGAAA TGAAACAGCT | 1920 |
| AAAGAAAATT TTAAAAATGC TTTTGAAATA GGTGGAGCCG CTATCTTAAT GGAGTTTATT | 1980 |
| CCAGAACTTA TTGTACCTAT AGTTGGATTT TTTACATTAG AATCATATGT AGGAAATAAA | 2040 |
| GGGCATATTA TTATGACGAT ATCCAATGCT TTAAAGAAAA GGGATCAAAA ATGGACAGAT | 2100 |
| ATGTATGGTT TGATAGTATC GCAGTGGCTC TCAACGGTTA ATACTCAATT TTATACAATA | 2160 |
| AAAGAAAGAA TGTACAATGC TTTAAATAAT CAATCACAAG CAATAGAAAA AATAATAGAA | 2220 |
| GATCAATATA ATAGATATAG TGAAGAAGAT AAAATGAATA TTAACATTGA TTTTAATGAT | 2280 |
| ATAGATTTTA AACTTAATCA AAGTATAAAT TTAGCAATAA ACAATATAGA TGATTTTATA | 2340 |
| AACCAATGTT CTATATCATA TCTAATGAAT AGAATGATTC CATTAGCTGT AAAAAAGTTA | 2400 |
| AAAGACTTTG ATGATAATCT TAAGAGAGAT TTATTGGAGT ATATAGATAC AAATGAACTA | 2460 |
| TATTTACTTG ATGAAGTAAA TATTCTAAAA TCAAAAGTAA ATAGACACCT AAAAGACAGT | 2520 |
| ATACCATTTG ATCTTTCACT ATATACCAAG GACACAATTT TAATACAAGT TTTTAATAAT | 2580 |
| TATATTAGTA ATATTAGTAG TAATGCTATT TTAAGTTTAA GTTATAGAGG TGGGCGTTTA | 2640 |
| ATAGATTCAT CTGGATATGG TGCAACTATG AATGTAGGTT CAGATGTTAT CTTTAATGAT | 2700 |
| ATAGGAAATG GTCAATTTAA ATTAAATAAT TCTGAAAATA GTAATATTAC GGCACATCAA | 2760 |
| AGTAAATTCG TTGTATATGA TAGTATGTTT GATAATTTTA GCATTAACTT TTGGGTAAGG | 2820 |
| ACTCCTAAAT ATAATAATAA TGATATACAA ACTTATCTTC AAAATGAGTA TACAATAATT | 2880 |
| AGTTGTATAA AAAATGACTC AGGATGGAAA GTATCTATTA AGGGAAATAG AATAATATGG | 2940 |
| ACATTAATAG ATGTTAATGC AAAATCTAAA TCAATATTTT TCGAATATAG TATAAAAGAT | 3000 |
| AATATATCAG ATTATATAAA TAAATGGTTT TCCATAACTA TTACTAATGA TAGATTAGGT | 3060 |
| AACGCAAATA TTTATATAAA TGGAAGTTTG AAAAAAAGTG AAAAAATTTT AAACTTAGAT | 3120 |
| AGAATTAATT CTAGTAATGA TATAGACTTC AAATTAATTA ATTGTACAGA TACTACTAAA | 3180 |
| TTTGTTTGGA TTAAGGATTT TAATATTTTT GGTAGAGAAT TAAATGCTAC AGAAGTATCT | 3240 |
| TCACTATATT GGATTCAATC ATCTACAAAT ACTTTAAAAG ATTTTTGGGG GAATCCTTTA | 3300 |
| AGATACGATA CACAATACTA TCTGTTTAAT CAAGGTATGC AAAATATCTA TATAAAGTAT | 3360 |
| TTTAGTAAAG CTTCTATGGG GGAAACTGCA CCACGTACAA ACTTTAATAA TGCAGCAATA | 3420 |
| AATTATCAAA ATTTATATCT TGGTTTACGA TTTATTATAA AAAAAGCATC AAATTCTCGG | 3480 |
| AATATAAATA ATGATAATAT AGTCAGAGAA GGAGATTATA TATATCTTAA TATTGATAAT | 3540 |
| ATTTCTGATG AATCTTACAG AGTATATGTT TTGGTGAATT CTAAAGAAAT TCAAACTCAA | 3600 |
| TTATTTTTAG CACCCATAAA TGATGATCCT ACGTTCTATG ATGTACTACA AATAAAAAAA | 3660 |
| TATTATGAAA AAACAACATA TAATTGTCAG ATACTTTGCG AAAAAGATAC TAAAACATTT | 3720 |
| GGGCTGTTTG GAATTGGTAA ATTTGTTAAA GATTATGGAT ATGTTTGGGA TACCTATGAT | 3780 |
| AATTATTTTT GCATAAGTCA GTGGTATCTC AGAAGAATAT CTGAAAATAT AAATAAATTA | 3840 |
| AGGTTGGGAT GTAATTGGCA ATTCATTCCC GTGGATGAAG GATGGACAGA ATAA. | 3894 |
| TeNT, DNA. | |
| SEQ ID NO: 16 | |
| ATGCCAATAA CCATAAATAA TTTTAGATAT AGTGATCCTG TTAATAATGA TACAATTATT | 60 |
| ATGATGGAGC CACCATACTG TAAGGGTCTA GATATCTATT ATAAGGCTTT CAAAATAACA | 120 |
| GATCGTATTT GGATAGTGCC GGAAAGGTAT GAATTTGGGA CAAAACCTGA AGATTTTAAC | 180 |
| CCACCATCTT CATTAATAGA AGGTGCATCT GAGTATTACG ATCCAAATTA TTTAAGGACT | 240 |
| GATTCTGATA AAGATAGATT TTTACAAACC ATGGTAAAAC TGTTTAACAG AATTAAAAAC | 300 |
| AATGTAGCAG GTGAAGCCTT ATTAGATAAG ATAATAAATG CCATACCTTA CCTTGGAAAT | 360 |
| TCATATTCCT TACTAGACAA GTTTGATACA AACTCTAATT CAGTATCTTT TAATTTATTA | 420 |
| GAACAAGACC CCAGTGGAGC AACTACAAAA TCAGCAATGC TGACAAATTT AATAATATTT | 480 |
| GGACCTGGGC CTGTTTTAAA TAAAAATGAG GTTAGAGGTA TTGTATTGAG GGTAGATAAT | 540 |
| AAAAATTACT TCCCATGTAG AGATGGTTTT GGCTCAATAA TGCAAATGGC ATTTTGCCCA | 600 |
| GAATATGTAC CTACCTTTGA TAATGTAATA GAAAATATTA CGTCACTCAC TATTGGCAAA | 660 |
| AGCAAATATT TTCAAGATCC AGCATTACTA TTAATGCACG AACTTATACA TGTACTACAT | 720 |
| GGTTTATACG GAATGCAGGT ATCAAGCCAT GAAATTATTC CATCCAAACA AGAAATTTAT | 780 |
| ATGCAGCATA CATATCCAAT AAGTGCTGAA GAACTATTCA CTTTTGGCGG ACAGGATGCT | 840 |
| AATCTTATAA GTATTGATAT AAAAAACGAT TTATATGAAA AAACTTTAAA TGATTATAAA | 900 |
| GCTATAGCTA ACAAACTTAG TCAAGTCACT AGCTGCAATG ATCCCAACAT TGATATTGAT | 960 |
| AGCTACAAAC AAATATATCA ACAAAAATAT CAATTCGATA AAGATAGCAA TGGACAATAT | 1020 |
| ATTGTAAATG AGGATAAATT TCAGATACTA TATAATAGCA TAATGTATGG TTTTACAGAG | 1080 |
| ATTGAATTGG GAAAAAAATT TAATATAAAA ACTAGACTTT CTTATTTTAG TATGAATCAT | 1140 |
| GACCCTGTAA AAATTCCAAA TTTATTAGAT GATACAATTT ACAATGATAC AGAAGGATTT | 1200 |
| AATATAGAAA GCAAAGATCT GAAATCTGAA TATAAAGGAC AAAATATGAG GGTAAATACA | 1260 |
| AATGCTTTTA GAAATGTTGA TGGATCAGGC CTAGTTTCAA AACTTATTGG CTTATGTAAA | 1320 |
| AAAATTATAC CACCAACAAA TATAAGAGAA AATTTATATA ATAGAACTGC ATCATTAACA | 1380 |
| GATTTAGGAG GAGAATTATG TATAAAAATT AAAAATGAAG ATTTAACTTT TATAGCTGAA | 1440 |
| AAAAATAGCT TTTCAGAAGA ACCATTTCAA GATGAAATAG TTAGTTATAA TACAAAAAAT | 1500 |
| AAACCATTAA ATTTTAATTA TTCGCTAGAT AAAATTATTG TAGATTATAA TCTACAAAGT | 1560 |
| AAAATTACAT TACCTAATGA TAGGACAACC CCAGTTACAA AAGGAATTCC ATATGCTCCA | 1620 |
| GAATATAAAA GTAATGCTGC AAGTACAATA GAAATACATA ATATTGATGA CAATACAATA | 1680 |
| TATCAATATT TGTATGCTCA AAAATCTCCT ACAACTCTAC AAAGAATAAC TATGACTAAT | 1740 |
| TCTGTTGATG ACGCATTAAT AAATTCCACC AAAATATATT CATATTTTCC ATCTGTAATC | 1800 |
| AGTAAAGTTA ACCAAGGTGC ACAAGGAATT TTATTCTTAC AGTGGGTGAG AGATATAATT | 1860 |
| GATGATTTTA CCAATGAATC TTCACAAAAA ACTACTATTG ATAAAATTTC AGATGTATCC | 1920 |
| ACTATTGTTC CTTATATAGG ACCCGCATTA AACATTGTAA AACAAGGCTA TGAGGGAAAC | 1980 |
| TTTATAGGCG CTTTAGAAAC TACCGGAGTG GTTTTATTAT TAGAATATAT TCCAGAAATT | 2040 |
| ACTTTACCAG TAATTGCAGC TTTATCTATA GCAGAAAGTA GCACACAAAA AGAAAAGATA | 2100 |
| ATAAAAACAA TAGATAACTT TTTAGAAAAA AGATATGAAA AATGGATTGA AGTATATAAA | 2160 |
| CTAGTAAAAG CAAAATGGTT AGGCACAGTT AATACGCAAT TCCAAAAAAG AAGTTATCAA | 2220 |
| ATGTATAGAT CTTTAGAATA TCAAGTAGAT GCAATAAAAA AAATAATAGA CTATGAATAT | 2280 |
| AAAATATATT CAGGACCTGA TAAGGAACAA ATTGCCGACG AAATTAATAA TCTGAAAAAC | 2340 |
| AAACTTGAAG AAAAGGCTAA TAAAGCAATG ATAAACATAA ATATATTTAT GAGGGAAAGT | 2400 |
| TCTAGATCAT TTTTAGTTAA TCAAATGATT AACGAAGCTA AAAAGCAGTT ATTAGAGTTT | 2460 |
| GATACTCAAA GCAAAAATAT TTTAATGCAG TATATAAAAG CAAATTCTAA ATTTATAGGT | 2520 |
| ATAACTGAAC TAAAAAAATT AGAATCAAAA ATAAACAAAG TTTTTTCAAC ACCAATTCCA | 2580 |
| TTTTCTTATT CTAAAAATCT GGATTGTTGG GTTGATAATG AAGAAGATAT AGATGTTATA | 2640 |
| TTAAAAAAGA GTACAATTTT AAATTTAGAT ATTAATAATG ATATTATATC AGATATATCT | 2700 |
| GGGTTTAATT CATCTGTAAT AACATATCCA GATGCTCAAT TGGTGCCCGG AATAAATGGC | 2760 |
| AAAGCAATAC ATTTAGTAAA CAATGAATCT TCTGAAGTTA TAGTGCATAA AGCTATGGAT | 2820 |
| ATTGAATATA ATGATATGTT TAATAATTTT ACCGTTAGCT TTTGGTTGAG GGTTCCTAAA | 2880 |
| GTATCTGCTA GTCATTTAGA ACAATATGGC ACAAATGAGT ATTCAATAAT TAGCTCTATG | 2940 |
| AAAAAACATA GTCTATCAAT AGGATCTGGT TGGAGTGTAT CACTTAAAGG TAATAACTTA | 3000 |
| ATATGGACTT TAAAAGATTC CGCGGGAGAA GTTAGACAAA TAACTTTTAG GGATTTACCT | 3060 |
| GATAAATTTA ATGCTTATTT AGCAAATAAA TGGGTTTTTA TAACTATTAC TAATGATAGA | 3120 |
| TTATCTTCTG CTAATTTGTA TATAAATGGA GTACTTATGG GAAGTGCAGA AATTACTGGT | 3180 |
| TTAGGAGCTA TTAGAGAGGA TAATAATATA ACATTAAAAC TAGATAGATG TAATAATAAT | 3240 |
| AATCAATACG TTTCTATTGA TAAATTTAGG ATATTTTGCA AAGCATTAAA TCCAAAAGAG | 3300 |
| ATTGAAAAAT TATACACAAG TTATTTATCT ATAACCTTTT TAAGAGACTT CTGGGGAAAC | 3360 |
| CCTTTACGAT ATGATACAGA ATATTATTTA ATACCAGTAG CTTCTAGTTC TAAAGATGTT | 3420 |
| CAATTGAAAA ATATAACAGA TTATATGTAT TTGACAAATG CGCCATCGTA TACTAACGGA | 3480 |
| AAATTGAATA TATATTATAG AAGGTTATAT AATGGACTAA AATTTATTAT AAAAAGATAT | 3540 |
| ACACCTAATA ATGAAATAGA TTCTTTTGTT AAATCAGGTG ATTTTATTAA ATTATATGTA | 3600 |
| TCATATAACA ATAATGAGCA CATTGTAGGT TATCCGAAAG ATGGAAATGC CTTTAATAAT | 3660 |
| CTTGATAGAA TTCTAAGAGT AGGTTATAAT GCCCCAGGTA TCCCTCTTTA TAAAAAAATG | 3720 |
| GAAGCAGTAA AATTGCGTGA TTTAAAAACC TATTCTGTAC AACTTAAATT ATATGATGAT | 3780 |
| AAAAATGCAT CTTTAGGACT AGTAGGTACC CATAATGGTC AAATAGGCAA CGATCCAAAT | 3840 |
| AGGGATATAT TAATTGCAAG CAACTGGTAC TTTAATCATT TAAAAGATAA AATTTTAGGA | 3900 |
| TGTGATTGGT ACTTTGTACC TACAGATGAA GGATGGACAA ATGATTAA. | 3948 |

DNA, BoNT/A1(0) LC
SEQ ID NO: 17
ATGCCATTCGTCAACAAGCAATTCAACTACAAAGACCCAGTCAACGGCGTC
GACATCGCATACATCAAGATTCCGAACGCCGGTCAAATGCAGCCGGTTAAG
GCTTTTAAGATCCACAACAAGATTTGGGTTATCCCGGAGCGTGACACCTTCA
CGAACCCGGAAGAAGGCGATCTGAACCCGCCACCGGAAGCGAAGCAAGTC
CCTGTCAGCTACTACGATTCGACGTACCTGAGCACGGATAACGAAAAAGAT
AACTACCTGAAAGGTGTGACCAAGCTGTTCGAACGTATCTACAGCACGGAT
CTGGGTCGCATGCTGCTGACTAGCATTGTTCGCGGTATCCCGTTCTGGGGTG
GTAGCACGATTGACACCGAACTGAAGGTTATCGACACTAACTGCATTAACG
TTATTCAACCGGATGGTAGCTATCGTAGCGAAGAGCTGAATCTGGTCATCAT
TGGCCCGAGCGCAGACATTATCCAATTCGAGTGCAAGAGCTTTGGTCACGA
GGTTCTGAATCTGACCCGCAATGGCTATGGTAGCACCCAGTACATTCGTTTT
TCGCCGGATTTTACCTTCGGCTTTGAAGAGAGCCTGGAGGTTGATACCAATC
CGTTGCTGGGTGCGGGCAAATTCGCTACCGATCCGGCTGTCACGCTGGCCCA
TcAACTGATCtACGCAGGCCACCGCCTGTACGGCATTGCCATCAACCCAAAC
CGTGTGTTCAAGGTTAATACGAATGCATACTACGAGATGAGCGGCCTGGAA
GTCAGCTTCGAAGAACTGCGCACCTTCGGTGGCCATGACGCTAAATTCATTG
ACAGCTTGCAAGAGAATGAGTTCCGTCTGTACTACTATAACAAATTCAAAG
ACATTGCAAGCACGTTGAACAAGGCCAAAAGCATCGTTGGTACTACCGCGT
CGTTGCAGTATATGAAGAATGTGTTTAAAGAGAAGTACCTGCTGTCCGAGG
ATACCTCCGGCAAGTTTAGCGTTGATAAGCTGAAGTTTGACAAACTGTACA
AGATGCTGACCGAGATTTACACCGAGGACAACTTTGTGAAATTCTTCAAAG
TGTTGAATCGTAAAACCTATCTGAATTTTGACAAAGCGGTTTTCAAGATTAA
CATCGTGCCGAAGGTGAACTACACCATCTATGACGGTTTTAACCTGCGTAAC
ACCAACCTGGCGGCGAACTTTAACGGTCAGAATACGGAAATCAACAACATG
AATTTCACGAAGTTGAAGAACTTCACGGGTCTGTTCGAGTTCTATAAGCTGC
TGTGCGTGCGCGGTATCATCACCAGCAAA,
DNA, BoNT/A1(0) Activation loop
SEQ ID NO: 18
ACCAAAAGCCTGGACAAAGGCTACAACAAG,
DNA, BoNT/A1(0) HC
SEQ ID NO: 19
GCGCTGAATGACCTGTGCATTAAGGTAAACAATTGGGATCTGTTCTTTTCGC
CATCCGAAGATAATTTTACCAACGACCTGAACAAGGGTGAAGAAATCACCA
GCGATACGAATATTGAAGCAGCGGAAGAGAATATCAGCCTGGATCTGATCC
AGCAGTACTATCTGACCTTTAACTTCGACAATGAACCGGAGAACATTAGCA
TTGAGAATCTGAGCAGCGACATTATCGGTCAGCTGGAACTGATGCCGAATA
TCGAACGTTTCCCGAACGGCAAAAAGTACGAGCTGGACAAGTACACTATGT
TCCATTACCTGCGTGCACAGGAGTTTGAACACGGTAAAAGCCGTATCGCGC
TGACCAACAGCGTTAACGAGGCCCTGCTGAACCCGAGCCGTGTCTATACCTT
CTTCAGCAGCGACTATGTTAAGAAAGTGAACAAAGCCACTGAGGCCGCGAT
GTTCCTGGGCTGGGTGGAACAGCTGGTATATGACTTCACGGACGAGACGAG
CGAAGTGAGCACTACCGACAAAATTGCTGATATTACCATCATTATCCCGTAT
ATTGGTCCGGCACTGAACATTGGCAACATGCTGTACAAAGACGATTTTGTG
GGTGCCCTGATCTTCTCCGGTGCCGTGATTCTGCTGGAGTTCATTCCGGAGA
TTGCGATCCCGGTGTTGGGTACCTTCGCGCTGGTGTCCTACATCGCGAATAA
GGTTCTGACGGTTCAGACCATCGATAACGCGCTGTCGAAACGTAATGAAAA
ATGGGACGAGGTTTACAAATACATTGTTACGAATTGGCTGGCGAAAGTCAA
TACCCAGATCGACCTGATCCGTAAGAAAATGAAAGAGGCGCTGGAGAATCA
GGCGGAGGCCACCAAAGCAATTATCAACTACCAATACAACCAGTACACGGA
AGAAGAGAAGAATAACATTAACTTCAATATCGATGATTTGAGCAGCAAGCT
GAATGAATCTATCAACAAAGCGATGATCAATATCAACAAGTTTTTGAATCA
GTGTAGCGTTTCGTACCTGATGAATAGCATGATTCCGTATGGCGTCAAACGT
CTGGAGGACTTCGACGCCAGCCTGAAAGATGCGTTGCTGAAATACATTTAC
GACAATCGTGGTACGCTGATTGGCCAAGTTGACCGCTTGAAAGACAAAGTT
AACAATACCCTGAGCACCGACATCCCATTTCAACTGAGCAAGTATGTTGAT
AATCAACGTCTGTTGAGCACTTTCACCGAGTATATCAAAAACATCATCAATA
CTAGCATTCTGAACCTGCGTTACGAGAGCAATCATCTGATTGATCTGAGCCG
TTATGCAAGCAAGATCAACATCGGTAGCAAGGTCAATTTTGACCCGATCGA
TAAGAACCAGATCCAGCTGTTTAATCTGGAATCGAGCAAAATTGAGGTTAT
CCTGAAAAACGCCATTGTCTACAACTCCATGTACGAGAATTTCTCCACCAGC
TTCTGGATTCGCATCCCGAAATACTTCAACAGCATTAGCCTGAACAACGAGT
ATACTATCATCAACTGTATGGAGAACAACAGCGGTTGGAAGGTGTCTCTGA
ACTATGGTGAGATCATTTGGACCTTGCAGGACACCCAAGAGATCAAGCAGC
GCGTCGTGTTCAAGTACTCTCAAATGATCAACATTTCCGATTACATTAATCG
TTGGATCTTCGTGACCATTACGAATAACCGTCTGAATAACAGCAAGATTTAC
ATCAATGGTCGCTTGATCGATCAGAAACCGATTAGCAACCTGGGTAATATC
CACGCAAGCAACAACATTATGTTCAAATTGGACGGTTGCCGCGATACCCAT
CGTTATATCTGGATCAAGTATTTCAACCTGTTTGATAAAGAACTGAATGAGA
AGGAGATCAAAGATTTGTATGACAACCAATCTAACAGCGGCATTTTGAAGG
ACTTCTGGGGCGATTATCTGCAATACGATAAGCCGTACTATATGCTGAACCT
GTATGATCCGAACAAATATGTGGATGTCAATAATGTGGGTATTCGTGGTTAC
ATGTATTTGAAGGGTCCGCGTGGCAGCGTTATGACGACCAACATTTACCTGA
ACTCTAGCCTGTACCGTGGTACGAAATTCATCATTAAGAAATATGCCAGCG
GCAACAAAGATAACATTGTGCGTAATAACGATCGTGTCTACATCAACGTGG
TCGTGAAGAATAAAGAGTACCGTCTGGCGACCAACGCTTCGCAGGCGGGTG
TTGAGAAAATTCTGAGCGCGTTGGAGATCCCTGATGTCGGTAATCTGAGCC
AAGTCGTGGTTATGAAGAGCAAGAACGACCAGGGTATCACTAACAAGTGCA
AGATGAACCTGCAAGACAACAATGGTAACGACATCGGCTTTATTGGTTTCC
ACCAGTTCAACAATATTGCTAAACTGGTAGCGAGCAATTGGTACAATCGTC
AGATTGAGCGCAGCAGCCGTACTTTGGGCTGTAGCTGGGAGTTTATCCCGGT
CGATGATGGTTGGGGCGAACGTCCGCTGTAA,
DNA, primer
SEQ ID NO: 20
ATACACCATGGTATGCCATTCGTCAACAAGCAATT,
DNA, primer
SEQ ID NO: 21
GCTTTTGGATCCGGTTTATTTGCTGGTGATGATACCGCGC,
DNA, primer
SEQ ID NO: 22
ACAAGCATATGGCGCTGAATGACCTGTGCATTAAG,
DNA, primer
SEQ ID NO: 23
AAGCTTCTCGAGTCATTACAGCGGACGTTCGCCCC,

1. A method for producing a di-chain clostridial neurotoxin, comprising separately expressing in a heterologous host cell a first gene encoding a clostridial neurotoxin light chain and a second gene encoding a clostridial neurotoxin heavy chain, wherein said first and second genes are expressed in an oxidizing environment of said host cell.

2. A method according to claim 1, wherein said host cell is a prokaryote cell.

3. A method according to claim 2, wherein said oxidizing environment is the cytoplasm of said host prokaryote cell.

4. A method according to claim 2 or 3, wherein said prokaryote cell is a prokaryote cell in which at least one gene involved in disulphide bond formation is overexpressed by in the cytoplasm as compared to an otherwise identical wild-type cell and/or at least one gene involved in disulphide bond reduction is repressed as compared to an otherwise identical wild-type cell.

5. A method according to claim 4, wherein said prokaryote cell is an E. coli cell from a strain selected from AD494, BL21trxB, Origami, Rosetta-gami and Shuffle strains.

6. A method according to any one of claims 1 to 5, wherein said clostridial neurotoxin light chain is selected from a BoNT type A, type B, type C, type D, type E, type F or type G or a TeNT light chain, and wherein said clostridial neurotoxin heavy chain is selected from a BoNT type A, type B, type C, type D, type E, type F or type G or a TeNT heavy chain.

7. A method according to any one of claims 1 to 6, wherein said clostridial neurotoxin light chain and said clostridial neurotoxin heavy chain are from the same BoNT serotype or subtype.

8. A method according to any one of claims 1 to 6, wherein said clostridial neurotoxin light chain and said clostridial neurotoxin heavy chain are from a different BoNT serotype or subtype.

9. A method according to any one of claims 1 to 8, wherein said first gene encoding a clostridial neurotoxin light chain and said second gene encoding a clostridial neurotoxin heavy chain are present on the same vector.

10. A method according to any one of claims 1 to 8, wherein said first gene encoding a clostridial neurotoxin light chain and said second gene encoding a clostridial neurotoxin heavy chain are present on different vectors.

11. A method according to any one of claims 1 to 10 further comprising a step of recovering said di-chain clostridial neurotoxin from said host cell.

12. Cell comprising a first gene encoding a clostridial neurotoxin light chain, and a second gene encoding a clostridial neurotoxin heavy chain, wherein, wherein said first and second genes are expressed in an oxidizing environment of said cell.

13. Kit comprising
a. a cell comprising an oxidizing environment,
b. a first gene encoding a clostridial neurotoxin light chain, and
c. a second gene encoding a clostridial neurotoxin heavy chain,
wherein said first and second genes are suitable for separately expressing a clostridial neurotoxin light and a heavy chain in said oxidizing environment of said cell.

14. Di-chain clostridial neurotoxin obtained by the method according to any one of claims 1 to 11.

15. Pharmaceutical composition comprising a di-chain clostridial neurotoxin according to claim 14.

16. Use of a host cell which has an oxidative cytoplasm for producing a di-chain clostridial neurotoxin, wherein said host cell comprises a first gene encoding a clostridial neurotoxin light chain and a second gene encoding a clostridial neurotoxin heavy chain, wherein said first and second genes are expressed in the oxidative cytoplasm of said host cell.
